SEQUENCE LISTING (70 sequences)

SEQ ID NO: 1 - protein, 213 aa, synthetic construct
  REGION 1..213: note = Artificial construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 2 - protein, 240 aa, synthetic construct
  REGION 1..240: note = Artificial construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 3 - protein, 121 aa, synthetic construct
  REGION 1..121: note = Artificial construct
QVQLQQSGPELVKPGASVKMSCKASGYRFSDYVISWVKQRSGQGLEWIGEIYPGDGNTYYNEMFKGRATLTADKSPNTAYMKLSSLTSEDSALYFCARGSYTYDNYAMDFWGQGTSVTVSS

SEQ ID NO: 4 - protein, 112 aa, synthetic construct
  REGION 1..112: note = Artificial construct
DVLMTQTPLSLPVSLGDQASISCRSSQNIVHSNGNTYLEWYLQKPGQSPKLLIYKVSNRFSGVPDRFSGSGSGTDFTLKISRVEAEDLGLYYCFQGSHIPWTFGGGTKLEIK

SEQ ID NO: 5 - protein, 119 aa, synthetic construct
  REGION 1..119: note = Artificial construct
EVQLQQPGSELVRPGASVKLSCKASGYTFTSYWMHWMKQRPGQGLEWIGHIYLGGGATNYAEKFRSKATLTADTSSSTAYMQLSSLTSEDSAVYYCTRSQPYYYGMDSWGQGTSVTVSS

SEQ ID NO: 6 - protein, 107 aa, synthetic construct
  REGION 1..107: note = Artificial construct
DIQMTQTPSSLSASLGDRVTISCRASQDISNYLNWYQQKPDGTVKLLIYYKSRLHSGVPSRFSGSGSGTEYSLTINNLEQEDFATYFCHQGHTIPFTFGSGTKLEIK

SEQ ID NO: 7 - protein, 668 aa, synthetic construct
  REGION 1..668: note = Artificial construct
DVLMTQTPLSLPVSLGDQASISCRSSQNIVHSNGNTYLEWYLQKPGQSPKLLIYKVSNRFSGVPDRFSGSGSGTDFTLKISRVEAEDLGLYYCFQGSHIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQPGSELVRPGASVKLSCKASGYTFTSYWMHWMKQRPGQGLEWIGHIYLGGGATNYAEKFRSKATLTADTSSSTAYMQLSSLTSEDSAVYYCTRSQPYYYGMDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 224 aa, synthetic construct
  REGION 1..224: note = Artificial construct
QVQLQQSGPELVKPGASVKMSCKASGYRFSDYVISWVKQRSGQGLEWIGEIYPGDGNTYYNEMFKGRATLTADKSPNTAYMKLSSLTSEDSALYFCARGSYTYDNYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 214 aa, synthetic construct
  REGION 1..214: note = Artificial construct
DIQMTQTPSSLSASLGDRVTISCRASQDISNYLNWYQQKPDGTVKLLIYYKSRLHSGVPSRFSGSGSGTEYSLTINNLEQEDFATYFCHQGHTIPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 449 aa, synthetic construct
  REGION 1..449: note = Artificial construct
EVQLQQPGSELVRPGASVKLSCKASGYTFTSYWMHWMKQRPGQGLEWIGHIYLGGGATNYAEKFRSKATLTADTSSSTAYMQLSSLTSEDSAVYYCTRSQPYYYGMDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 11 - protein, 214 aa, synthetic construct; identical to SEQ ID NO: 9
  REGION 1..214: note = Artificial construct

SEQ ID NO: 12 - protein, 451 aa, synthetic construct
  REGION 1..451: note = Artificial construct
QVQLQQSGPELVKPGASVKMSCKASGYRFSDYVISWVKQRSGQGLEWIGEIYPGDGNTYYNEMFKGRATLTADKSPNTAYMKLSSLTSEDSALYFCARGSYTYDNYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 13 - protein, 219 aa, synthetic construct
  REGION 1..219: note = Artificial construct
DVLMTQTPLSLPVSLGDQASISCRSSQNIVHSNGNTYLEWYLQKPGQSPKLLIYKVSNRFSGVPDRFSGSGSGTDFTLKISRVEAEDLGLYYCFQGSHIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 119 aa, synthetic construct
  REGION 1..119: note = Artificial construct
EVQLVQSGAEVKKPGASVKVSCKASGYTFTSYWMHWVRQAPGQGLEWMGHIYLGGGATNYAEKFRSRVTMTRDTSTSTVYMELSSLRSEDTAVYYCARSQPYYYGMDSWGQGTTVTVSS

SEQ ID NO: 15 - protein, 119 aa, synthetic construct
  REGION 1..119: note = Artificial construct
EVQLVQSGAEVKKPGASVKVSCKASGYTFTSYWMHWVRQAPGQGLEWMGHIYLGGGATNYAEKFRSRVTLTADTSTSTVYMELSSLRSEDTAVYYCARSQPYYYGMDSWGQGTTVTVSS

SEQ ID NO: 16 - protein, 119 aa, synthetic construct
  REGION 1..119: note = Artificial construct
EVQLVQSGAEVKKPGASVKVSCKASGYTFTSYWMHWVRQAPGQGLEWMGHIYLGGGATNYAEKFRSKATLTADTSTSTVYMELSSLRSEDTAVYYCARSQPYYYGMDSWGQGTTVTVSS

SEQ ID NO: 17 - protein, 119 aa, synthetic construct
  REGION 1..119: note = Artificial construct
EVQLVQSGAEVKKPGASVKVSCKASGYTFTSYWMHWVKQAPGQGLEWIGHIYLGGGATNYAEKFRSKATLTADTSTSTVYMELSSLRSEDTAVYYCARSQPYYYGMDSWGQGTTVTVSS

SEQ ID NO: 18 - protein, 119 aa, synthetic construct
  REGION 1..119: note = Artificial construct
EVQLVQSGAEVKKPGASVKVSCKASGYTFTSYWMHWMKQAPGQGLEWIGHIYLGGGATNYAEKFRSKATLTADTSTSTAYMELSSLRSEDTAVYYCARSQPYYYGMDSWGQGTTVTVSS

SEQ ID NO: 19 - protein, 107 aa, synthetic construct
  REGION 1..107: note = Artificial construct
DIQMTQSPSSLSASVGDRVTITCRASQDISNYLNWYQQKPGKAPKLLIYYKSRLHSGVPSRFSGSGSGTDFTLTISSLQPEDFATYYCHQGHTIPFTFGQGTKLEIK

SEQ ID NO: 20 - protein, 107 aa, synthetic construct
  REGION 1..107: note = Artificial construct
DIQMTQSPSSLSASVGDRVTITCRASQDISNYLNWYQQKPGKAPKLLIYYKSRLHSGVPSRFSGSGSGTEYTLTISSLQPEDFATYYCHQGHTIPFTFGQGTKLEIK

SEQ ID NO: 21 - protein, 121 aa, synthetic construct
  REGION 1..121: note = Artificial construct
EVQLVQSGAEVKKPGASVKVSCKASGYTFTDYVISWVRQATGQGLEWMGEIYPGDAQTYYNEMFKGRVTMTRDTSISTAYMELSSLRSEDTAVYYCARGSYTYDNYAMDFWGQGTTVTVSS

SEQ ID NO: 22 - protein, 121 aa, synthetic construct
  REGION 1..121: note = Artificial construct
EVQLVQSGAEVKKPGASVKVSCKASGYTFTDYVISWVRQATGQGLEWMGEIYPGDAQTYYNEMFKGRVTMTADTSISTAYMELSSLRSEDTAVYYCARGSYTYDNYAMDFWGQGTTVTVSS

SEQ ID NO: 23 - protein, 121 aa, synthetic construct
  REGION 1..121: note = Artificial construct
EVQLVQSGAEVKKPGASVKVSCKASGYRFSDYVISWVRQATGQGLEWMGEIYPGDAQTYYNEMFKGRVTMTADTSISTAYMELSSLRSEDTAVYYCARGSYTYDNYAMDFWGQGTTVTVSS

SEQ ID NO: 24 - protein, 121 aa, synthetic construct
  REGION 1..121: note = Artificial construct
EVQLVQSGAEVKKPGASVKVSCKASGYTFTDYVISWVRQATGQGLEWMGEIYPGDAQTYYNEMFKGRVTLTADTSISTAYMELSSLRSEDTAVYYCARGSYTYDNYAMDFWGQGTTVTVSS

SEQ ID NO: 25 - protein, 121 aa, synthetic construct
  REGION 1..121: note = Artificial construct
EVQLVQSGAEVKKPGASVKVSCKASGYTFTDYVISWVKQATGQGLEWIGEIYPGDAQTYYNEMFKGRATLTADKSISTAYMELSSLRSEDTAVYYCARGSYTYDNYAMDFWGQGTTVTVSS

SEQ ID NO: 26 - protein, 121 aa, synthetic construct
  REGION 1..121: note = Artificial construct
EVQLVQSGAEVKKPGASVKVSCKASGYRFSDYVISWVKQATGQGLEWIGEIYPGDAQTYYNEMFKGRATLTADKSISTAYMELSSLRSEDTAVYFCARGSYTYDNYAMDFWGQGTTVTVSS

SEQ ID NO: 27 - protein, 121 aa, synthetic construct
  REGION 1..121: note = Artificial construct
EVQLVQSGAEVKKPGASVKVSCKASGYTFTDYVISWVRQATGQGLEWMGEIYPGDGNTYYNEMFKGRVTMTRDTSISTAYMELSSLRSEDTAVYYCARGSYTYDNYAMDFWGQGTTVTVSS

SEQ ID NO: 28 - protein, 121 aa, synthetic construct
  REGION 1..121: note = Artificial construct
EVQLVQSGAEVKKPGASVKVSCKASGYTFTDYVISWVRQATGQGLEWMGEIYPGDANTYYNEMFKGRVTMTRDTSISTAYMELSSLRSEDTAVYYCARGSYTYDNYAMDFWGQGTTVTVSS

SEQ ID NO: 29 - protein, 121 aa, synthetic construct
  REGION 1..121: note = Artificial construct
EVQLVQSGAEVKKPGASVKVSCKASGYTFTDYVISWVRQATGQGLEWMGEIYPGEGNTYYNEMFKGRVTMTRDTSISTAYMELSSLRSEDTAVYYCARGSYTYDNYAMDFWGQGTTVTVSS

SEQ ID NO: 30 - protein, 112 aa, synthetic construct
  REGION 1..112: note = Artificial construct
DVVMTQSPLSLPVTLGQPASISCRSSQNIVHSEGQTYLEWFQQRPGQSPRRLIYKVSNRFSGVPDRFSGSGSGTDFTLKISRVEAEDVGVYYCFQGSHIPWTFGGGTKVEIK

SEQ ID NO: 31 - protein, 112 aa, synthetic construct
  REGION 1..112: note = Artificial construct
DVVMTQSPLSLPVTLGQPASISCRSSQNIVHSEGQTYLEWFQQRPGQSPRLLIYKVSNRFSGVPDRFSGSGSGTDFTLKISRVEAEDVGVYYCFQGSHIPWTFGGGTKVEIK

SEQ ID NO: 32 - protein, 112 aa, synthetic construct
  REGION 1..112: note = Artificial construct
DVVMTQTPLSLPVTLGQPASISCRSSQNIVHSEGQTYLEWYQQRPGQSPRLLIYKVSNRFSGVPDRFSGSGSGTDFTLKISRVEAEDVGVYYCFQGSHIPWTFGGGTKVEIK

SEQ ID NO: 33 - protein, 112 aa, synthetic construct
  REGION 1..112: note = Artificial construct
DVVMTQSPLSLPVTLGQPASISCRSSQNIVHSEGQTYLEWYQQRPGQSPRLLIYKVSNRFSGVPDRFSGSGSGTDFTLKISRVEAEDVGVYYCFQGSHIPWTFGGGTKVEIK

SEQ ID NO: 34 - protein, 112 aa, synthetic construct
  REGION 1..112: note = Artificial construct
DVVMTQTPLSLPVTLGQPASISCRSSQNIVHSEGQTYLEWFQQRPGQSPRLLIYKVSNRFSGVPDRFSGSGSGTDFTLKISRVEAEDVGVYYCFQGSHIPWTFGGGTKVEIK

SEQ ID NO: 35 - protein, 112 aa, synthetic construct
  REGION 1..112: note = Artificial construct
DVVMTQSPLSLPVTLGQPASISCRSSQNIVHSEGQTYLEWYLQRPGQSPRLLIYKVSNRFSGVPDRFSGSGSGTDFTLKISRVEAEDVGVYYCFQGSHIPWTFGGGTKVEIK

SEQ ID NO: 36 - protein, 112 aa, synthetic construct
  REGION 1..112: note = Artificial construct
DVVMTQSPLSLPVTLGQPASISCRSSQNIVHSNGNTYLEWFQQRPGQSPRLLIYKVSNRFSGVPDRFSGSGSGTDFTLKISRVEAEDVGVYYCFQGSHIPWTFGGGTKVEIK

SEQ ID NO: 37 - protein, 112 aa, synthetic construct
  REGION 1..112: note = Artificial construct
DVVMTQTPLSLPVTLGQPASISCRSSQNIVHSNGNTYLEWYQQRPGQSPRLLIYKVSNRFSGVPDRFSGSGSGTDFTLKISRVEAEDVGVYYCFQGSHIPWTFGGGTKVEIK

SEQ ID NO: 38 - protein, 112 aa, synthetic construct
  REGION 1..112: note = Artificial construct
DVVMTQSPLSLPVTLGQPASISCRSSQNIVHSNGNTYLEWYQQRPGQSPRLLIYKVSNRFSGVPDRFSGSGSGTDFTLKISRVEAEDVGVYYCFQGSHIPWTFGGGTKVEIK

SEQ ID NO: 39 - protein, 112 aa, synthetic construct
  REGION 1..112: note = Artificial construct
DVVMTQTPLSLPVTLGQPASISCRSSQNIVHSNGNTYLEWFQQRPGQSPRLLIYKVSNRFSGVPDRFSGSGSGTDFTLKISRVEAEDVGVYYCFQGSHIPWTFGGGTKVEIK

SEQ ID NO: 40 - protein, 112 aa, synthetic construct
  REGION 1..112: note = Artificial construct
DVVMTQSPLSLPVTLGQPASISCRSSQNIVHSNGNTYLEWYLQRPGQSPRLLIYKVSNRFSGVPDRFSGSGSGTDFTLKISRVEAEDVGVYYCFQGSHIPWTFGGGTKVEIK

SEQ ID NO: 41 - protein, 112 aa, synthetic construct
  REGION 1..112: note = Artificial construct
DVVMTQSPLSLPVTLGQPASISCRSSQNIVHSNANTYLEWFQQRPGQSPRLLIYKVSNRFSGVPDRFSGSGSGTDFTLKISRVEAEDVGVYYCFQGSHIPWTFGGGTKVEIK

SEQ ID NO: 42 - protein, 112 aa, synthetic construct
  REGION 1..112: note = Artificial construct
DVVMTQSPLSLPVTLGQPASISCRSSQNIVHSNGQTYLEWFQQRPGQSPRLLIYKVSNRFSGVPDRFSGSGSGTDFTLKISRVEAEDVGVYYCFQGSHIPWTFGGGTKVEIK

SEQ ID NO: 43 - protein, 112 aa, synthetic construct
  REGION 1..112: note = Artificial construct
DVVMTQSPLSLPVTLGQPASISCRSSQNIVHSNAQTYLEWFQQRPGQSPRLLIYKVSNRFSGVPDRFSGSGSGTDFTLKISRVEAEDVGVYYCFQGSHIPWTFGGGTKVEIK

SEQ ID NO: 44 - protein, 5 aa, synthetic construct
  REGION 1..5: note = Artificial construct
DYVIS

SEQ ID NO: 45 - protein, 17 aa, synthetic construct
  REGION 1..17: note = Artificial construct
EIYPGDGNTYYNEMFKG

SEQ ID NO: 46 - protein, 12 aa, synthetic construct
  REGION 1..12: note = Artificial construct
GSYTYDNYAMDF

SEQ ID NO: 47 - protein, 17 aa, synthetic construct
  REGION 1..17: note = Artificial construct
EIYPGDAQTYYNEMFKG

SEQ ID NO: 48 - protein, 17 aa, synthetic construct
  REGION 1..17: note = Artificial construct
EIYPGDANTYYNEMFKG

SEQ ID NO: 49 - protein, 17 aa, synthetic construct
  REGION 1..17: note = Artificial construct
EIYPGEGNTYYNEMFKG

SEQ ID NO: 50 - protein, 16 aa, synthetic construct
  REGION 1..16: note = Artificial construct
RSSQNIVHSNGNTYLE

SEQ ID NO: 51 - protein, 7 aa, synthetic construct
  REGION 1..7: note = Artificial construct
KVSNRFS

SEQ ID NO: 52 - protein, 9 aa, synthetic construct
  REGION 1..9: note = Artificial construct
FQGSHIPWT

SEQ ID NO: 53 - protein, 16 aa, synthetic construct
  REGION 1..16: note = Artificial construct
RSSQNIVHSEGQTYLE

SEQ ID NO: 54 - protein, 16 aa, synthetic construct
  REGION 1..16: note = Artificial construct
RSSQNIVHSNANTYLE

SEQ ID NO: 55 - protein, 16 aa, synthetic construct
  REGION 1..16: note = Artificial construct
RSSQNIVHSNGQTYLE

SEQ ID NO: 56 - protein, 16 aa, synthetic construct
  REGION 1..16: note = Artificial construct
RSSQNIVHSNAQTYLE

SEQ ID NO: 57 - protein, 5 aa, synthetic construct
  REGION 1..5: note = Artificial construct
SYWMH

SEQ ID NO: 58 - protein, 17 aa, synthetic construct
  REGION 1..17: note = Artificial construct
HIYLGGGATNYAEKFRS

SEQ ID NO: 59 - protein, 10 aa, synthetic construct
  REGION 1..10: note = Artificial construct
SQPYYYGMDS

SEQ ID NO: 60 - protein, 11 aa, synthetic construct
  REGION 1..11: note = Artificial construct
RASQDISNYLN

SEQ ID NO: 61 - protein, 7 aa, synthetic construct
  REGION 1..7: note = Artificial construct
YKSRLHS

SEQ ID NO: 62 - protein, 9 aa, synthetic construct
  REGION 1..9: note = Artificial construct
HQGHTIPFT

SEQ ID NO: 63 - protein, 121 aa, synthetic construct
  REGION 1..121: note = Artificial construct
QVQLQQSGPELVKPGASVKMSCKASGYRFSDYVISWVKQRSGQGLEWIGEIYPGDAQTYYNEMFKGRATLTADKSPNTAYMKLSSLTSEDSALYFCARGSYTYDNYAMDFWGQGTSVTVSS

SEQ ID NO: 64 - protein, 121 aa, synthetic construct
  REGION 1..121: note = Artificial construct
QVQLQQSGPELVKPGASVKMSCKASGYRFSDYVISWVKQRSGQGLEWIGEIYPGDANTYYNEMFKGRATLTADKSPNTAYMKLSSLTSEDSALYFCARGSYTYDNYAMDFWGQGTSVTVSS

SEQ ID NO: 65 - protein, 121 aa, synthetic construct
  REGION 1..121: note = Artificial construct
QVQLQQSGPELVKPGASVKMSCKASGYRFSDYVISWVKQRSGQGLEWIGEIYPGEGNTYYNEMFKGRATLTADKSPNTAYMKLSSLTSEDSALYFCARGSYTYDNYAMDFWGQGTSVTVSS

SEQ ID NO: 66 - protein, 112 aa, synthetic construct
  REGION 1..112: note = Artificial construct
DVLMTQTPLSLPVSLGDQASISCRSSQNIVHSEGQTYLEWYLQKPGQSPKLLIYKVSNRFSGVPDRFSGSGSGTDFTLKISRVEAEDLGLYYCFQGSHIPWTFGGGTKLEIK

SEQ ID NO: 67 - protein, 112 aa, synthetic construct
  REGION 1..112: note = Artificial construct
DVLMTQTPLSLPVSLGDQASISCRSSQNIVHSNANTYLEWYLQKPGQSPKLLIYKVSNRFSGVPDRFSGSGSGTDFTLKISRVEAEDLGLYYCFQGSHIPWTFGGGTKLEIK

SEQ ID NO: 68 - protein, 112 aa, synthetic construct
  REGION 1..112: note = Artificial construct
DVLMTQTPLSLPVSLGDQASISCRSSQNIVHSNGQTYLEWYLQKPGQSPKLLIYKVSNRFSGVPDRFSGSGSGTDFTLKISRVEAEDLGLYYCFQGSHIPWTFGGGTKLEIK

SEQ ID NO: 69 - protein, 112 aa, synthetic construct
  REGION 1..112: note = Artificial construct
DVLMTQTPLSLPVSLGDQASISCRSSQNIVHSNAQTYLEWYLQKPGQSPKLLIYKVSNRFSGVPDRFSGSGSGTDFTLKISRVEAEDLGLYYCFQGSHIPWTFGGGTKLEIK

SEQ ID NO: 70 - protein, 330 aa, synthetic construct
  REGION 1..330: note = Artificial construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0002594 A1
(43) Pub. Date: Jan. 2, 2025

(54) ANTI-CD122 ANTIBODIES, ANTI-CD132 ANTIBODIES, AND RELATED BISPECIFIC BINDING PROTEINS

(71) Applicant: SHANGHAI EPIMAB BIOTHERAPEUTICS CO., LTD., Shanghai (CN)

(72) Inventors: Shiyong GONG, Shanghai (CN); Lini HUANG, Shanghai (CN); Chengbin WU, Shanghai (CN); Danqing WU, Shanghai (CN); Xuan WU, Shanghai (CN); Rui ZHANG, Shanghai (CN)

(21) Appl. No.: 18/706,263

(22) PCT Filed: Oct. 24, 2022
(86) PCT No.: PCT/CN2022/127036

§ 371 Date: Apr. 30, 2024

(30) Foreign Application Priority Data
Nov. 2, 2021 (WO) PCT/CN2021/128208

(51) Int. Cl. C07K 16/28; A61K 39/00; A61P 35/00

(52) U.S. Cl. CPC C07K 16/2866; A61P 35/00; A61K 2039/505; C07K 2317/24; C07K 2317/31; C07K 2317/74; C07K 2317/75; C07K 2317/92

(57) ABSTRACT

Provided are antibodies capable of binding CD122, antibodies capable of binding CD132, and bispecific CD122/CD132 binding proteins in FIT-Ig or duobody format prepared with antigen binding fragments thereof. The antibodies and bispecific binding proteins are useful for treating or preventing diseases such as T cell dysfunctional disorders or cancers.

### TECHNICAL FIELD

The present disclosure relates to antibodies capable of binding CD122, antibodies capable of binding CD132, and bispecific binding proteins such as bispecific CD122/CD132 binding proteins (e.g., in FIT-Ig and duobody formats). The antibodies and bispecific binding proteins disclosed herein may be useful for treating or preventing diseases such as T cell dysfunctional disorders or cancers.

### BACKGROUND ART

IL-2 is a pleiotropic cytokine which regulates different immune cells including T cells and natural killer (NK) cells. Two different receptor complexes of IL-2 were identified on different cell types: a high affinity (K_{D}˜10 pM) IL-2 receptor complex composed of alpha chain (IL-2Rα, also known as CD25), beta chain (IL-2Rβ, also known as CD122) and gamma chain (IL-2Rγ, also known as CD132); an intermediate affinity (K_{D}˜1 nM) receptor composed of CD122 and CD132. The high affinity complex is constitutively expressed on immunosuppressive regulatory T cells (Treg) and transiently expressed on activated T cells, while the intermediate affinity receptor is typically expressed on memory-phenotype (MP) CD8+ T cells and NK cells.

Heterodimerization of CD122 and CD132 is considered necessary for effective signal transduction upon binding to IL-2. The signal transduction occurs via several intracellular pathways, including the Janus kinase (JAK)-STAT pathway, the phosphoinositide 3-kinase (PI3K)-AKT pathway, and the mitogen activated protein kinase (MAPK) pathway (Boyman, O. & Sprent, J. Nat. Rev. Immunol. 12, 180-190, 2012). CD25 is not directly involved in IL-2 signaling pathway due to its lack of cytoplasmic kinase activation domain.

Recombinant human IL-2 (rhIL-2) has been developed and approved for the treatment of metastatic melanoma and renal cell carcinoma. But its clinic use is limited by its short half-life and severe adverse effects including vascular leak syndrome (VLS), hypertension, and liver toxicities. According to Krieg et al. (Proc. Natl. Acad. Sci. 107, 11906-11911, 2010), the vascular leak toxicity is related to the expression of the high affinity IL-2 receptor complex on vascular and lung endothelia cells, leading to pulmonary edema. IL-2 induced pulmonary edema could be greatly reduced in vivo by depletion of CD25. On the other hand, utility of IL-2 in cancer therapy could be further compromised by IL-2's preferential binding to the high affinity receptors on Treg cells, which can inhibit the expansion and activation of tumor specific effector T cells thus blunt the anti-tumor efficacy (Sun, Z. et al. Nat. Commun. 10:3874, 2019).

There have been various efforts to engineer rhIL-2 variants that avoid binding to CD25 and extend half-life by PEGylation or other equivalent technologies. Although some of these engineered proteins have the desired functional activity, many of them suffer from high levels of immunogenicity in vivo (Verhoef, J. J. F. et al. Drug Discov. Today 19, 1945-1952 (2014)). Therefore, there is still a need in the field to create an anti-tumor agonist of the IL-2 pathway with desired biological activity and safety profile.

Bispecific antibodies that simultaneously bind IL-2Rβ (CD122) and IL-2Rγ (CD132) but not IL-2Rα (CD25), therefore possess the activity of IL-2 while avoiding IL-2Rα related perturbation are highly desired, as said bispecific antibodies would combine the biological activity of facilitating IL-2Rβ and IL-2Rγ association and downstream signaling with the safety of an antibody molecule.

### SUMMARY OF INVENTION

This disclosure addresses the above needs by providing novel anti-CD122 antibodies, anti-CD132 antibodies, and engineered bispecific proteins that simultaneously bind IL-2Rβ (CD122) and IL-2Rγ (CD132) to induce activation of IL-2R signaling in human immune effector cells without preferentially activating T-regs, shifting the balance towards the activation of effector T-cells and NK-cells.

In particular, in some embodiments, the present disclosure provides anti-CD122 monospecific antibodies, e.g., those with high binding potency to CD122. In some embodiments, the present disclosure also provides monospecific antibodies that bind to CD132, e.g., those that bind to CD132 with high affinity. In some embodiments, the present disclosure also provides a CD122/CD132 bispecific binding protein capable of binding the intermediate affinity IL-2Rβγ receptor consisting of CD122 and CD132 preferentially. In some embodiments, the CD122/CD132 bispecific binding protein is in the format of Fabs-in-Tandem immunoglobulin (FIT-Ig) as described in PCT publication WO2015/103072, or in the format of duobody as described in Labrijn et al., Proc Natl Acad Sci USA. (2013) 110 (13): 5145-50. In some embodiments, the bispecifc multivalent binding proteins described herein are useful to stimulate signaling upon binding to a complex comprising CD122 and CD132, to preferentially stimulate proliferation of effector T cells and/or NK cells over regulatory T cells, and to improve anti-tumor immunity of effector T cells and/or NK cells in vivo and/or in vitro. In some embodiments, the bispecifc multivalent binding proteins described herein are useful to reduce tumor burden/growth/cell expansion.

In some embodiments, the present disclosure also provides methods of making and using the anti-CD122 and anti-CD132 antibodies and CD122/CD132 bispecific binding proteins described herein. Various compositions, e.g., those that may be used in methods of treating or preventing a disorder in an individual that is associated with the impaired functions of effector T cells and/or NK cells and/or the down regulated immune responses, are also disclosed.

### BRIEF DESCRIPTION OF FIGURES

FIG. 1 is a schematic view of FIT-Ig construction.

FIG. 2 is a schematic view of Duobody construction.

FIG. 3 shows CD122/CD132 bispecific antibody FIT2019-86b binds to recombinant CD122 (triangle) and CD132 (diamond) proteins but not to recombinant CD25 (circle) protein.

FIGS. 4A and 4B show that FIT2019-86b (triangle) and Duo2019-86 (inverted triangle) bind to cell surface CD122 (FIG. 4A) and CD132 (FIG. 4B). FIGS. 4C and 4D show that huFIT2019-86b-51 binds to cell surface CD122 (FIG. 4C), and CD132 (FIG. 4D). Irrelevant hIgG was used as negative control.

FIG. 5 shows the activation of CD122/CD132 complex by FIT2019-86b (triangle), but not by Duo2019-86 (inverted triangle).

FIG. 6A shows the activation of pSTAT5 on CD8+ T cells by FIT2019-86b (square) and reference molecules, neo2/15 (diamond), H9 (circle) and hIL-2 (triangle).

FIG. 6B shows the activation of pSTAT5 on CD8+ T cells by huFIT2019-86b-51 (filled circle) and reference molecules, neo2/15 (inverted triangle), H9 (filled square) and hIL-2 (triangle).

FIG. 7A shows the activation of pSTAT5 on Treg cells by FIT2019-86b (square) and reference molecules, neo2/15 (diamond), H9 (circle) and hIL-2 (triangle). FIG. 7B shows the activation of pSTAT5 on Treg cells by huFIT2019-86b-51 (filled circle) and reference molecules, neo2/15 (inverted triangle), H9 (filled square) and hIL-2 (triangle).

FIGS. 8A-8C show the proliferation profile of CD8+ T cells (FIG. 8A), CD4+ T cells (FIG. 8B), and Treg cells (FIG. 8C) upon exposure to FIT2019-86b, huFIT2019-86b-32 and reference molecules, neo2/15, H9 and IL-2 as illustrated under each grouped bars. FIGS. 8D-8F show the proliferation profile of CD8+ T cells (FIG. 8D), CD4+ T cells (FIG. 8E), and Treg cells (FIG. 8F) upon exposure to huFIT2019-86b-51, and reference molecules, neo2/15, H9 and IL-2 as illustrated under each grouped bars. The medium and CD3/CD28 beads were used as controls.

FIG. 9 shows the tumor growth curve of a melanoma/PBMC co-implantation model in immune compromised M-NSG mice treated with FIT2019-86b (inverted triangle) and vehicle control (filled circle).

FIG. 10 shows the body weight change of a melanoma/PBMC co-implantation model in immune compromised M-NSG mice treated with FIT2019-86b (inverted triangle) and vehicle control (filled circle).

FIG. 11 shows the tumor growth curve of a melanoma/PBMC co-implantation model in immune compromised NCG mice treated with huFIT2019-86b-51 in four dosages of 1 mg/kg, 0.3 mg/kg, 0.1 mg/kg and 0.03 mg/kg, and vehicle control.

FIG. 12 shows the body weight change of a melanoma/PBMC co-implantation model in immune compromised NCG mice treated with huFIT2019-86b-51 in four dosages of 1 mg/kg, 0.3 mg/kg, 0.1 mg/kg and 0.03 mg/kg, and vehicle control.

FIG. 13 shows the tumor growth curve of a NSCLC cell/PBMC co-implantation model in immune compromised NCG mice treated with huFIT2019-86b-51 in two dosages of 1 mg/kg and 0.3 mg/kg, and vehicle control.

FIG. 14 shows the body weight change of a NSCLC cell/PBMC co-implantation model in immune compromised NCG mice treated with huFIT2019-86b-51 in two dosages of 1 mg/kg and 0.3 mg/kg, and vehicle control.

FIG. 15 shows the body weight change of mice treated with FIT2019-86b (inverted triangle), IL-2 (square) and vehicle control (circle).

FIG. 16 shows ratio of CD8+ vs. CD4+ T cells in PBMC on day 1 (solid), day 4 (open) and day 7 (horizontal striped) after injection of FIT2019-86b, IL-2 or PBS control. Each group of bars represents data from individual mouse, and all data were normalized to the respective ratio on day 1 of the same mouse.

FIG. 17 shows the ability of huFIT2019-86b-51 and reference molecules to compete with IL2 binding to IL2Rβ. FIG. 17A: injection of IL2Rβ (C4.I-C4.R), IL2Rβ plus huFIT2019-86b-51 (C5.I-C5.R) and running buffer (C6.I-C6.R) on chip immobilized with IL2; FIG. 17B: injection of IL2Rβ (C4.I-C4.R), IL2Rβ plus IL2 (C5.I-C5.R) and running buffer (C6.I-C6.R) on chip immobilized with IL2; FIG. 17C: injection of IL2Rβ (C4.I-C4.R), IL2Rβ plus H9 (C5.I-C5.R) and running buffer (C6.I-C6.R) on chip immobilized with IL2; and FIG. 17D: injection of IL2Rβ (C4.I-C4.R), IL2Rβ plus neo2/15 (C5.I-C5.R) and running buffer (C6.I-C6.R) on chip immobilized with IL2.

### DETAILED DESCRIPTION

This present disclosure pertains to anti-CD122 antibodies, anti-CD132 antibodies, antigen-binding portions thereof, and bispecific binding proteins such as FIT-Igs or duobody that bind to both CD122 and CD132. Various aspects of the present disclosure relate to anti-CD122 and anti-CD132 antibodies and antibody fragments, FIT-Ig and duobody binding proteins that bind to human CD122 and human CD132, and pharmaceutical compositions thereof, as well as nucleic acids, recombinant expression vectors and host cells for making such antibodies, functional antibody fragments, and binding proteins. Methods of using the antibodies, functional antibody fragments, and bispecific binding proteins of the present disclosure to improve the functions of effector T cells and/or NK cells and/or upregulate the immune responses, either in vitro or in vivo; and to treat diseases, especially T cell dysfunctional disorder or cancer, are also encompassed by the present disclosure.

### Definitions

Unless otherwise defined herein, scientific and technical terms used in connection with the present disclosure shall have the meanings that are commonly understood by those of ordinary skill in the art. In the event of any latent ambiguity between the present disclosure and any dictionary or extrinsic definition, definitions provided herein shall prevail. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660 of Kabat et al., 1991) is used for the light chain constant domain CL of kappa and lambda isotype, and the Kabat EU index numbering system (see pages 661-723 of Kabat et al., 1991) is used for the constant heavy chain domains (CH1, Hinge, CH2 and CH3), which is herein further clarified by referring to “numbering according to Kabat EU index” in this case.

General information regarding the sequences of human immunoglobulins light and heavy chains is also given in Kabat et al., 1991.

The term “interleukin-2” or “IL-2” as used herein refers to any native IL-2 from any vertebrate source, including mammals such as primates (e.g. humans) and rodents (e.g. mice and rats), unless otherwise indicated. The term encompasses unprocessed IL-2 as well as any form of IL-2 that results from processing in the cell. Unprocessed human IL-2 additionally comprises an N-terminal 20 amino acid signal peptide, which is absent in the mature IL-2 molecule.

The term “CD25” or “IL-2 receptor a” as used herein refers to any native CD25 from any vertebrate source, including mammals such as primates (e.g. humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g. splice variants or allelic variants. In certain embodiments CD25 is human CD25.

The term “high-affinity IL-2 receptor” as used herein refers to the heterotrimeric form of the IL-2 receptor, consisting of the receptor γ-subunit (also known as common cytokine receptor γ-subunit, γ_{c}, or CD132), the receptor β-subunit (also known as CD122) and the receptor α-subunit (also known as CD25).

The term “intermediate-affinity IL-2 receptor” or “IL-2 receptor By” refers to the IL-2 receptor including only the γ-subunit and the β-subunit, without the α-subunit.

The term “conventional CD4⁺ T cells” refers to CD4⁺ T cells other than regulatory T cells. Conventional CD4⁺ memory T cells are characterized by expression of CD4, CD3, but not FOXP3. “Conventional CD4⁺ memory T cells” are a subset of conventional CD4⁺ T cells, further characterized by lack of expression of CD45RA, in contrast to “conventional CD4⁺ naive T cells” which do express CD45RA.

The term “regulatory T cell” or “Treg cell” refers to a specialized type of CD4 T cell that can suppress the responses of other T cells (effector T cells). Treg cells are characterized by expression of CD4, the α-subunit of the IL-2 receptor (CD25), and the transcription factor forkhead box P3 (FOXP3) (Sakaguchi, Annu Rev Immunol 22, 531-62 (2004)). Treg cells play a critical role in the induction and maintenance of peripheral self-tolerance to antigens, including those expressed by tumors.

“Isolated protein” or “isolated polypeptide” is a protein or polypeptide that by virtue of its origin or source of derivation is not associated with naturally associated components that accompany it in its native state, is substantially free of other proteins from the same species, is expressed by a cell from a different species, or does not occur in nature. A polypeptide that is chemically synthesized or synthesized in a cellular system different from the cell from which it naturally originates may be “isolated” from its naturally associated components. A protein may also be rendered substantially free of naturally associated components by isolation, using protein purification techniques well known in the art.

The term “specific binding” or “specifically binding” in reference to the interaction of an antibody, a binding protein, or a peptide with a second chemical species, means that the interaction is dependent upon the presence of a particular structure (e.g., an antigenic determinant or epitope) on the second chemical species. For example, an antibody recognizes and binds to a specific protein structure rather than to proteins generally. In general, if an antibody is specific for epitope “A”, the presence of a molecule containing epitope A (or free, unlabeled A), in a reaction containing labeled “A” and the antibody, will reduce the amount of labeled A bound to the antibody.

The term “antibody” broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art and non-limiting embodiments are discussed below.

In a full-length antibody, each heavy chain is comprised of a heavy chain variable region (abbreviated herein as VH) and a heavy chain constant region. The heavy chain constant region is comprised of three domains: CH1, CH2, and CH3. Each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDRs), interspersed with regions that are more conserved, termed framework regions (FRs). Each VH and VL is comprised of three CDRs and four FRs, arranged from amino-terminus to carboxy-terminus in the following order: FR1, CDR1, FR2, CDR2, FR3, CDR3, FR4. First, second and third CDRs of a VH domain are commonly enumerated as CDR-H1, CDR-H2, and CDR-H3; likewise, first, second and third CDRs of a VL domain are commonly enumerated as CDR-L1, CDR-L2, and CDR-L3. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, i.e., a CH2 domain and a CH3 domain, and optionally comprises a CH4 domain, for example, as in the case of the Fc regions of IgM and IgE antibodies. The Fc region of IgG, IgA, and IgD antibodies comprises a hinge region, a CH2 domain, and a CH3 domain. In contrast, the Fc region of IgM and IgE antibodies lacks a hinge region but comprises a CH2 domain, a CH3 domain and a CH4 domain. Variant Fc regions having replacements of amino acid residues in the Fc portion to alter antibody effector function are known in the art (see, e.g., Winter et al., U.S. Pat. Nos. 5,648,260 and 5,624,821). The Fc portion of an antibody may mediate one or more effector functions, for example, cytokine induction, ADCC, phagocytosis, complement dependent cytotoxicity (CDC), and/or half-life/clearance rate of antibody and antigen-antibody complexes. In some cases, these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. In still another embodiment at least one amino acid residue is replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region that connects CH1 constant domains to the Fc constant domains (e.g., CH2 and CH3). The anti-inflammatory activity of IgG is dependent on sialylation of the N-linked glycan of the IgG Fc fragment. The precise glycan requirements for anti-inflammatory activity have been determined, such that an appropriate IgG1 Fc fragment can be created, thereby generating a fully recombinant, sialylated IgG1 Fc with greatly enhanced potency (see, Anthony et al., Science, 320:373-376 (2008)).

The terms “antigen-binding portion”, “antigen-binding fragment” and “functional fragment” in context of an antibody are used interchangeably and refer to one or more fragments of an antibody that retain the ability to specifically bind to an antigen, i.e., the same antigen (e.g., CD122, or CD132) as the full-length antibody from which the portion or fragment is derived. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL, and CH1 domains; (ii) a F(ab′)₂ fragment, a bivalent fragment comprising two Fab fragments linked by a disulfide bridge at the hinge region; (iii) an Fd fragment consisting of the VH and CH1 domains; (iv) a Fv fragment consisting of the VL and VH domains of a single arm of an antibody, (v) a dAb fragment (Ward et al., Nature, 341:544-546 (1989); PCT Publication No. WO90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, for example, Bird et al., Science, 242:423-426 (1988); and Huston et al., Proc. Natl. Acad. Sci. USA, 85:5879-5883 (1988)). Such single chain antibodies are also intended to be encompassed within the term “antigen-binding portion” of an antibody and equivalent terms given above. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci. USA, 90:6444-6448 (1993). Such antibody binding portions are known in the art (Kontermann and Dübel eds., Antibody Engineering (Springer-Verlag, New York, 2001), p. 790 (ISBN 3-540-41354-5)). In addition, single chain antibodies also include “linear antibodies” comprising a pair of tandem Fv segments (VH-CH1-VH-CH1) which, together with complementary light chain polypeptides, form a pair of antigen binding regions (Zapata et al., Protein Eng., 8 (10): 1057-1062 (1995); and U.S. Pat. No. 5,641,870)).

An immunoglobulin constant (C) domain refers to a heavy (CH) or light (CL) chain constant domain. Murine and human IgG heavy chain and light chain constant domain amino acid sequences are known in the art.

The term “monoclonal antibody” or “mAb” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic determinant (epitope). Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “human sequence”, in relation to the light chain constant domain CL, heavy chain constant domain CH, and Fc region of the antibody or the binding protein according to the present application, means the sequence is of, or from, human immunoglobulin sequence. The human sequence of the present disclosure may be native human sequence, or a variant thereof including one or more (for example, up to 20, 15, 10) amino acid residue changes.

The term “chimeric antibody” refers to antibodies that comprise heavy and light chain variable region sequences from one species and constant region sequences from another species, such as antibodies having murine heavy and light chain variable regions linked to human constant regions.

The term “CDR-grafted antibody” refers to antibodies that comprise heavy and light chain variable region sequences from one species but in which the sequences of one or more of the CDR regions of VH and/or VL are replaced with CDR sequences of another species, such as antibodies having human heavy and light chain variable regions in which one or more of the human CDRs has been replaced with murine CDR sequences.

The term “humanized antibody” refers to antibodies that comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which CDR sequences from a non-human species (e.g., mouse) are introduced into human VH and VL framework sequences. A humanized antibody is an antibody or a variant, derivative, analog or fragment thereof which immunospecifically binds to an antigen of interest and which comprises framework regions and constant regions having substantially the amino acid sequence of a human antibody but complementarity determining regions (CDRs) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody may be selected from any class of immunoglobulins, including IgM, IgG, IgD, IgA and IgE, and any isotype, including without limitation IgG1, IgG2, IgG3, and IgG4. The humanized antibody may comprise sequences from more than one class or isotype, and particular constant domains may be selected to optimize desired effector functions using techniques well known in the art.

The framework and CDR regions of a humanized antibody need not correspond precisely to the parental sequences, e.g., the donor antibody CDR or the acceptor framework may be mutagenized by substitution, insertion and/or deletion of at least one amino acid residue so that the CDR or framework residue at that site does not correspond to either the donor antibody or the consensus framework. In an exemplary embodiment, such mutations, however, will not be extensive. Usually, at least 80%, at least 85%, at least 90%, or at least 95% of the humanized antibody residues will correspond to those of the parental FR and CDR sequences. Back mutation at a particular framework position to restore the same amino acid that appears at that position in the donor antibody is often utilized to preserve a particular loop structure or to correctly orient the CDR sequences for contact with target antigen.

The term “CDR” refers to the complementarity determining regions within antibody variable domain sequences. There are three CDRs in each of the variable regions of the heavy chain and the light chain, which are designated CDR-H1, CDR-H2, CDR-H3, CDR-L1, CDR-L2, and CDR-L3. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Maryland (1987) and (1991)) not only provides an unambiguous residue numbering system applicable to any variable region of an antibody, but also provides precise residue boundaries defining the three CDRs.

The term “Kabat numbering”, in relation to heavy and light chain CDRs of an antibody, which is recognized in the art, refers to a system of numbering amino acid residues which are more variable (i.e., hypervariable) than other amino acid residues in the heavy and light chain variable regions of an antibody or an antigen-binding portion thereof. See, Kabat et al., Ann. NY Acad. Sci., 190:382-391 (1971); and Kabat et al., Sequences of Proteins of Immunological Interest, Fifth Edition, U.S. Department of Health and Human Services, NIH Publication No. 91-3242 (1991).

The growth and analysis of extensive public databases of amino acid sequences of variable heavy and light regions over the past twenty years have led to the understanding of the typical boundaries between framework regions (FRs) and CDR sequences within variable region sequences and have enabled persons skilled in the art to accurately determine the CDRs according to Kabat numbering, Chothia numbering, or other systems. See, e.g., Martin, “Protein Sequence and Structure Analysis of Antibody Variable Domains,” In Kontermann and Dübel, eds., Antibody Engineering (Springer-Verlag, Berlin, 2001), chapter 31, pages 432-433.

The term “multivalent binding protein” denotes a binding protein comprising two or more antigen binding sites. A multivalent binding protein is, in certain cases, engineered to have three or more antigen binding sites, and is generally not a naturally occurring antibody.

The term “bispecific binding protein” (which can be used interchangeably with the term “bispecific antibody”, unless stated otherwise) refers to a binding protein capable of binding two targets of different specificity. FIT-Ig of the present disclosure comprises four antigen binding sites and is typically a tetravalent binding protein. Duobody of the present disclosure comprises two antigen binding sites and is typically a bivalent binding protein. FIT-Ig or duobody according to this disclosure binds both CD122 and CD132 and is bispecific.

A FIT-Ig comprising two long (heavy) V-C-V-C-Fc chain polypeptides and four short (light) V-C chain polypeptides forms a hexamer exhibiting four Fab antigen binding sites (VH-CH1 paired with VL-CL, sometimes notated VH-CH1:VL-CL). Each half of a FIT-Ig comprises a heavy chain polypeptide and two light chain polypeptides, and complementary immunoglobulin pairing of the VH-CH1 and VL-CL elements of the three chains results in two Fab-structured antigen binding sites, arranged in tandem. In the present disclosure, it is preferred that the immunoglobulin domains comprising the Fab elements are directly fused in the heavy chain polypeptide, without the use of interdomain linkers. That is, the N-terminal V-C element of the long (heavy) polypeptide chains is directly fused at its C-terminus to the N-terminus of another V-C element, which in turn is linked to a C-terminal Fc region. In bispecific FIT-Ig molecules, the tandem Fab elements may be reactive with different antigens. Each Fab antigen binding site comprises a heavy chain variable domain and a light chain variable domain with a total of six CDRs per antigen binding site.

A description of the design, expression, and characterization of FIT-Ig molecules is provided in PCT Publication WO2015/103072. An example of such FIT-Ig molecules comprises a heavy chain and two different light chains. The heavy chain comprises the structural formula VL_{A}-CL-VH_{B}-CH1-Fc where CL is directly fused to VH_{B} (namely “Format LH”) or VH_{A}-CH1-VL_{B}-CL-Fc where CH1 is fused directly to VL_{B} (namely “Format HL”), and the two light polypeptide chains of the FIT-Ig correspondingly have the formulas VH_{A}-CH1 and VL_{B}-CL (for “Format LH”) or VL_{A}-CL and VH_{B}-CH1 (for “Format HL”), respectively; wherein VL_{A} is a variable light domain from a parental antibody that binds antigen A, VL_{B} is a variable light domain from a parental antibody that binds antigen B, VH_{A} is a variable heavy domain from a parental antibody that binds antigen A, VH_{B} is a variable heavy domain from a parental antibody that binds antigen B, CL is a light chain constant domain, CH1 is a heavy chain constant domain, and Fc is an immunoglobulin Fc region (e.g., the C-terminal hinge-CH2-CH3 portion of a heavy chain of an IgG1 antibody). In bispecific FIT-Ig embodiments, antigen A and antigen B are different antigens, or different epitopes of the same antigen. In the present disclosure, one of A and B is CD122 and the other is CD132, for example, A is CD122, and B is CD132.

A duobody binding protein of the present disclosure comprises an Fc region modified as described in Labrijn et al., Proc Natl Acad Sci USA. (2013) 110 (13): 5145-50, referred to as ‘Duobody’ format. In some embodiments, one of the CH3 regions comprises a substitution K409R, and the other CH3 region of the Fc region comprises a substitution F405L.

The term “kₒₙ” (also “Kₒₙ”, “kₒₙ”), as used herein, is intended to refer to the on-rate constant for association of a binding protein (e.g., an antibody) to an antigen to form an association complex, e.g., antibody/antigen complex, as is known in the art. The “kₒₙ” also is known by the terms “association rate constant”, or “ka”, as used interchangeably herein. This value indicates the binding rate of an antibody to its target antigen or the rate of complex formation between an antibody and antigen as is shown by the equation below:

Antibody(“Ab”)+Antigen(“Ag”)→Ab-Ag.

The term “k_{off}” (also “Koff”, “koff”), as used herein, is intended to refer to the off-rate constant for dissociation, or “dissociation rate constant”, of a binding protein (e.g., an antibody) from an association complex (e.g., an antibody/antigen complex) as is known in the art. This value indicates the dissociation rate of an antibody from its target antigen or separation of Ab-Ag complex over time into free antibody and antigen as shown by the equation below:

Ab+Ag←Ab-Ag.

The term “K_{D}” (also “K_{d}”), as used herein, is intended to refer to the “equilibrium dissociation constant”, and refers to the value obtained in a titration measurement at equilibrium, or by dividing the dissociation rate constant (k_{off}) by the association rate constant (kₒₙ). The association rate constant (kₒₙ), the dissociation rate constant (k_{off}), and the equilibrium dissociation constant (K_{D}) are used to represent the binding affinity of an antibody to an antigen. Methods for determining association and dissociation rate constants are well known in the art. Using fluorescence-based techniques offers high sensitivity and the ability to examine samples in physiological buffers at equilibrium.

Other experimental approaches and instruments, such as WAVEsystem (grating-coupled interferometry, GCI) assay (Creoptix AG, Switzerland), BIAcore® (biomolecular interaction analysis) assay (BIAcore International AB, Uppsala, Sweden), and the likes can be used. Biolayer interferometry (BLI) using, e.g., the Octet® RED96 system (Pall ForteBio LLC), is another affinity assay technique. Additionally, a KinExA® (Kinetic Exclusion Assay) assay, available from Sapidyne Instruments (Boise, Idaho) can also be used.

The term “isolated nucleic acid” means a polynucleotide (e.g., of genomic, cDNA, or synthetic origin, or some combination thereof) that, by human intervention, is not associated with all or a portion of the polynucleotides with which it is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present disclosure is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, transfection, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), is intended to refer to a cell into which exogenous DNA has been introduced. In an embodiment, the host cell comprises two or more (e.g., multiple) nucleic acids encoding antibodies, such as the host cells described in U.S. Pat. No. 7,262,028, for example. Such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the Kingdoms of life. In another embodiment, eukaryotic cells include protist, fungal, plant and animal cells. In another embodiment, host cells include but are not limited to the prokaryotic cell line Escherichia coli; mammalian cell lines CHO, HEK 293, COS, NSO, SP2 and PER.C6; the insect cell line Sf9; and the fungal cell Saccharomyces cerevisiae.

As used herein, the term “effective amount” refers to the amount of a therapy that is sufficient to reduce or ameliorate the severity and/or duration of a disorder or one or more symptoms thereof; prevent the advancement of a disorder; cause regression of a disorder; prevent the recurrence, development, or progression of one or more symptoms associated with a disorder; detect a disorder; or enhance or improve the prophylactic or therapeutic effect(s) of another therapy (e.g., prophylactic or therapeutic agent).

Antibodies, functional fragments thereof, and binding proteins according to the present disclosure may be purified (for an intended use) by using one or more of a variety of methods and materials available in the art for purifying antibodies and binding proteins. Such methods and materials include, but are not limited to, affinity chromatography (e.g., using resins, particles, or membranes conjugated to Protein A, Protein G, Protein L, or a specific ligand of the antibody, functional fragment thereof, or binding protein), ion exchange chromatography (for example, using ion exchange particles or membranes), hydrophobic interaction chromatography (“HIC”; for example, using hydrophobic particles or membranes), ultrafiltration, nanofiltration, diafiltration, size exclusion chromatography (“SEC”), low pH treatment (to inactivate contaminating viruses), and combinations thereof, to obtain an acceptable purity for an intended use. A non-limiting example of a low pH treatment to inactivate contaminating viruses comprises reducing the pH of a solution or suspension comprising an antibody, functional fragment thereof, or binding protein of the present disclosure to pH 3.5 with 0.5 M phosphoric acid, at 18° C.-25° C., for 60 to 70 minutes.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual, 2nd ed. (Cold Spring Harbor Laboratory Press, Cold Spring Harbor, N.Y., 1989).

### Anti-CD122 and Anti-CD132 Monospecific Antibodies

Anti-CD122 and anti-CD 132 antibodies of the present disclosure may be produced by any of a number of techniques known in the art, for example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the present disclosure in either prokaryotic or eukaryotic host cells, expression of antibodies in eukaryotic cells, for instance, in mammalian host cells, is particularly contemplated, as widely used for assembly and secretion of properly folded and immunologically active antibodies.

In some embodiments, mammalian host cells for expressing the recombinant antibodies of the present disclosure is Chinese Hamster Ovary (CHO cells) (including dhfr CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982)), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells, or further secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present disclosure. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the present disclosure.

In an exemplary system for recombinant expression of an antibody, or antigen-binding portion thereof, of the present disclosure, a recombinant expression vector encoding both the antibody heavy chain and the antibody light chain is introduced into dhfr CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transfected host cells are cultured to allow expression of the antibody heavy and light chains and intact antibody is recovered from the culture medium. Standard molecular biology techniques are used to prepare the recombinant expression vector, transfect the host cells, select for transfectants, culture the host cells and recover the antibody from the culture medium. The present disclosure further provides a method of making a recombinant anti-CD122 or anti-CD132 antibody by culturing a transfected host cell of the present disclosure in a suitable culture medium until a recombinant antibody of the present disclosure is produced. The method can further comprise isolating the recombinant antibody from the culture medium.

### Anti-CD122 Antibodies

In some embodiments, the present disclosure provides proteins that bind to CD122 at its extracellular domain. In some embodiments, the present disclosure discloses an isolated anti-CD122 antibody or antigen-binding fragment thereof that specifically binds to CD122. In a further embodiment, the anti-CD122 antibody or antigen-binding fragment thereof comprises a set of six CDRs, CDR-H1, CDR-H2, CDR-H3, CDR-L1, CDR-L2, and CDR-L3, wherein:
CDR-H1 comprises the sequence of DYVIS (SEQ ID NO: 44);
CDR-H2 comprises the sequence of EIYPGDGNTYYNEMFKG (SEQ ID NO: 45);
CDR-H3 comprises the sequence of GSYTYDNYAMDF (SEQ ID NO: 46);
CDR-L1 comprises the sequence of RSSQNIVHSNGNTYLE (SEQ ID NO: 50);
CDR-L2 comprises the sequence of KVSNRFS (SEQ ID NO: 51); and
CDR-L3 comprises the sequence of FQGSHIPWT (SEQ ID NO: 52),
wherein the CDRs are defined according to Kabat numbering.

In some embodiments, the anti-CD122 antibody or antigen-binding fragment thereof comprises at least one, two, three, four, but not more than five residue modifications in the CDR sequences of SEQ ID NOs: 44-46 and 50-52. The amino acid modifications may be amino acid substitutions, deletions, and/or additions, for instance, conservative substitution.

In one embodiment, an anti-CD122 antibody or antigen-binding fragment thereof according to the present disclosure comprises CDR-H1, CDR-H2, and CDR-H3, of a heavy chain variable domain VH of SEQ ID NO. 3, as well as CDR-L1, CDR-L2, and CDR-L3 of a light chain variable domain VL of SEQ ID NO. 4. The CDRs can be determined by a person skilled in the art using the most widely CDR definition schemes, for example, Kabat, Chothia or IMGT definitions.

In one embodiment, an anti-CD122 antibody or antigen-binding fragment thereof according to the present disclosure comprises a heavy chain variable domain VH and a light chain variable domain VL, wherein:
the VH domain comprises the sequence of SEQ ID NO: 3, or a sequence having at least 80%, 85%, 90%, 91%, 92%, 93%, 94%, 95%, 96%, 97%, 98%, 99% or more identity therewith, and/or
the VL domain comprises the sequence of SEQ ID NO: 4, or a sequence having at least 80%, 85%, 90%, 91%, 92%, 93%, 94%, 95%, 96%, 97%, 98%, 99% or more identity therewith.

In some embodiments, an anti-CD122 antibody comprises a VH sequence having at least 80%, 85%, 90%, 91%, 92%, 93%, 94%, 95%, 96%, 97%, 98%, or 99% identity contains substitutions (e.g., conservative substitutions), insertions, or deletions relative to the reference sequence, while retains the ability to bind to the CD122 with the same or improved binding properties, such as Kd. In certain embodiments, substitutions, insertions, or deletions occur in regions outside the CDRs (i.e., in the FRs). Optionally, the anti-CD122 antibody comprises Q1E mutation as compared with the VH sequence of SEQ ID NO: 3, to eliminate N-terminal pyroglutamate formation. In some embodiments, an anti-CD122 antibody comprises “DG” (Asp-Gly), “NT” (Asn-Thr) and “NG” (Asn-Gly) in CDR-H2 and/or CDR-L1, such as “DG” in CDR-H2 shown as SEQ ID NO: 45, “NT” in CDR-H2 shown as SEQ ID NOs: 45, 48 and 49; “NG” in CDR-L1 shown as SEQ ID NOs: 50 and 55; “NT” in CDR-L1 shown as SEQ ID NOs: 50 and 54; to mitigate post-translational modification (PTM), which may result in heterogeneity during recombinant antibody manufacturing.

In one embodiment, the isolated anti-CD122 antibody or antigen-binding fragment according to the present disclosure is a chimeric antibody or a humanized antibody. In some embodiments, the anti-CD122 antibody or antigen-binding fragment is a humanized antibody.

In some embodiments, the humanized isolated anti-CD122 antibody or antigen-binding fragment according to the present disclosure comprises one or more back mutations at positions in framework regions to improve the binding property. In some embodiments, the VH domain of the humanized anti-CD122 antibody or antigen-binding fragment according to the present disclosure comprises back mutations from human to residues: a Thr at position 28 (28T), a Thr at position 30 (30T), an Arg at position 38 (38R), a Met at position 48 (48M), a Tyr at position 67 (67Y), a Met at position 69 (69M), an Asn at position 72 (72N), a Thr at position 73 (73T) and/or a Val at position 91 (91V), according to Kabat numbering. In one embodiment, the VL domain of the humanized anti-CD122 antibody or antigen-binding fragment according to the present disclosure comprises back mutations from human to residues: a Ser at position 7 (7S), a Phe at position 36 (36F), a Gln at position 37 (37Q) and/or an Arg at position 46 (46R), according to Kabat numbering.

In one embodiment, the isolated anti-CD122 antibody or antigen-binding fragment according to the present disclosure is a humanized antibody, comprising a set of six CDRs, CDR-H1, CDR-H2, CDR-H3, CDR-L1, CDR-L2, and CDR-L3, wherein:
CDR-H1 comprises the sequence of DYVIS (SEQ ID NO: 44);
CDR-H2 comprises the sequence of EIYPGDGNTYYNEMFKG (SEQ ID NO: 45), EIYPGDAQTYYNEMFKG (SEQ ID NO: 47), EIYPGDANTYYNEMFKG (SEQ ID NO: 48), or EIYPGEGNTYYNEMFKG (SEQ ID NO: 49);
CDR-H3 comprises the sequence of GSYTYDNYAMDF (SEQ ID NO: 46);
CDR-L1 comprises the sequence of RSSQNIVHSNGNTYLE (SEQ ID NO: 50), RSSQNIVHSEGQTYLE (SEQ ID NO: 53), RSSQNIVHSNANTYLE (SEQ ID NO: 54), RSSQNIVHSNGQTYLE (SEQ ID NO: 55), RSSQNIVHSNAQTYLE (SEQ ID NO: 56);
CDR-L2 comprises the sequence of KVSNRFS (SEQ ID NO: 51); and
CDR-L3 comprises the sequence of FQGSHIPWT (SEQ ID NO: 52),
wherein the CDRs are defined according to Kabat numbering.

In some embodiments, the isolated anti-CD122 antibody or antigen-binding fragment according to the present disclosure is a humanized antibody, comprising the amino acid sequences of CDR-H1, CDR-H2, and CDR-H3 selected from the group consisting of: (i) SEQ ID NOs: 44, 45, 46; (ii) SEQ ID NOs: 44, 47, 46; (iii) SEQ ID NOs: 44, 48, 46; or (iv) SEQ ID NOs: 44, 49, 46, according to Kabat numbering.

In one embodiment, the isolated anti-CD122 antibody or antigen-binding fragment according to the present disclosure is a humanized antibody, comprising the amino acid sequences of CDR-L1, CDR-L2, and CDR-L3 selected from the group consisting of: (i) SEQ ID NOs: 50, 51, 52; (ii) SEQ ID NOs: 53, 51, 52; (iii) SEQ ID NOs: 54, 51, 52; (iv) SEQ ID NOs: 55, 51, 52, or (i) SEQ ID NOs: 56, 51, 52, according to Kabat numbering.

In one embodiment, the isolated anti-CD122 antibody or antigen-binding fragment according to the present disclosure is a humanized antibody, comprising CDR-H1, CDR-H2, CDR-H3, CDR-L1, CDR-L2, and CDR-L3 of a heavy chain variable domain VH and a light chain variable domain VL, selected from the group consisting of the following VH/VL sequence pairs: SEQ ID NOs: 21/30, 22/30, 23/30, 24/30, 25/30, 26/30, 21/31, 22/31, 23/31, 24/31, 25/31, 26/31, 21/32, 22/32, 23/32, 24/32, 25/32, 26/32, 21/33, 21/34, 21/35, 27/36, 27/37, 27/38, 27/39, 27/40, 21/36, 28/36, 29/36, 27/41, 27/42, 27/43, and 21/41. The CDRs can be determined by a person skilled in the art using the most widely CDR definition schemes, for example, Kabat, Chothia or IMGT definitions.

In some embodiments, the isolated anti-CD122 antibody or antigen-binding fragment according to the present disclosure comprises a combination of VH and VL sequences selected from the group consisting of:

| combination | VH sequence | VL sequence |
|---|---|---|
| 1 | SEQ ID NO: 21 | SEQ ID NO: 30 |
| 2 | SEQ ID NO: 22 | SEQ ID NO: 30 |
| 3 | SEQ ID NO: 23 | SEQ ID NO: 30 |
| 4 | SEQ ID NO: 24 | SEQ ID NO: 30 |
| 5 | SEQ ID NO: 25 | SEQ ID NO: 30 |
| 6 | SEQ ID NO: 26 | SEQ ID NO: 30 |
| 7 | SEQ ID NO: 21 | SEQ ID NO: 31 |
| 8 | SEQ ID NO: 22 | SEQ ID NO: 31 |
| 9 | SEQ ID NO: 23 | SEQ ID NO: 31 |
| 10 | SEQ ID NO: 24 | SEQ ID NO: 31 |
| 11 | SEQ ID NO: 25 | SEQ ID NO: 31 |
| 12 | SEQ ID NO: 26 | SEQ ID NO: 31 |
| 13 | SEQ ID NO: 21 | SEQ ID NO: 32 |
| 14 | SEQ ID NO: 22 | SEQ ID NO: 32 |
| 15 | SEQ ID NO: 23 | SEQ ID NO: 32 |
| 16 | SEQ ID NO: 24 | SEQ ID NO: 32 |
| 17 | SEQ ID NO: 25 | SEQ ID NO: 32 |
| 18 | SEQ ID NO: 26 | SEQ ID NO: 32 |
| 19 | SEQ ID NO: 21 | SEQ ID NO: 33 |
| 20 | SEQ ID NO: 21 | SEQ ID NO: 34 |
| 21 | SEQ ID NO: 21 | SEQ ID NO: 35 |
| 22 | SEQ ID NO: 27 | SEQ ID NO: 36 |
| 23 | SEQ ID NO: 27 | SEQ ID NO: 37 |
| 24 | SEQ ID NO: 27 | SEQ ID NO: 38 |
| 25 | SEQ ID NO: 27 | SEQ ID NO: 39 |
| 26 | SEQ ID NO: 27 | SEQ ID NO: 40 |
| 27 | SEQ ID NO: 21 | SEQ ID NO: 36 |
| 28 | SEQ ID NO: 28 | SEQ ID NO: 36 |
| 29 | SEQ ID NO: 29 | SEQ ID NO: 36 |
| 30 | SEQ ID NO: 27 | SEQ ID NO: 41 |
| 31 | SEQ ID NO: 27 | SEQ ID NO: 42 |
| 32 | SEQ ID NO: 27 | SEQ ID NO: 43 |
| 33 | SEQ ID NO: 21 | SEQ ID NO: 41 |

In some embodiments, the antibody comprises a VH domain comprising or consisting of the sequence of SEQ ID NO: 21, and a VL domain comprising or consisting of the sequence of SEQ ID NO: 41.

In some embodiments of an anti-CD122 antibody or antigen-binding fragment according to the present disclosure, the antibody or antigen-binding fragment comprises an Fc region, which may be a native or a variant Fc region. In particular embodiments, the Fc region is a human Fc region from IgG1, IgG2, IgG3, IgG4, IgA, IgM, IgE, or IgD. Depending on the utility of the antibody, it may be desirable to use a variant Fc region to change (for example, reduce or eliminate) at least one effector function, for example, ADCC and/or CDC. In some embodiments, the present disclosure provides an anti-CD122 antibody or antigen-binding fragment comprising an Fc region with one or more mutation to change at least one effector function, for example, L234A and L235A.

In some embodiments, antigen-binding fragments of an anti-CD122 antibody according to the present disclosure may be for example, Fv, Fab, Fab′, Fab′-SH, F(ab′)₂; diabodies; linear antibodies; or single-chain antibody molecules (e.g. scFv).

In one embodiment, an anti-CD122 antibody described herein or an antigen-binding fragment thereof binds to the CD122 extracellular domain or a portion thereof. In some embodiments, the CD122 is a human CD 122, for example, having Swiss Prot accession number PI 4784, the complete sequence of which has 551 amino acids consisting a signal peptide (residues 1-26), an extracellular domain (residues 27-240), a transmembrane domain (residues 241-265) and a cytoplasmic domain (residues 266-551). An anti-CD122 antibody described herein or an antigen-binding fragment thereof binds to epitopes within the extracellular domain of CD 122. In one embodiment, the antibody binds to CD122 at the same epitope as an antibody with a VH/VL sequence pair of SEQ ID NOs: 3 and 4 (e.g. CD122-mAb77).

In one embodiment, an anti-CD122 antibody described herein or an antigen-binding fragment thereof has a dissociation constant (K_{D}) to CD122 in the nanomolar (10⁻⁷ to 10⁻⁹) range, for example, less than 8×10⁻⁷ M, less than 5×10⁻⁷ M, less than 3×10⁻⁷ M, less than 1×10⁻⁷ M, less than 8×10⁻⁸M, less than 5×10⁻⁸M, less than 3×10⁻⁸ M, less than 2×10⁻⁸ M, less than 1×10⁻⁸M, less than 8×10⁻⁹M, less than 6×10⁻⁹ M, less than 4×10⁻⁹M, less than 2×10⁻⁹ M, or less than 1×10⁻⁹ M.

Some cancers show elevated levels of CD 122 relative to noncancerous tissue of the same type, preferably from the same patient. Therefore, the anti-CD122 antibody described herein or an antigen-binding fragment thereof can be applied to measure CD 122 at the protein level. (e.g., by immunoassay).

### Anti-CD132 Antibodies

In some embodiments, the present disclosure provides proteins that bind to CD132 at its extracellular domain. In some embodiments, the present disclosure discloses an isolated anti-CD132 antibody or antigen-binding fragment thereof that specifically binds to CD132. In a further embodiment, the anti-CD132 antibody or antigen-binding fragment thereof comprises a set of six CDRs, CDR-H1, CDR-H2, CDR-H3, CDR-L1, CDR-L2, and CDR-L3, wherein:
CDR-H1 comprises the sequence of SYWMH (SEQ ID NO: 57);
CDR-H2 comprises the sequence of HIYLGGGATNYAEKFRS (SEQ ID NO: 58);
CDR-H3 comprises the sequence of SQPYYYGMDS (SEQ ID NO: 59);
CDR-L1 comprises t he sequence of RASQDISNYLN (SEQ ID NO: 60);
CDR-L2 comprises the sequence of YKSRLHS (SEQ ID NO: 61); and
CDR-L3 comprises the sequence of HQGHTIPFT (SEQ ID NO: 62),
wherein the CDRs are defined according to Kabat numbering.

In some embodiments, the anti-CD132 antibody or antigen-binding fragment thereof comprises at least one, two, three, four, but not more than five residue modifications in the CDR sequences of SEQ ID NOs: 57-59 and 60-62. The amino acid modifications may be amino acid substitutions, deletions, and/or additions, for instance, conservative substitution.

In one embodiment, an anti-CD132 antibody or antigen-binding fragment thereof according to the present disclosure comprises CDR-H1, CDR-H2, and CDR-H3 of a heavy chain variable domain VH of SEQ ID NO. 5, as well as CDR-L1, CDR-L2, and CDR-L3 of a light chain variable domain VL of SEQ ID NO. 6. The CDRs can be determined by a person skilled in the art using the most widely CDR definition schemes, for example, Kabat, Chothia or IMGT definitions.

In one embodiment, an anti-CD132 antibody or antigen-binding fragment thereof according to the present disclosure comprises a heavy chain variable domain VH and a light chain variable domain VL, wherein:
the VH domain comprises the sequence of SEQ ID NO: 5, or a sequence having at least 80%, 85%, 90%, 91%, 92%, 93%, 94%, 95%, 96%, 97%, 98%, 99% or more identity therewith, and/or
the VL domain comprises the sequence of SEQ ID NO: 6, or a sequence having at least 80%, 85%, 90%, 91%, 92%, 93%, 94%, 95%, 96%, 97%, 98%, 99% or more identity therewith.

In some embodiments, an anti-CD132 antibody comprises a VH sequence having at least 80%, 85%, 90%, 91%, 92%, 93%, 94%, 95%, 96%, 97%, 98%, or 99% identity contains substitutions (e.g., conservative substitutions), insertions, or deletions relative to the reference sequence, while retains the ability to bind to the CD132 with the same or improved binding properties, such as Kd. In certain embodiments, substitutions, insertions, or deletions occur in regions outside the CDRs (i.e., in the FRs). Optionally, the anti-CD132 antibody comprises Q1E mutation as compared with the VH sequence of SEQ ID NO: 5, to eliminate N-terminal pyroglutamate formation.

In one embodiment, the isolated anti-CD132 antibody or antigen-binding fragment according to the present disclosure is a chimeric antibody or a humanized antibody. In some embodiments, the anti-CD132 antibody or antigen-binding fragment is a humanized antibody.

In some embodiments, the humanized isolated anti-CD132 antibody or antigen-binding fragment according to the present disclosure comprises one or more back mutations at positions in framework regions to improve the binding property. In some embodiments, the VH domain of the humanized anti-CD132 antibody or antigen-binding fragment according to the present disclosure comprises back mutations from human to residues: a Thr at position 28 (28T), a Thr at position 30 (30T), an Arg at position 38 (38R), a Met at position 48 (48M), a Tyr at position 67 (67Y), a Met at position 69 (69M), an Asn at position 72 (72N), a Thr at position 73 (73T) and/or a Val at position 91 (91V), according to Kabat numbering. In one embodiment, the VL domain of the humanized anti-CD132 antibody or antigen-binding fragment according to the present disclosure comprises back mutations from human to residues: a Glu at position 70 (70E) and/or a Tyr at position 71 (71Y), according to Kabat numbering.

In one embodiment, the isolated anti-CD132 antibody or antigen-binding fragment according to the present disclosure is a humanized antibody, comprising a set of six CDRs of above SEQ ID NOs: 57-59 and 60-62.

In one embodiment, the isolated anti-CD132 antibody or antigen-binding fragment according to the present disclosure is a humanized antibody, comprising CDR-H1, CDR-H2, CDR-H3, CDR-L1, CDR-L2, and CDR-L3 of a heavy chain variable domain VH and a light chain variable domain VL, selected from the group consisting of the following VH/VL sequence pairs: SEQ ID NOs: 14/19, 15/19, 16/19, 17/19, 18/19, 14/20, 15/20, 16/20, 17/20, and 18/20. The CDRs can be determined by a person skilled in the art using the most widely CDR definition schemes, for example, Kabat, Chothia or IMGT definitions.

In some embodiments, the isolated anti-CD132 antibody or antigen-binding fragment according to the present disclosure comprises a combination of VH and VL sequences selected from the group consisting of:

| combination | VH sequence | VL sequence |
|---|---|---|
| 34 | SEQ ID NO: 14 | SEQ ID NO: 19 |
| 35 | SEQ ID NO: 15 | SEQ ID NO: 19 |
| 36 | SEQ ID NO: 16 | SEQ ID NO: 19 |
| 37 | SEQ ID NO: 17 | SEQ ID NO: 19 |
| 38 | SEQ ID NO: 18 | SEQ ID NO: 19 |
| 39 | SEQ ID NO: 14 | SEQ ID NO: 20 |
| 40 | SEQ ID NO: 15 | SEQ ID NO: 20 |
| 41 | SEQ ID NO: 16 | SEQ ID NO: 20 |
| 42 | SEQ ID NO: 17 | SEQ ID NO: 20 |
| 43 | SEQ ID NO: 18 | SEQ ID NO: 20 |

In some embodiments, the antibody comprises a VH domain comprising or consisting of the sequence of SEQ ID NO: 14, and a VL domain comprising or consisting of the sequence of SEQ ID NO: 19.

In some embodiments of an anti-CD132 antibody or antigen-binding fragment according to the present disclosure, the antibody or antigen-binding fragment comprises an Fc region, which may be a native or a variant Fc region. In particular embodiments, the Fc region is a human Fc region from IgG1, IgG2, IgG3, IgG4, IgA, IgM, IgE, or IgD. Depending on the utility of the antibody, it may be desirable to use a variant Fc region to change (for example, reduce or eliminate) at least one effector function, for example, ADCC and/or CDC. In some embodiments, the present disclosure provides an anti-CD132 antibody or antigen-binding fragment comprising an Fc region with one or more mutation to change at least one effector function, for example, L234A and L235A.

In some embodiments, antigen-binding fragments of an anti-CD132 antibody according to the present disclosure may be for example, Fv, Fab, Fab′, Fab′-SH, F(ab′)₂; diabodies; linear antibodies; or single-chain antibody molecules (e.g. scFv).

In one embodiment, an anti-CD132 antibody described herein or an antigen-binding fragment thereof binds to the CD132 extracellular domain or a portion thereof. In some embodiments, the CD132 is a human CD 132, for example, having Swiss Prot accession number P31785, the complete sequence of which has 369 amino acids consisting an signal peptide (residues 1-22), an extracellular domain (residues 23-262), a transmembrane domain (residues 263-283) and a cytoplasmic domain (residues 284-369). An anti-CD132 antibody described herein or an antigen-binding fragment thereof binds to epitopes within the extracellular domain of CD 132. In one embodiment, the antibody binds to CD132 at the same epitope as an antibody with a VH/VL sequence pair of SEQ ID NOs: 5 and 6 (e.g. CD132-mAb17).

In one embodiment, an anti-CD132 antibody described herein or an antigen-binding fragment thereof has a dissociation constant (K_{D})) to CD132 in the nanomolar (10⁻⁷ to 10⁻⁹) range, for example, less than 8×10⁻⁷ M, less than 5×10⁻⁷ M, less than 3×10⁻⁷ M, less than 1×10⁻⁷ M, less than 8×10⁻⁸M, less than 5×10⁻⁸M, less than 3×10⁻⁸ M, less than 2×10⁻⁸ M, less than 1×10⁻⁸ M, less than 8×10⁻⁹M, less than 6×10⁻⁹ M, less than 4×10⁻⁹M, less than 2×10⁻⁹ M, or less than 1×10⁻⁹ M.

### CD122×CD132 Bispecific Binding Proteins

In another aspect, the present disclosure provides CD122/CD132 bispecific binding proteins, for example, Fabs-in-Tandem immunoglobulins (FIT-Ig), or duobody, that are capable of binding to both CD122 and CD132. Each variable domain (VH or VL) in a FIT-Ig or a duobody may be obtained from one or more “parental” monoclonal antibodies that bind one of the target antigens, i.e., CD122 or CD132. FIT-Ig or duobody binding proteins may be produced using variable domain sequences of anti-CD122 and anti-CD132 monoclonal antibodies as disclosed herein. For instance, the parental antibodies are humanized antibodies.

An aspect of the present disclosure pertains to selecting parental antibodies with at least one or more properties desired in the FIT-Ig or the duobody molecule. In one embodiment, the antibody properties are selected from the group consisting of antigen specificity, affinity to antigen, cell binding potency, biological function, epitope recognition, stability, production solubility, efficiency, immunogenicity, pharmacokinetics, bioavailability, tissue cross reactivity, and orthologous antigen binding.

In some embodiments, bispecific FIT-Ig proteins according to the present disclosure are configured without any interdomain peptide linker. Whereas in multivalent engineered immunoglobulin formats having tandem binding sites, it was commonly understood in the field that the adjacent binding sites would interfere with each other unless a flexible linker was used to separate the binding sites spatially. It has been discovered for the CD122/CD132 FIT-Ig of the present disclosure, however, that the arrangement of the immunoglobulin domains according to the chain formulas disclosed herein results in polypeptide chains that are well-expressed in transfected mammalian cells, assemble appropriately, and are secreted as bispecific, multivalent immunoglobulin-like binding proteins that bind the target antigens CD122 and CD132. See, Examples, infra. Moreover, omission of synthetic linker sequences from the binding proteins can avoid the creation of antigenic sites recognizable by mammalian immune systems, and in this way the elimination of linkers decreases possible immunogenicity of the FIT-Igs and leads to a half-life in circulation that is like a natural antibody, that is, the FIT-Igs are not rapidly cleared through immune opsonization and capture in the liver.

In some embodiments, an CD122×CD132 bispecific binding protein according to the present application comprises:
a) a first antigen-binding site that specifically binds CD122; and
b) a second antigen-binding site that specifically binds CD132.

In one embodiment, the bispecific binding proteins as described herein comprise a set of six CDRs, CDR-H1, CDR-H2, CDR-H3, CDR-L1, CDR-L2, and CDR-L3 derived from any anti-CD122 antibody or antigen-binding fragment thereof according to the present application and described herein to form the CD122 binding site of the bispecific binding protein. In some further embodiments, the bispecific binding proteins as described herein comprise a VH/VL pair derived from any anti-CD122 antibody or antigen-binding fragment thereof according to the present application and described herein to form the CD122 binding site of the bispecific binding protein.

In one embodiment, the bispecific binding proteins as described herein further comprise a set of six CDRs, CDR-H1, CDR-H2, CDR-H3, CDR-L1, CDR-L2, and CDR-L3 derived from any anti-CD132 antibody or antigen-binding fragment thereof according to the present application and described herein to form the CD132 binding site of the bispecific binding protein. In some further embodiments, the bispecific binding proteins as described herein comprise a VH/VL pair derived from any anti-CD132 antibody or antigen-binding fragment thereof according to the present application and described herein to form the CD132 binding site of the bispecific binding protein.

In one embodiment, the CD122 binding site and the CD132 binding site in a bispecific CD122/CD132 binding protein according to the present application are humanized, comprising humanized VH/VL sequences, respectively.

### Bispecific FIT-Ig Binding Proteins

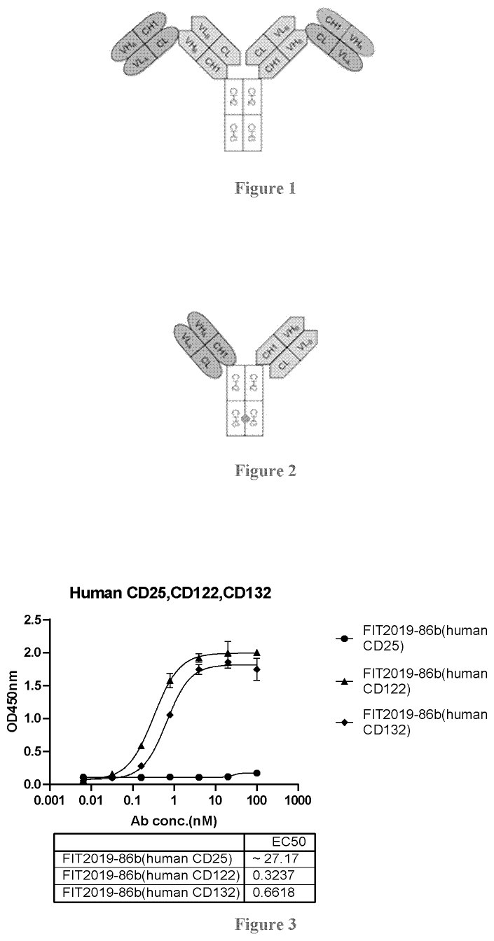

In one embodiment, an CD122×CD132 bispecific binding protein according to the present application is a bispecific FIT-Ig binding protein capable of binding CD122 and CD132. A Fabs-in-Tandem immunoglobulin (FIT-Ig) binding protein is a monomeric, dual-specific, tetravalent binding protein comprising six polypeptide chains, and having four functional Fab binding regions with two outer Fab binding regions and two inner Fab binding regions. As shown in FIG. 1, the binding protein adopts the format (outer Fab-inner Fab-Fc) x2, and binds both antigen A and antigen B. In one aspect, the CD122×CD132 bispecific binding protein according to the present application is a bispecific FIT-Ig binding protein, wherein two Fab domains of the FIT-Ig protein form the first antigen-binding site that specifically binds CD122; and the other two Fab domains of the FIT-Ig protein form the second antigen-binding site that specifically binds CD132. In some embodiments, a FIT-Ig binding protein according to the present disclosure employs no linker between immunoglobulin domains.

In a further embodiment, the present disclosure provides a bispecific Fabs-in-Tandem immunoglobulin (FIT-Ig) binding protein comprises a first polypeptide chain, a second polypeptide chain and a third polypeptide chain, wherein
(i) in Format LH, the first polypeptide chain comprises, from amino terminus to carboxyl terminus, VL_{A}-CL-VH_{B}-CH1-Fc wherein CL is fused directly to VH_{B}; the second polypeptide chain comprises, from amino to carboxyl terminus, VH_{A}-CH1; the third polypeptide chain comprises, from amino to carboxyl terminus, VL_{B}-CL; or (ii) in Format HL, the first polypeptide chain comprises, from amino terminus to carboxyl terminus, VH_{A}-CH1-VL_{B}-CL-Fc wherein CH1 is fused directly to VL_{B}; the second polypeptide chain comprises, from amino to carboxyl terminus, VL_{A}-CL; the third polypeptide chain comprises, from amino to carboxyl terminus, VH_{B}-CH1;

wherein VL is a light chain variable domain, CL is a light chain constant domain, VH is a heavy chain variable domain, CH1 is a heavy chain constant domain, Fc is an immunoglobulin Fc region, for example, the Fc of IgG1 (for instance, the Fc comprising, from amino terminus to carboxyl terminus, hinge-CH2-CH3), wherein VL_{A}-CL pairs with VH_{A}-CH1 to form a first Fab that specifically binds a first antigen A, and VL_{B}-CL pairs with VH_{B}-CH1 to form a second Fab that specifically binds a second antigen B, and wherein the first antigen A is CD122 and the second antigen B is CD132, or wherein the first antigen A is CD132 and the second antigen B is CD122, wherein two of the first polypeptide chains, two of the second polypeptide chains, and two of the third polypeptide chains are associated to form a FIT-Ig binding protein.

In some embodiments of the bispecific FIT-Ig binding protein according the present application, the first polypeptide chain comprises, from amino terminus to carboxyl terminus, VL_{A}-CL-VH_{B}-CH1-Fc, wherein antigen A is CD122 and antigen B is CD132, or antigen A is CD132 and antigen B is CD122.

In some embodiments of the FIT-Ig binding protein, the CD122 binding site is a Fab formed by VL-CL pairing with VH-CH1 (for example, when A is CD122, formed by VL_{A}-CL and VH_{A}-CH1; or when B is CD122, formed by VL_{B}-CL and VH_{B}-CH1), and comprises a set of six CDRs, namely CDR-H1, CDR-H2, CDR-H3, CDR-L1, CDR-L2, and CDR-L3 derived from any anti-CD122 antibody or antigen-binding fragment thereof according to the present application. In some further embodiments, the CDR-H1, CDR-H2, CDR-H3, CDR-L1, CDR-L2, and CDR-L3 comprise respectively the sequences of SEQ ID NO: 44 as CDR-H1, any of SEQ ID NOs: 45, 47-49 as CDR-H2, SEQ ID NO: 46 as CDR-H3 and any of SEQ ID NOs: 50, 53-56 as CDR-L1, SEQ ID NO: 51 as CDR-L2, SEQ ID NO: 52 as CDR-L3.

In some embodiments, the Fab binding to CD122 in the FIT-Ig binding protein comprises a VH/VL pair derived from any anti-CD122 antibody or antigen-binding fragment thereof according to the present application and described herein. In some further embodiments, the VH/VL pair comprises the sequences selected from the group consisting of the following VH/VL sequence pairs: SEQ ID NOs: 3/4, 21/30, 22/30, 23/30, 24/30, 25/30, 26/30, 21/31, 22/31, 23/31, 24/31, 25/31, 26/31, 21/32, 22/32, 23/32, 24/32, 25/32, 26/32, 21/33, 21/34, 21/35, 27/36, 27/37, 27/38, 27/39, 27/40, 21/36, 28/36, 29/36, 27/41, 27/42, 27/43, and 21/41, or sequences having at least 80%, 85%, 90%, 95% or 99% identity therewith. In some embodiments, the Fab binding to CD122 in the FIT-Ig binding protein comprises a VH sequence of SEQ ID NO: 21 and a VL sequence of SEQ ID NO: 41.

In some embodiments of the FIT-Ig binding protein, the CD132 binding site is a Fab formed by VL-CL pairing with VH-CH1 (for example, when A is CD132, formed by VL_{A}-CL and VH_{A}-CH1; or when B is CD132, formed by VL_{B}-CL and VH_{B}-CH1), and comprises a set of six CDRs, namely CDR-H1, CDR-H2, CDR-H3, CDR-L1, CDR-L2, and CDR-L3, derived from any anti-CD132 antibody or antigen-binding fragment thereof according to the present application. In some embodiments, the Fab binding to CD132 formed by VL-CL pairing with VH-CH1 in the FIT-Ig binding protein comprises a set of six CDRs, wherein CDR-H1, CDR-H2, CDR-H3, CDR-L1, CDR-L2, and CDR-L3 comprise the sequences of SEQ ID NOs: 57, 58, 59 and 60, 61, 62 respectively. In some further embodiments, the Fab binding to CD132 comprises a VH/VL pair comprising the sequences of SEQ ID NOs: 22 and 24, or sequences having at least 80%, 85%, 90%, 95% or 99% identity therewith; or the sequences of SEQ ID NOs: 5/6, 14/19, 15/19, 16/19, 17/19, 18/19, 14/20, 15/20, 16/20, 17/20, and 18/20, or sequences having at least 80%, 85%, 90%, 95% or 99% identity therewith.

In some embodiments, the Fab fragments of such FIT-Ig binding proteins incorporate VL_{A}-CL and VH_{A}-CH1 domains from a parental antibody binding to one of the antigens CD122 and CD132, and incorporate VL_{B}-CL and VH_{B}-CH1 domains from a different parental antibody binding to the other of the antigens CD122 and CD132. In some embodiments, Fab moieties in tandem recognizing CD122 and CD132 are respectively formed by VH-CH1:VL-CL pairing.

In accordance with the present disclosure, an CD122/CD132 FIT-Ig binding protein comprises first, second, and third polypeptide chains; wherein the first polypeptide chain comprises, from amino to carboxyl terminus, VL_{CD122}-CL-VH_{CD132}-CH1-hinge-CH2-CH3 wherein CL is directly fused to VH_{CD132}; wherein the second polypeptide chain comprises, from amino to carboxyl terminus, VH_{CD122}-CH1; and wherein the third polypeptide chain comprises, from amino to carboxyl terminus, VL_{CD132}-CL. In alternative embodiments, an CD122/CD132 FIT-Ig binding protein comprises first, second, and third polypeptide chains, wherein the first polypeptide chain comprises, from amino to carboxyl terminus, VH_{CD122}-CH1-VL_{CD132}-CL-hinge-CH2-CH3 wherein CH1 is directly fused to VL_{CD132}, wherein the second polypeptide chain comprises, from amino to carboxyl terminus, VL_{CD122}-CL; and wherein the third polypeptide chain comprises, from amino to carboxyl terminus, VH_{CD132}-CH1. In some embodiments, VL_{CD122} is a light chain variable domain of an anti-CD122 antibody, CL is a light chain constant domain, VH_{CD122} is a heavy chain variable domain of an anti-CD122 antibody, CH1 is a heavy chain constant domain, VL_{CD132} is a light chain variable domain of an anti-CD132 antibody, VH_{CD132} is a heavy chain variable domain of an anti-CD132 antibody; and optionally, the domains VL_{CD132}-CL are the same as the light chain of an anti-CD132 parental antibody, the domains VH_{CD132}-CH1 are the same as the heavy chain variable and heavy chain constant domains of an anti-CD132 parental antibody, the domains VL_{CD122}-CL are the same as the light chain of an anti-CD122 parental antibody, and the domains VH_{CD122}-CH1 are the same as the heavy chain variable and heavy chain constant domains of an anti-CD122 parental antibody.

In the foregoing formulas for a FIT-Ig binding protein, an Fc region may be a native or a variant Fc region. In particular embodiments, the Fc region is a human Fc region from IgG1, IgG2, IgG3, IgG4, IgA, IgM, IgE, or IgD. In particular embodiments, the Fc is a human Fc from IgG1, or a modified human Fc comprising one or more mutations to reduce or eliminate at least one Fc effector function, for example the binding of the Fc to FcγR, ADCC and/or CDC. The mutations may be for example, L234A/L235A (numbering according to Kabat EU index). In one embodiment, the Fc region corresponding to CH1-hinge-CH2-CH3 derived from human constant IgG1 with L234A/L235A mutations has the following amino acid sequence:

(SEQ ID NO. 70)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments of a FIT-Ig binding protein according to the present disclosure, CH1, CL and Fc domains are of or from human sequences. In some embodiments of a FIT-Ig binding protein according to the present disclosure, CH1 is a human IgG1 constant CH1 domain, or a sequence having at least 90%, 95%, 97%, 98%, 99% or more identity herewith. In the foregoing formulas for a FIT-Ig binding protein, CL is a human constant kappa CL domain, or a sequence having at least 90%, 95%, 97%, 98%, 99% or more identity herewith.

In one embodiment, FIT-Ig binding proteins of the present disclosure retain one or more properties of the parental antibodies. In some embodiments, the FIT-Ig retains binding affinity for the target antigens (i.e., CD132 and CD122) comparable to that of the parental antibodies, meaning that the binding affinity of the FIT-Ig binding protein for the CD122 and CD132 antigen targets does not vary by greater than 10-fold in comparison to the binding affinity of the parental antibodies for their respective target antigens, as measured by surface plasmon resonance or biolayer interferometry.

In one embodiment, a FIT-Ig binding protein of the present disclosure binds CD122 and CD132, and is comprised of a first polypeptide chain, a second polypeptide chain, and a third polypeptide chain, wherein:
the first polypeptide chain comprises an amino acid sequence of SEQ ID NO: 7, or a sequence having at least 80%, 85%, 90%, 91%, 92%, 93%, 94%, 95%, 96%, 97%, 98%, 99% or more identity therewith,
the second polypeptide chain comprises an amino acid sequence of SEQ ID NO: 8, or a sequence having at least 80%, 85%, 90%, 91%, 92%, 93%, 94%, 95%, 96%, 97%, 98%, 99% or more identity therewith, and
the third polypeptide chain comprises an amino acid sequence of SEQ ID NO: 9, or a sequence having at least 80%, 85%, 90%, 91%, 92%, 93%, 94%, 95%, 96%, 97%, 98%, 99% or more identity therewith.

In one embodiment, a FIT-Ig binding protein of the present disclosure binds CD122 and CD132, and is comprised of a first polypeptide chain comprising, consisting essentially of, or consisting of the sequence of SEQ ID NO: 7; a second polypeptide chain comprising, consisting essentially of, or consisting of the sequence of SEQ ID NO: 8; and a third polypeptide chain comprising, consisting essentially of, or consisting of the sequence of SEQ ID NO: 9.

### Bispecific Binding Protein Duobody

In one embodiment, an CD122×CD132 bispecific binding protein according to the present application is a duobody capable of binding CD122 and CD132. A duobody shown in FIG. 2 is a monomeric, dual-specific, bi-valent binding protein comprising four polypeptide chains and having two functional Fab binding regions.

A duobody can be generated by mixing two parental antibodies, each containing single matched point mutations in the CH3 domains, and subjecting said two parental antibodies to controlled reducing conditions in vitro that separate the antibodies into HL half-molecules and allow reassembly and reoxidation through a physiological process termed Fab-arm exchange (FAE), in which half-molecules (HL pairs) recombine with half-molecules from other parental antibody molecules, to form highly pure bsAbs.

In some embodiments, the duobody of the present disclosure comprises an Fc region with one of the CH3 regions comprising the substitution K409R, and the other CH3 region of the Fc region comprising the substitution F405L.

In some embodiments, monospecific anti-CD122 IgG1-K409R and anti-CD132 IgG1-F405L antibodies are mixed, then subjected to reduction with 2-MEA for several hours. 2-MEA is removed by dialysis, and the antibodies are left to re-oxidise at 4° C. The fully formed bispecific Duobody is purified by anion exchange chromatography.

In some embodiments, monospecific anti-CD132 IgG1-K409R and anti-CD122 IgG1-F405L antibodies are mixed, then subjected to reduction with 2-MEA for several hours. 2-MEA is removed by dialysis, and the antibodies are left to reoxidise at 4° C. The fully formed bispecific Duobody is purified by anion exchange chromatography.

In some embodiments, the duobody of the present disclosure comprises such HL half-molecules, wherein
(i) One HL half-molecule derived from anti-CD122 antibody of the present disclosure, comprising SEQ ID NO: 44 as CDR-H1, any of SEQ ID NOs: 45, 47-49 as CDR-H2, SEQ ID NO: 46 as CDR-H3 and any of SEQ ID NOs: 50, 53-56 as CDR-L1, SEQ ID NO: 51 as CDR-L2, SEQ ID NO: 52 as CDR-L3. In some embodiments, the HL half-molecule derived from anti-CD122 antibody of the present invention comprises a VH/VL pair derived from any anti-CD122 antibody or antigen-binding fragment thereof according to the present application and described herein. In some further embodiments, the VH/VL pair comprises the sequences selected from the group consisting of the following VH/VL sequence pairs: SEQ ID NOs: 3/4, 21/30, 22/30, 23/30, 24/30, 25/30, 26/30, 21/31, 22/31, 23/31, 24/31, 25/31, 26/31, 21/32, 22/32, 23/32, 24/32, 25/32, 26/32, 21/33, 21/34, 21/35, 27/36, 27/37, 27/38, 27/39, 27/40, 21/36, 28/36, 29/36, 27/41, 27/42, 27/43, and 21/41, or sequences having at least 80%, 85%, 90%, 95% or 99% identity therewith. In some embodiments, the HL half-molecule derived from anti-CD122 antibody comprises a VH sequence of SEQ ID NO: 21 and a VL sequence of SEQ ID NO: 41.
(ii) One HL half-molecule derived from anti-CD132 antibody of the present disclosure, comprising SEQ ID NO: 57 as CDR-H1, SEQ ID NO: 58 as CDR-H2, SEQ ID NO: 59 as CDR-H3 and SEQ ID NO: 60 as CDR-L1, SEQ ID NO: 61 as CDR-L2, SEQ ID NO: 62 as CDR-L3. In some embodiments, the HL half-molecule derived from anti-CD132 antibody of the present disclosure comprises a VH/VL pair derived from any anti-CD132 antibody or antigen-binding fragment thereof according to the present application and described herein. In some further embodiments, the VH/VL pair comprises the sequences selected from the group consisting of the following VH/VL sequence pairs: SEQ ID NOs: 5/6, 14/19, 15/19, 16/19, 17/19, 18/19, 14/20, 15/20, 16/20, 17/20, and 18/20, or sequences having at least 80%, 85%, 90%, 95% or 99% identity therewith. In some embodiments, the HL half-molecule derived from anti-CD132 antibody comprises a VH sequence of SEQ ID NO: 14 and a VL sequence of SEQ ID NO: 19;
(iii) an Fc region with one of the CH3 regions comprising the substitution K409R, and the other CH3 region of the Fc region comprising the substitution F405L.

In one embodiment, a duobody of the present disclosure binds CD122 and CD132 and is comprised of a HL half-molecule derived from anti-CD122 antibody comprising, consisting essentially of, or consisting of the sequence of SEQ ID NO: 12/13; and a HL half-molecule derived from anti-CD132 antibody comprising, consisting essentially of, or consisting of the sequence of SEQ ID NO: 10/11.

### Properties of Bispecific Binding Proteins

In some embodiments, the bispecific binding proteins of the present disclosure are capable of binding to CD122-expressing cells, wherein said cell binding potency is reflected by an EC50 of about 5 nM or lower, 4 nM or lower, 3 nM or lower, 2 nM or lower, or 1 nM or lower, as measured by flow cytometry in a cell-based assay.

In some embodiments, the bispecific binding proteins of the present disclosure are capable of binding to CD132-expressing cells, wherein said cell binding potency is reflected by an EC50 of about 80 nM or lower, 60 nM or lower, 40 nM or lower, 20 nM or lower, or 10 nM or lower, as measured by flow cytometry in a cell-based assay.

In some embodiments, the bispecific binding proteins of the present disclosure bind to human CD122 and human CD132 with a K_{D} of less than about 30 nM, 25 nM, 20 nM, 15 nM, 10 nM, or 5 nM, e.g., as measured by a WAVEsystem or Biacore assay.

In other embodiments, the bispecific binding proteins of the present disclosure bind to cynomolgus CD122 and cynomolgus CD132 with a K_{D} of less than about 100 nM, 80 nM, 60 nM, 40 nM, 20 nM, or 10 nM, e.g., as measured by a WAVEsystem or Biacore assay.

In some embodiments, the bispecific binding proteins of the present disclosure activate intracellular signaling upon contacted with cells expressing a complex comprising CD122 and CD 132 at the cell surface. In some embodiments, the bispecific binding proteins of the present disclosure activate intracellular signaling upon contacted with cells expressing an intermediate-affinity IL-2 receptor at the cell surface. In some embodiments, the bispecific binding proteins of the present disclosure activate intracellular signaling upon contacted with cells expressing a high-affinity IL-2 receptor at the cell surface. The activation of intracellular signaling can be determined by detecting an increase in the level of phosphorylated STAT5 (i.e. pSTAT5). pSTAT5 can be detected, e.g., using reporter-based methods as described in Example 6 and flow cytometry-based STAT5 phosphorylation (pSTAT5) assays as described in Example 7.1.

In some embodiments, the bispecific binding proteins of the present disclosure are capable of increasing the amount of pSTAT5 to more than 1 times, e.g. >1.1 times, >1.2 times, >1.3 times, >1.4 times, >1.5 times, >1.6 times, >1.7 times, >1.8 times, >1.9 times, >2 times, >3 times, >4 times, >5 times, >6 times, >7 times, >8 times, >9 times, >10 times, >20 times, >30 times, >40 times, >50 times, >60 times, >70 times, >80 times, >90 times, or >100 times the level of pSTAT5 detected following culture cells expressing a complex comprising CD122 and CD132 at the cell surface in the absence of the bispecific binding proteins of the present disclosure, or in the presence of a control antigen-binding molecule (e.g. isotype-matched control antigen-binding molecule), in a comparable assay.

In some embodiments, the bispecific binding proteins of the present disclosure are capable of stimulating proliferation of cells expressing CD122 and CD132. Particularly, the bispecific binding proteins of the present disclosure are capable of preferentially stimulating proliferation of effector T cells and/or NK cells over regulatory T cells. In some embodiments, the bispecific binding proteins of the present disclosure preferentially stimulate proliferation of cells expressing an intermediate-affinity IL-2 receptor at the cell surface over cells expressing a high-affinity IL-2 receptor at the cell surface. Cell proliferation can be determined by analyzing cell division over a period of time. Cell division can be analyzed, for example, by CFSE dilution assay as described in Example 7.2.

In some embodiments, the bispecific binding proteins of the present disclosure are capable of increasing the number of proliferating cells to more than 1 times, e.g. >1.1 times, >1.2 times, >1.3 times, >1.4 times, >1.5 times, >1.6 times, >1.7 times, >1.8 times, >1.9 times, >2 times, >3 times, >4 times, >5 times, >6 times, >7 times, >8 times, >9 times, >10 times, >20 times, >30 times, >40 times, >50 times, >60 times, >70 times, >80 times, >90 times, or >100 times the number of proliferating detected following culture cells expressing a complex comprising CD122 and CD132 at the cell surface in the absence of the bispecific binding proteins of the present disclosure, or in the presence of a control antigen-binding molecule (e.g. isotype-matched control antigen-binding molecule), in a comparable assay.

In some embodiments, the bispecific binding proteins of the present disclosure are preferentially stimulates proliferation/expansion of one or more of the following cell types over (i.e. in preference to) regulatory T cells: antigen-specific T cells (e.g. virus-specific T cells), antigen-specific CD4 T cells, antigen-specific CD8 T cells, effector memory CD4 T cells, effector memory CD8 T cells, central memory CD4 T cells, central memory CD8 T cells, cytotoxic CD8+ T cells (i.e. CTLs), NK cells, antigen-specific NK cells, or cells expressing a chimeric antigen receptor (CAR).

In some embodiments, the bispecific binding proteins of the present disclosure increases the number of (i.e. expansion of a population of) cells expressing CD122 and CD132, for example, induces expansion of CD8+ and/or CD4+ T cells, or preferential expansion of CD8+ and/or CD4+ T cells over Treg cells.

### Pharmaceutical Compositions

The present disclosure also provides pharmaceutical compositions comprising an antibody, or antigen-binding portion thereof, or a bispecific multivalent binding protein of the present disclosure (i.e., the primary active ingredient) and a pharmaceutically acceptable carrier. In a specific embodiment, a composition comprises one or more antibodies or binding proteins of the present disclosure. The present disclosure also provides pharmaceutical compositions comprising a combination of anti-CD122 and anti-CD132 antibodies as described herein, or antigen-binding fragment(s) thereof, and a pharmaceutically acceptable carrier. In particular, the present disclosure provides pharmaceutical compositions comprising at least one FIT-Ig binding protein capable of binding CD122 and CD132 and a pharmaceutically acceptable carrier. In particular, the present disclosure provides pharmaceutical compositions comprising at least one duobody binding protein capable of binding CD122 and CD132 and a pharmaceutically acceptable carrier.

Pharmaceutical compositions of the present disclosure may further comprise at least one additional active ingredient. In some embodiments, such an additional ingredient includes, but is not limited to, a prophylactic and/or therapeutic agent, a detection agent, such as an anti-tumor drug, a cytotoxic agent, an antibody of different specificity or functional fragment thereof, a detectable label or reporter. In one embodiment, the pharmaceutical composition comprises one or more additional prophylactic or therapeutic agents, i.e., agents other than the antibodies or binding proteins of the present disclosure, for the treatment or alleviation of a disorder. In one embodiment, the additional prophylactic or therapeutic agents are known to be useful for, have been used, or are currently being used in the prevention, treatment, management, or amelioration of, a disorder or one or more symptoms thereof.

The pharmaceutical compositions comprising proteins of the present disclosure are for use in, but not limited to, diagnosing, detecting, or monitoring a disorder; treating, managing, or ameliorating a disorder or one or more symptoms thereof; and/or research. In some embodiments, the composition may further comprise a carrier, diluent, or excipient. An excipient is generally any compound or combination of compounds that provides a desired feature to a composition other than that of the primary active ingredient (i.e., other than an antibody, functional portion thereof, or binding protein of the present disclosure).

### Nucleic Acid, Vector, and Host Cells

In a further aspect, this disclosure provides isolated nucleic acids encoding one or more amino acid sequences of an anti-CD122 antibody of this disclosure or an antigen-binding fragment thereof; isolated nucleic acids encoding one or more amino acid sequences of an anti-CD132 antibody of this disclosure or an antigen-binding fragment thereof; and isolated nucleic acids encoding one or more amino acid sequences of a bispecific binding protein, including Fabs-in-Tandem immunoglobulin (FIT-Ig) capable of binding both CD122 and CD132. Such nucleic acids may be inserted into a vector for carrying out various genetic analyses or for expressing, characterizing, or improving one or more properties of an antibody or binding protein described herein. A vector may comprise one or more nucleic acid molecules encoding one or more amino acid sequences of an antibody or binding protein described herein in which the one or more nucleic acid molecules is operably linked to appropriate transcriptional and/or translational sequences that permit expression of the antibody or binding protein in a particular host cell carrying the vector. Examples of vectors for cloning or expressing nucleic acids encoding amino acid sequences of binding proteins described herein include, but are not limited to, pcDNA, pTT, pTT3, pEFBOS, pBV, pJV, and pBJ, and derivatives thereof.

The present disclosure also provides a host cell expressing, or capable of expressing, a vector comprising a nucleic acid encoding one or more amino acid sequences of an antibody or binding protein described herein. Host cells useful in the present disclosure may be prokaryotic or eukaryotic. An exemplary prokaryotic host cell is Escherichia coli. Eukaryotic cells useful as host cells in the present disclosure include protist cells, animal cells, plant cells, and fungal cells. An exemplary fungal cell is a yeast cell, including Saccharomyces cerevisiae. An exemplary animal cell useful as a host cell according to the present disclosure includes, but is not limited to, a mammalian cell, an avian cell, and an insect cell. Exemplary mammalian cells include, but are not limited to, CHO cells, HEK cells, and COS cells.

### Methods for Production

In another aspect, the present disclosure provides a method of producing an anti-CD122 antibody or a functional fragment thereof comprising culturing a host cell comprising an expression vector encoding the antibody or functional fragment in culture medium under conditions sufficient to cause the host cell to express the antibody or fragment capable of binding CD122.

In another aspect, the present disclosure provides a method of producing an anti-CD132 antibody or a functional fragment thereof comprising culturing a host cell comprising an expression vector encoding the antibody or functional fragment in culture medium under conditions sufficient to cause the host cell to express the antibody or fragment capable of binding CD132.

In another aspect, the present disclosure provides a method of producing a bispecific, multivalent binding protein capable of binding CD122 and CD132, specifically a FIT-Ig binding protein binding CD122 and CD132, comprising culturing a host cell comprising an expression vector encoding the FIT-Ig binding protein in culture medium under conditions sufficient to cause the host cell to express the binding protein capable of binding CD122 and CD132. The proteins produced by the methods disclosed herein can be isolated and used in various compositions and methods described herein.

### Uses of Antibodies and Binding Proteins

Given their ability to bind to human CD122 and/or CD132, the antibodies described herein, functional fragments thereof, and bispecific multivalent binding proteins described herein can be used to detect CD122 or CD132, or both, e.g., in a biological sample containing cells that express one or both of those target antigens. The antibodies, functional fragments, and binding proteins of the present disclosure can be used in a conventional immunoassay, such as an enzyme linked immunosorbent assay (ELISA), a radioimmunoassay (RIA), or tissue immunohistochemistry. The present disclosure provides a method for detecting CD122 or CD132 in a biological sample comprising contacting a biological sample with an antibody, antigen-binding portion thereof, or binding protein of the present disclosure and detecting whether binding to a target antigen occurs, thereby detecting the presence or absence of the target in the biological sample. The antibody, functional fragment, or binding protein may be directly or indirectly labeled with a detectable substance to facilitate detection of the bound or unbound antibody/fragment/binding protein. Suitable detectable substances include various enzymes, prosthetic groups, fluorescent materials, luminescent materials, and radioactive materials. Examples of suitable enzymes include horseradish peroxidase, alkaline phosphatase, β-galactosidase, or acetylcholinesterase. Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

In some embodiments, the present disclosure provides an antibody or bispecific binding protein of the present disclosure for use in generating/expanding populations of immune cells. In some embodiments, the present disclosure provides an antibody or bispecific binding protein of the present disclosure for use in treating any subject that would benefit from an increase in the number of (i.e. expansion of a population of) cells expressing CD122 and CD132, for example, expansion of CD8+ and/or CD4+ T cells, or preferential expansion of CD8+ and/or CD4+ T cells over Treg cells.

The antibodies (including functional fragments thereof) and binding proteins of the present disclosure can be incorporated into pharmaceutical compositions suitable for administration to a subject. Typically, the pharmaceutical composition comprises an antibody or binding protein of the present disclosure and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols (such as, mannitol or sorbitol), or sodium chloride in the composition. Pharmaceutically acceptable carriers may further comprise minor amounts of auxiliary substances such as wetting or emulsifying agents, preservatives, or buffers, which enhance the shelf life or effectiveness of the antibody or binding protein present in the composition. A pharmaceutical composition of the present disclosure is formulated to be compatible with its intended route of administration.

The method of the present disclosure may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the primary active ingredient may be in powder form for constitution with a suitable vehicle (e.g., sterile pyrogen-free water) before use.

The use of the present disclosure may include administration of compositions formulated as depot preparations. Such long acting formulations may be administered by implantation (e.g., subcutaneously or intramuscularly) or by intramuscular injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

An antibody, functional fragment thereof, or binding protein of the present disclosure also can be administered with one or more additional therapeutic agents useful in the treatment of various diseases. Antibodies, functional fragments thereof, and binding proteins described herein can be used alone or in combination with an additional agent, e.g., an additional therapeutic agent, the additional agent being selected by the skilled artisan for its intended purpose. For example, the additional agent can be a therapeutic agent art-recognized as being useful to treat the disease or condition being treated by the antibody or binding protein of the present disclosure. The additional agent also can be an agent that imparts a beneficial attribute to the therapeutic composition, e.g., an agent that affects the viscosity of the composition.

Having now described the present disclosure in detail, the same will be more clearly understood by reference to the following examples, which are included for purposes of illustration only and are not intended to be limiting of the present disclosure.

### Methods for Treatment and Medical Uses

In some embodiments, the present disclosure provides methods for treating T cell dysfunctional disorders in a subject in need thereof, the method comprising administering to the subject the anti-CD122 antibody and/or the anti-CD132 antibody, or the bispecific, multivalent binding protein capable of binding CD122 and CD132, specifically a FIT-Ig binding protein binding CD122 and CD132, disclosed herein. A T cell dysfunctional disorder may be a disease or condition in which normal T cell function is impaired causing downregulation of the subject's immune response to pathogenic antigens, such as infectious microorganisms, bacteria and viruses.

In some embodiments, the present disclosure provides methods for treating cancers in a subject in need thereof, the method comprising administering to the subject the anti-CD122 antibody and/or the anti-CD132 antibody, or the bispecific, multivalent binding protein capable of binding CD122 and CD132, specifically a FIT-Ig binding protein binding CD122 and CD132, disclosed herein. The cancer to be treated in accordance with the invention described herein may be any unwanted cell proliferation, neoplasm or tumor. The cancer may be benign or malignant and may be primary or secondary. The cancer may be metastatic.

In some embodiments, the cancer to be treated may be a cancer of a tissue selected from the group consisting of colon, rectum, cervix, oropharynx, nasopharynx, liver, stomach, head and neck, oral cavity, oesophagus, lip, mouth, tongue, tonsil, nose, throat, salivary gland, sinus, pharynx, larynx, prostate, lung, bladder, skin, kidney, ovary or mesothelium.

In some embodiments, the cancer to be treated is for example, melanoma, metastatic melanoma, renal cell carcinoma, ovarian cancer, lung cancer, small cell lung cancer, non-small cell lung cancer, brain cancer, head and neck cancer, breast cancer, colon cancer, colorectal cancer, cervical carcinoma, hepatocellular carcinoma, prostate cancer, or bladder cancer.

Methods of treatment described herein may further comprise administering to a subject in need thereof, of additional active ingredient, which is suitably present in combination with the present antibody or binding protein for the treatment purpose intended, for example, another drug having ant-tumor activity. In a method of treatment of the present disclosure, the additional active ingredient may be incorporated into a composition comprising an antibody or binding protein of the present disclosure, and the composition administered to a subject in need of treatment. In another embodiment, a method of treatment of the present disclosure may comprise a step of administering to a subject in need of treatment an antibody or binding protein described herein and a separate step of administering the additional active ingredient to the subject before, concurrently, or after the step of administering to the subject an antibody or binding protein of the present disclosure.

### EXAMPLES

### Example 1. Generation and Characterization of Anti-CD122 and Anti-CD132 Antibodies

Anti-CD122 and anti-CD132 antibodies were obtained respectively by immunizing Balb/c mice with recombinant extracellular domain of human CD122 shown as SEQ ID NO: 1;

HUMAN_CD122_ECD,
SEQ ID NO: 1
AVNGTSQFTCFYNSRANISCVWSQDGALQDTSCQVHAWPDRRRWNQTCEL
LPVSQASWACNLILGAPDSQKLTTVDIVTLRVLCREGVRWRVMAIQDEKP
FENLRLMAPISLQVVHVETHRCNISWEISQASHYFERHLEFEARTLSPGH
TWEEAPLLTLKQKQEWICLETLTPDTQYEFQVRVKPLQGEFTTWSPWSQP
LAFRTKPAALGKD

or with recombinant extracellular domain of human CD132 shown as SEQ ID NO: 2 below:

HUMAN_CD132_ECD,
SEQ ID NO: 2
LNTTILTPNGNEDTTADFFLTTMPTDSLSVSTLPLPEVQCFVENVEYMNC
TWNSSSEPQPTNLTLHYWYKNSDNDKVQKCSHYLFSEEITSGCQLQKKEI
HLYQTFVVQLQDPREPRRQATQMLKLQNLVIPWAPENLTLHKLSESQLEL
NWNNRFLNHCLEHLVQYRTDWDHSWTEQSVDYRHKFSLPSVDGQKRYTFR
VRSRENPLCGSAQHWSEWSHPIHWGSNTSKENPFLFALEA

Mice were immunized at 2-week intervals and monitored for serum titer once a week after the second injection. After 4 immunizations, splenocytes were harvested and fused with mouse myeloma cells to form hybridoma cell lines. Fusion products were plated in selection media containing hypoxanthine-aminopterin-thymidine (HAT) in 96-well plates at 1×10⁵ spleen cells per well. Seven to ten days post-fusion, macroscopic hybridoma colonies were observed. Supernatants of hybridoma cells were then screened and selected to identify cell lines producing CD122-specific mouse antibodies or CD132-specific mouse antibodies.

Upon preliminary characterization of the antigen-specific binding to CD122 or CD132, an anti-CD122 hybridoma cell line named clone #CD122-mAb77 and an anti-CD132 hybridoma cell line named clone #CD132-mAb17 were selected. The variable domain sequences of the two mouse antibodies are set out in Table 1. Complementarity determining regions (CDRs) are underlined based on Kabat numbering.

TABLE 1
Variable domain sequences of anti-CD122 and
anti-CD132 antibodies

| | | SEQ | |
|---|---|---|---|
| Anti- | | ID | |
| body | domain | NO: | Amino acid sequence |
| CD122- | VH | 3 | QVQLQQSGPELVKPGASVKMSCKASGYRFSD |
| mAb77 | | | YVISWVKQRSGQGLEWIGEIYPGDGNTYYNE |
| | | | MFKGRATLTADKSPNTAYMKLSSLTSEDSALY |
| | | | FCARGSYTYDNYAMDFWGQGTSVTVSS |
| | VL | 4 | DVLMTQTPLSLPVSLGDQASISCRSSQNIVHS |
| | | | NGNTYLEWYLQKPGQSPKLLIYKVSNRFSGV |
| | | | PDRFSGSGSGTDFTLKISRVEAEDLGLYYCFQ |
| | | | GSHIPWTFGGGTKLEIK |
| mAb | VH | 5 | EVQLQQPGSELVRPGASVKLSCKASGYTFTSY |
| CD132- | | | WMHWMKQRPGQGLEWIGHIYLGGGATNYAE |
| mAb17 | | | KFRSKATLTADTSSSTAYMQLSSLTSEDSAVYY |
| | | | CTRSQPYYYGMDSWGQGTSVTVSS |
| | VL | 6 | DIQMTQTPSSLSASLGDRVTISCRASQDISNYL |
| | | | NWYQQKPDGTVKLLIYYKSRLHSGVPSRFSG |
| | | | SGSGTEYSLTINNLEQEDFATYFCHQGHTIPFT |
| | | | FGSGTKLEIK |

Binding affinity and kinetics constants of anti-CD122 monospecific antibody and anti-CD132 monospecific antibody can be determined using grating-coupled interferometry (GCI) on WAVEsystem (Creoptix AG, Switzerland) with a label-free biosensor. Briefly, a PCP WAVEchip (Creoptix AG) is pre-immobilized with goat anti-human IgG Fc antibody to a density of ˜1500 pg/mm² by amine coupling, and used to capture sample antibody to a density of ˜30 pg/mm², then exposes to respective antigen, recombinant human CD122 or CD132, serially diluted in 1X HBS-EP+ buffer (Cytiva, Cat No. BR100669) by injection at 60 μL/min for 250s to assess association, followed by 1200s dissociation. After each round of association and dissociation, WAVEchip surface can be regenerated by subject to pH 1.5 Glycine-HCl buffer injection at 60 μL/min for 120s. Sensorgrams are recorded at 25° C., the data can be analyzed on WAVEcontrol (Creoptix AG) and double referenced by subtracting the signals from blank injections and the reference channel. A Langmuir 1:1 model is used for data fitting.

### Example 2. Construction of Bispecific Antibodies Targeting CD122/CD132 Complex

### 2.1 Generation of Bispecific Anti-CD122/CD132 FIT-Ig

FIT-Ig molecules as depicted in FIG. 1 were constructed following the general procedures described in PCT Publication WO 2015/103072. Each FIT-Ig antibody capable of binding CD122 and CD132 consists of three polypeptide chains having the following structures:
Chain #1 (long chain): VL_{A}-CL-VH_{B}-CH1-hinge-CH2-CH3;
Chain #2 (first short chain): VH_{A}-CH1;
Chain #3 (second short chain): VL_{B}-CL;
   Wherein each chain is from N- to C-terminus, “A” is for antibody A against one antigen selected from CD122 and CD132, “B” is for antibody B against another antigen selected from CD122 and CD132.

Amino acid sequences of a bispecific anti-CD122/CD132 FIT-Ig are exemplified in Table 2.

TABLE 2
Amino acid sequences of anti-CD122/CD132
FIT2019-86b

| | SEQ | |
|---|---|---|
| Poly- | ID | |
| peptide | NO. | Amino acid sequence |
| Long | 7 | DVLMTQTPLSLPVSLGDQASISCRSSQNIVHSNG |
| chain: | | NTYLEWYLQKPGQSPKLLIYKVSNRFSGVPDRFS |
| VL_{A}-CL- | | GSGSGTDFTLKISRVEAEDLGLYYCFQGSHIPWTF |
| VH_{B}-CH1- | | GGGTKLEIKRTVAAPSVFIFPPSDEQLKSGTASVV |
| hinge- | | CLLNNFYPREAKVQWKVDNALQSGNSQESVTE |
| CH2-CH3 | | QDSKDSTYSLSSTLTLSKADYEKHKVYACEVTHQ |
| | | GLSSPVTKSFNRGECEVQLQQPGSELVRPGASVK |
| | | LSCKASGYTFTSYWMHWMKQRPGQGLEWIGHI |
| | | YLGGGATNYAEKFRSKATLTADTSSSTAYMQLSS |
| | | LTSEDSAVYYCTRSQPYYYGMDSWGQGTSVTVS |
| | | SASTKGPSVFPLAPSSKSTSGGTAALGCLVKDYFP |
| | | EPVTVSWNSGALTSGVHTFPAVLQSSGLYSLSSV |
| | | VTVPSSSLGTQTYICNVNHKPSNTKVDKKVEPKS |
| | | CDKTHTCPPCPAPEAAGGPSVFLFPPKPKDTLMIS |
| | | RTPEVTCVVVDVSHEDPEVKFNWYVDGVEVHN |
| | | AKTKPREEQYNSTYRVVSVLTVLHQDWLNGKEY |
| | | KCKVSNKALPAPIEKTISKAKGQPREPQVYTLPPS |
| | | REEMTKNQVSLTCLVKGFYPSDIAVEWESNGQPE |
| | | NNYKTTPPVLDSDGSFFLYSKLTVDKSRWQQGN |
| | | VFSCSVMHEALHNHYTQKSLSLSPGK |
| First | 8 | QVQLQQSGPELVKPGASVKMSCKASGYRFSDYV |
| short | | ISWVKQRSGQGLEWIGEIYPGDGNTYYNEMFKG |
| chain: | | RATLTADKSPNTAYMKLSSLTSEDSALYFCARGS |
| VH_{A}-CH1 | | YTYDNYAMDFWGQGTSVTVSSASTKGPSVFPLA |
| | | PSSKSTSGGTAALGCLVKDYFPEPVTVSWNSGAL |
| | | TSGVHTFPAVLQSSGLYSLSSVVTVPSSSLGTQTYI |
| | | CNVNHKPSNTKVDKKVEPKSC |
| Second | 9 | DIQMTQTPSSLSASLGDRVTISCRASQDISNYLNW |
| short | | YQQKPDGTVKLLIYYKSRLHSGVPSRFSGSGSGT |
| chain: | | EYSLTINNLEQEDFATYFCHQGHTIPFTFGSGTKL |
| VL_{B}-CL | | EIKRTVAAPSVFIFPPSDEQLKSGTASVVCLLNNF |
| | | YPREAKVQWKVDNALQSGNSQESVTEQDSKDS |
| | | TYSLSSTLTLSKADYEKHKVYACEVTHQGLSSPV |
| | | TKSFNRGEC |

To produce said FIT-Ig antibody, plasmid constructs encoding the three polypeptide chains were mixed and co-transfected into HEK293 cells. The cells were cultured for 7 days, the supernatants were harvested and subject to Protein A purification. Purified FIT-Ig protein in the eluant was measured for concentration by A280, and homogeneity by size exclusive chromatography (SEC). The exemplified CD122/CD132 bispecific antibody is named as “FIT2019-86b”

### 2.2 Generation of Bispecific Anti-CD122/CD132 Duobody

A Fab-arm exchange based bispecific “duobody” was generated according to publication (Labrijn, A. F. et al. PNAS, 110, 5145-5150, 2013).

For example, VH and VL of CD132-mAb17 were assembled with human IgG1 constant domains containing F405L mutation in CH3 and human kappa constant domains. VH and VL of CD122-mAb77 were assembled with human IgG1 constant domains containing K409R mutation in CH3 and human kappa constant domains. Amino acid sequences of the four polypeptide chains are shown in Table 3. Table 3. Amino acid sequences of anti-CD122/CD132 Duo2019-86b

| | SEQ | |
|---|---|---|
| Poly- | ID | |
| peptide | NO. | Amino acid sequence |
| CD132- | 10 | EVQLQQPGSELVRPGASVKLSCKASGYTFTSYWM |
| mAb17- | | HWMKQRPGQGLEWIGHIYLGGGATNYAEKFRSK |
| IgG1- | | ATLTADTSSSTAYMQLSSLTSEDSAVYYCTRSQPY |
| F405L | | YYGMDSWGQGTSVTVSSASTKGPSVFPLAPSSKS |
| heavy | | TSGGTAALGCLVKDYFPEPVTVSWNSGALTSGVH |
| chain | | TFPAVLQSSGLYSLSSVVTVPSSSLGTQTYICNVNH |
| | | KPSNTKVDKKVEPKSCDKTHTCPPCPAPEAAGGP |
| | | SVFLFPPKPKDTLMISRTPEVTCVVVDVSHEDPEV |
| | | KFNWYVDGVEVHNAKTKPREEQYNSTYRVVSVL |
| | | TVLHQDWLNGKEYKCKVSNKALPAPIEKTISKAK |
| | | GQPREPQVYTLPPSREEMTKNQVSLTCLVKGFYPS |
| | | DIAVEWESNGQPENNYKTTPPVLDSDGSFLLYSKL |
| | | TVDKSRWQQGNVFSCSVMHEALHNHYTQKSLSL |
| | | SPGK |
| CD132- | 11 | DIQMTQTPSSLSASLGDRVTISCRASQDISNYLNW |
| mAb17- | | YQQKPDGTVKLLIYYKSRLHSGVPSRFSGSGSGTE |
| IgG1- | | YSLTINNLEQEDFATYFCHQGHTIPFTFGSGTKLEI |
| F405L | | KRTVAAPSVFIFPPSDEQLKSGTASVVCLLNNFYP |
| light | | REAKVQWKVDNALQSGNSQESVTEQDSKDSTYS |
| chain | | LSSTLTLSKADYEKHKVYACEVTHQGLSSPVTKSF |
| | | NRGEC |
| CD122- | 12 | QVQLQQSGPELVKPGASVKMSCKASGYRFSDYVI |
| mAb77- | | SWVKQRSGQGLEWIGEIYPGDGNTYYNEMFKGR |
| IgG1- | | ATLTADKSPNTAYMKLSSLTSEDSALYFCARGSYT |
| K409R | | YDNYAMDFWGQGTSVTVSSASTKGPSVFPLAPSS |
| heavy | | KSTSGGTAALGCLVKDYFPEPVTVSWNSGALTSG |
| chain | | VHTFPAVLQSSGLYSLSSVVTVPSSSLGTQTYICNV |
| | | NHKPSNTKVDKKVEPKSCDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALPAPIEKTISK |
| | | AKGQPREPQVYTLPPSREEMTKNQVSLTCLVKGF |
| | | YPSDIAVEWESNGQPENNYKTTPPVLDSDGSFFLY |
| | | SRLTVDKSRWQQGNVFSCSVMHEALHNHYTQKS |
| | | LSLSPGK |
| CD122- | 13 | DVLMTQTPLSLPVSLGDQASISCRSSQNIVHSNGN |
| mAb77- | | TYLEWYLQKPGQSPKLLIYKVSNRFSGVPDRFSG |
| IgG1- | | SGSGTDFTLKISRVEAEDLGLYYCFQGSHIPWTFG |
| K409R | | GGTKLEIKRTVAAPSVFIFPPSDEQLKSGTASVVCL |
| light | | LNNFYPREAKVQWKVDNALQSGNSQESVTEQDS |
| chain | | KDSTYSLSSTLTLSKADYEKHKVYACEVTHQGLS |
| | | SPVTKSFNRGEC |

CD132-mAb17 (such as CD132-mAb17-IgG1-F405L antibody) and CD122-mAb77 (such as CD122-mAb77-IgG1-K409R antibody) were produced respectively. Particularly, Expi293F cells were co-transfected with the corresponding heavy chain vector and light chain vectors, cultured for 7 days, then the supernatant was harvested and subject to Protein A chromatography (MabSelect SuRe™; GE Healthcare), the eluant was dialyzed overnight against PBS and filter-sterilized over 0.2-μM dead-end filters.

Purified IgGs thus obtained were measured for concentration by A280 nm with specific extinction coefficients calculated respectively. High-performance size-exclusion chromatography (HP-SEC) assessments of batches of IgG obtained monomeric purity of at least 94%. Endotoxin levels of IgGs used in vivo were all below 0.1 endotoxin units/mg IgG.

CD132-mAb17-IgG1-F405L and CD122-mAb77-IgG1-K409R antibodies were mixed and incubated with 25 mM 2-mercaptoethylamine (2-MEA, Sigma) at a final concentration of 1 mg/mL per antibody each. The mixtures were incubated at 37° C. for 90 min, then dialyzed overnight against PBS to remove 2-MEA through buffer-exchanging. Samples were stored overnight at 4° C. to allow reoxidation for disulfide bonding and generation of stable 1+1 asymmetric bispecific IgG1 molecules as shown in FIG. 2. The exemplified CD122/CD132 bispecific antibody is named as “Duo2019-86b”.

### Example 3. Generation and Assessment of Humanized Anti-CD122/CD132 FIT-Ig

### 3.1 Humanization of Anti-CD122 and Anti-CD132 Antibodies

### 3.1.1 Humanization of Anti-CD132 Antibody

The sequences of mAb CD132-mAb17 variable region in Table 1 were employed to create humanized antibody utilizing available human sequences from V BASE database (https://www2.mrc-lmb.cam.ac.uk/vbase/alignments2.php) for best-matching human germline IgV-gene sequences and framework sequence of highest homologous. For light chain, CDR-L1, CDR-L2, and CDR-L3 of CD132-mAb17 VL (underlined in SEQ ID NO: 6, Table 1) were grafted onto framework sequences of the 012 gene (the closest human V-gene match for VL sequence of SEQ ID NO: 6) with JK2 framework 2 sequence after CDR-L3. For heavy chain, CDR-H1, CDR-H2, and CDR-H3 of CD132-mAb17 VH (underlined in SEQ ID NO: 5, Table 1) were grafted onto framework sequences of the VH1-46 (the closest human match for VH sequence of SEQ ID NO: 5) with JH6 framework 2 sequence after CDR-H3. A three-dimensional (3D) Fv model of CD132-mAb17 was generated by homology modeling, which is able to predict the 3D structure of a query protein through the sequence alignment of template proteins. Certain amino acids at framework positions deemed critical to support loop structures or VH/VL interface according to such model were back-mutated to the corresponding mouse residues to minimize perturbation to affinity/activity (as indicated by double underline in Table 4). Potential mutations of V37M, R38K, M48I, R66K, V67A, M69L, R71A and V78A for heavy chain, and D70E and F71Y for light chain (all by Kabat numbering) were identified. According to importance ranking of each backmutation determined by its interaction with CDRs, the most important backmutations were introduced in humanized VH sequence in priority, with more other backmutations followed gradually. Furthermore, Q1E mutation (bold italic in Table 4) was always included to eliminate N-terminal pyroglutamate formation if applicable. Sequences thus designed were shown in Table 4.

TABLE 4
humanized VH/VL design of mAb CD132-mAb17

| | SEQ | |
|---|---|---|
| | ID | |
| Identifier | NO. | Amino acid sequences* |
| CD132- | 14 | EVQLVQSGAEVKKPGASVKVSCKASGYTFT |
| mAb17VH.1 | | SYWMHWVRQAPGQGLEWMGHIYLGGGAT |
| | | NYAEKFRSRVTMTRDTSTSTVYMELSSLRSE |
| | | DTAVYYCARSQPYYYGMDSWGQGTTVTVSS |
| CD132- | 15 | EVQLVQSGAEVKKPGASVKVSCKASGYTFT |
| mAb17VH.1a | | SYWMHWVRQAPGQGLEWMGHIYLGGGAT |
| | | NYAEKFRSRVTLTADTSTSTVYMELSSLRSED |
| | | TAVYYCARSQPYYYGMDSWGQGTTVTVSS |
| CD132- | 16 | EVQLVQSGAEVKKPGASVKVSCKASGYTFT |
| mAb17VH.1b | | SYWMHWVRQAPGQGLEWMGHIYLGGGAT |
| | | NYAEKFRSKATLTADTSTSTVYMELSSLRSED |
| | | TAVYYCARSQPYYYGMDSWGQGTTVTVSS |
| CD132- | 17 | EVQLVQSGAEVKKPGASVKVSCKASGYTFT |
| mAb17VH.1c | | SYWMHWVKQAPGQGLEWIGHIYLGGGATN |
| | | YAEKFRSKATLTADTSTSTVYMELSSLRSEDT |
| | | AVYYCARSQPYYYGMDSWGQGTTVTVSS |
| CD132- | 18 | EVQLVQSGAEVKKPGASVKVSCKASGYTFT |
| mAb17VH.1d | | SYWMHWMKQAPGQGLEWIGHIYLGGGATN |
| | | YAEKFRSKATLTADTSTSTAYMELSSLRSEDT |
| | | AVYYCARSQPYYYGMDSWGQGTTVTVSS |
| CD132- | 19 | DIQMTQSPSSLSASVGDRVTITCRASQDISNY |
| mAb17VK.1 | | LNWYQQKPGKAPKLLIYYKSRLHSGVPSRFS |
| | | GSGSGTDFTLTISSLQPEDFATYYCHQGHTIPF |
| | | TFGQGTKLEIK |
| CD132- | 20 | DIQMTQSPSSLSASVGDRVTITCRASQDISNY |
| mAb17VK.1a | | LNWYQQKPGKAPKLLIYYKSRLHSGVPSRFS |
| | | GSGSGTEYTLTISSLQPEDFATYYCHQGHTIPF |
| | | TFGQGTKLEIK |

*CDRs are underlined; backmutation residues are double underlined; and Q1E mutation in bold italic.

### 3.1.2 Humanization Design of Anti-CD122 Antibodies

The sequences of CD122-mAb77 variable region in Table 1 were employed to create humanized antibody. utilizing available human sequences from V BASE database (https://www2.mrc-lmb.cam.ac.uk/vbase/alignments2.php) for best-matching human germline IgV-gene sequences and framework sequence of highest homologous. For light chain, CDR-L1, CDR-L2, and CDR-L3 of CD122-mAb77 VL (underlined in SEQ ID NO: 4, Table 1) were grafted onto framework 2 of the A17 gene (the closest human V-gene match for VL sequence of SEQ ID NO: 4) with JK4 framework 2 sequence after CDR-L3. For heavy chain, CDR-H1, CDR-H2, and CDR-H3 of CD122-mAb77 VH (underlined in SEQ ID NO: 3, Table 1) were grafted onto framework sequences of the VH1-8 (the closest human match for VH sequence of SEQ ID NO: 3) with JH6 framework 2 sequence after CDR-H3. Certain amino acids at framework positions deemed critical to support loop structures or VH/VL interface according to 3D Fv model analysis by homology modeling were back-mutated to the corresponding mouse residues to minimize perturbation to affinity/activity (as indicated by double underline in Table 5). Potential mutations of R28T, S30T, K38R, I48M, A67V, L69M, D72N, K73T and F91Y for heavy chain, and T7S, Y36F, L37Q and L46R for light chain (all by Kabat numbering) were identified. According to importance ranking of each backmutation determined by its interaction with CDRs, the most important backmutations were introduced in humanized VH sequence in priority, with more other backmutations followed gradually. Furthermore, Q1E mutation (bold italic in Table 5) was always included to eliminate N-terminal pyroglutamate formation if applicable. “DG” (Asp-Gly), “NT” (Asn-Thr) and “NG” (Asn-Gly) in CDR-H2 or CDR-L1 are prone to post-translational modification (PTM), which may result in heterogeneity during recombinant antibody manufacturing. Therefore, VH and VL CDRs containing point mutations (bold and emphasis marked) of NG (Asn-Gly) to NA (Asn-Ala) mutation, DG (Asp-Gly) to DA (Asp-Ala) mutation, DG (Asp-Gly) to EG (Glu-Gly) mutation, NT (Asn-Thr) to QT (Gln-Thr) mutation, were also designed and evaluated. Sequences thus designed were shown in Table 5.

TABLE 5
humanized VH/VL design of CD122-mAb77

| Identifier* | SEQ ID NO. | Amino acid sequences** |
|---|---|---|
| CD122- | 21 | EVQLVQSGAEVKKPGASVKVSCKASGYTFTDY |
| mAb77VH.1 | | VISWVRQATGQGLEWMGEIYPGD [symbol] TYYNEM |
| | | FKGRVTMTRDTSISTAYMELSSLRSEDTAVYYC |
| | | ARGSYTYDNYAMDFWGQGTTVTVSS |
| CD122- | 22 | EVQLVQSGAEVKKPGASVKVSCKASGYTFTDY |
| mAb77VH.1a | | VISWVRQATGQGLEWMGEIYPGD [symbol] TYYNEM |
| | | FKGRVTMTADTSISTAYMELSSLRSEDTAVYYC |
| | | ARGSYTYDNYAMDFWGQGTTVTVSS |
| CD122- | 23 | EVQLVQSGAEVKKPGASVKVSCKASGYRFSDY |
| mAb77VH.1b | | VISWVRQATGQGLEWMGEIYPGD [symbol] TYYNEM |
| | | FKGRVTMTADTSISTAYMELSSLRSEDTAVYYC |
| | | ARGSYTYDNYAMDFWGQGTTVTVSS |
| CD122- | 24 | EVQLVQSGAEVKKPGASVKVSCKASGYTFTDY |
| mAb77VH.1c | | VISWVRQATGQGLEWMGEIYPGD [symbol] TYYNEM |
| | | FKGRVTLTADTSISTAYMELSSLRSEDTAVYYCA |
| | | RGSYTYDNYAMDFWGQGTTVTVSS |
| CD122- | 25 | EVQLVQSGAEVKKPGASVKVSCKASGYTFTDY |
| mAb77VH.1d | | VISWVKQATGQGLEWIGEIYPGD [symbol] TYYNEMF |
| | | KGRATLTADKSISTAYMELSSLRSEDTAVYYCA |
| | | RGSYTYDNYAMDFWGQGTTVTVSS |
| CD122- | 26 | EVQLVQSGAEVKKPGASVKVSCKASGYRFSDY |
| mAb77VH.1e | | VISWVKQATGQGLEWIGEIYPGD [symbol] TYYNEMF |
| | | KGRATLTADKSISTAYMELSSLRSEDTAVYFCAR |
| | | GSYTYDNYAMDFWGQGTTVTVSS |
| CD122- | 27 | EVQLVQSGAEVKKPGASVKVSCKASGYTFTDY |
| mAb77VH.1 | | VISWVRQATGQGLEWMGEIYPGDGNTYYNEM |
| (DGNT) | | FKGRVTMTRDTSISTAYMELSSLRSEDTAVYYC |
| | | ARGSYTYDNYAMDFWGQGTTVTVSS |
| CD122- | 28 | EVQLVQSGAEVKKPGASVKVSCKASGYTFTDY |
| mAb77VH.1 | | VISWVRQATGQGLEWMGEIYPGD [symbol] NTYYNEM |
| (DANT) | | FKGRVTMTRDTSISTAYMELSSLRSEDTAVYYC |
| | | ARGSYTYDNYAMDFWGQGTTVTVSS |
| CD122- | 29 | EVQLVQSGAEVKKPGASVKVSCKASGYTFTDY |
| mAb77VH.1 | | VISWVRQATGQGLEWMGEIYPG [symbol] GNTYYNEM |
| (EGNT) | | FKGRVTMTRDTSISTAYMELSSLRSEDTAVYYC |
| | | ARGSYTYDNYAMDFWGQGTTVTVSS |
| CD122- | 30 | DVVMTQSPLSLPVTLGQPASISCRSSQNIVHS [symbol] G |
| mAb77VK.1 | | [symbol] TYLEWFQQRPGQSPRRLIYKVSNRFSGVPDRF |
| | | SGSGSGTDFTLKISRVEAEDVGVYYCFQGSHIP |
| | | WTFGGGTKVEIK |
| CD122- | 31 | DVVMTQSPLSLPVTLGQPASISCRSSQNIVHS [symbol] G |
| mAb77VK.1a | | [symbol] TYLEWFQQRPGQSPRLLIYKVSNRFSGVPDRF |
| | | SGSGSGTDFTLKISRVEAEDVGVYYCFQGSHIP |
| | | WTFGGGTKVEIK |
| CD122- | 32 | DVVMTQTPLSLPVTLGQPASISCRSSQNIVHS [symbol] G |
| mAb77VK.1b | | [symbol] TYLEWYQQRPGQSPRLLIYKVSNRFSGVPDR |
| | | FSGSGSGTDFTLKISRVEAEDVGVYYCFQGSHIP |
| | | WTFGGGTKVEIK |
| CD122- | 33 | DVVMTQSPLSLPVTLGQPASISCRSSQNIVHS [symbol] G |
| mAb77VK.1c | | [symbol] TYLEWYQQRPGQSPRLLIYKVSNRFSGVPDR |
| | | FSGSGSGTDFTLKISRVEAEDVGVYYCFQGSHIP |
| | | WTFGGGTKVEIK |
| CD122- | 34 | DVVMTQTPLSLPVTLGQPASISCRSSQNIVHS [symbol] G |
| mAb77VK.1d | | [symbol] TYLEWFQQRPGQSPRLLIYKVSNRFSGVPDRF |
| | | SGSGSGTDFTLKISRVEAEDVGVYYCFQGSHIP |
| | | WTFGGGTKVEIK |
| CD122- | 35 | DVVMTQSPLSLPVTLGQPASISCRSSQNIVHS [symbol] G |
| mAb77VK.1e | | [symbol] TYLEWYLQRPGQSPRLLIYKVSNRFSGVPDRF |
| | | SGSGSGTDFTLKISRVEAEDVGVYYCFQGSHIP |
| | | WTFGGGTKVEIK |
| CD122- | 36 | DVVMTQSPLSLPVTLGQPASISCRSSQNIVHSNG |
| mAb77VK.1a | | NTYLEWFQQRPGQSPRLLIYKVSNRFSGVPDRF |
| (NGNT) | | SGSGSGTDFTLKISRVEAEDVGVYYCFQGSHIP |
| | | WTFGGGTKVEIK |
| CD122- | 37 | DVVMTQTPLSLPVTLGQPASISCRSSQNIVHSN |
| mAb77VK.1b | | GNTYLEWYQQRPGQSPRLLIYKVSNRFSGVPD |
| (NGNT) | | RFSGSGSGTDFTLKISRVEAEDVGVYYCFQGSH |
| | | IPWTFGGGTKVEIK |
| CD122- | 38 | DVVMTQSPLSLPVTLGQPASISCRSSQNIVHSNG |
| mAb77VK.1c | | NTYLEWYQQRPGQSPRLLIYKVSNRFSGVPDR |
| (NGNT) | | FSGSGSGTDFTLKISRVEAEDVGVYYCFQGSHIP |
| | | WTFGGGTKVEIK |
| CD122- | 39 | DVVMTQTPLSLPVTLGQPASISCRSSQNIVHSN |
| mAb77VK.1d | | GNTYLEWFQQRPGQSPRLLIYKVSNRFSGVPD |
| (NGNT) | | RFSGSGSGTDFTLKISRVEAEDVGVYYCFQGSH |
| | | IPWTFGGGTKVEIK |
| CD122- | 40 | DVVMTQSPLSLPVTLGQPASISCRSSQNIVHSNG |
| mAb77VK.1e | | NTYLEWYLQRPGQSPRLLIYKVSNRFSGVPDRF |
| (NGNT) | | SGSGSGTDFTLKISRVEAEDVGVYYCFQGSHIP |
| | | WTFGGGTKVEIK |
| CD122- | 41 | DVVMTQSPLSLPVTLGQPASISCRSSQNIVHSN [symbol] |
| mAb77VK.1a | | NTYLEWFQQRPGQSPRLLIYKVSNRFSGVPDRF |
| (NANT) | | SGSGSGTDFTLKISRVEAEDVGVYYCFQGSHIP |
| | | WTFGGGTKVEIK |
| CD122- | 42 | DVVMTQSPLSLPVTLGQPASISCRSSQNIVHSNG |
| mAb77VK.1a | | [symbol] TYLEWFQQRPGQSPRLLIYKVSNRFSGVPDRF |
| (NGQT) | | SGSGSGTDFTLKISRVEAEDVGVYYCFQGSHIP |
| | | WTFGGGTKVEIK |
| CD122- | 43 | DVVMTQSPLSLPVTLGQPASISCRSSQNIVHSN [symbol] |
| mAb77VK.1a | | [symbol] TYLEWFQQRPGQSPRLLIYKVSNRFSGVPDRF |
| (NAQT) | | SGSGSGTDFTLKISRVEAEDVGVYYCFQGSHIP |
| | | WTFGGGTKVEIK |
| CD122- | 63 | QVQLQQSGPELVKPGASVKMSCKASGYRFSDY |
| mAb77VH(DAQT) | | VISWVKQRSGQGLEWIGEIYPGDAQTYYNEMF |
| | | KGRATLTADKSPNTAYMKLSSLTSEDSALYFCA |
| | | RGSYTYDNYAMDFWGQGTSVTVSS |
| CD122- | 64 | QVQLQQSGPELVKPGASVKMSCKASGYRFSDY |
| mAb77VH | | VISWVKQRSGQGLEWIGEIYPGDANTYYNEMF |
| (DANT) | | KGRATLTADKSPNTAYMKLSSLTSEDSALYFCA |
| | | RGSYTYDNYAMDFWGQGTSVTVSS |
| CD122- | 65 | QVQLQQSGPELVKPGASVKMSCKASGYRFSDY |
| mAb77VH | | VISWVKQRSGQGLEWIGEIYPGEGNTYYNEMF |
| (EGNT) | | KGRATLTADKSPNTAYMKLSSLTSEDSALYFCA |
| | | RGSYTYDNYAMDFWGQGTSVTVSS |
| CD122- | 66 | DVLMTQTPLSLPVSLGDQASISCRSSQNIVHSE |
| mAb77VL(EGQT) | | GQTYLEWYLQKPGQSPKLLIYKVSNRFSGVPD |
| | | RFSGSGSGTDFTLKISRVEAEDLGLYYCFQGSHI |
| | | PWTFGGGTKLEIK |
| CD122- | 67 | DVLMTQTPLSLPVSLGDQASISCRSSQNIVHSN |
| mAb77VL | | ANTYLEWYLQKPGQSPKLLIYKVSNRFSGVPD |
| (NANTA) | | RFSGSGSGTDFTLKISRVEAEDLGLYYCFQGSHI |
| | | PWTFGGGTKLEIK |
| CD122- | 68 | DVLMTQTPLSLPVSLGDQASISCRSSQNIVHSN |
| mAb77VL(NGQT) | | GQTYLEWYLQKPGQSPKLLIYKVSNRFSGVPD |
| | | RFSGSGSGTDFTLKISRVEAEDLGLYYCFQGSHI |
| | | PWTFGGGTKLEIK |
| CD122- | 69 | DVLMTQTPLSLPVSLGDQASISCRSSQNIVHSN |
| mAb77VL(NAQT) | | AQTYLEWYLQKPGQSPKLLIYKVSNRFSGVPD |
| | | RFSGSGSGTDFTLKISRVEAEDLGLYYCFQGSHI |
| | | PWTFGGGTKLEIK |

*VH.1 and VK.1 are CDR-grafted VH/VK without backmutations.
**CDRs are underlined; backmutation residues are double underlined;
Q1E mutation in bold italic;
and point mutations in CDRs are in [symbol] [symbol] [symbol] .

### 3.2 Generation and Binding Assessment of Humanized Anti-CD122/132 FIT-Ig

### 3.2.1 Generation of Humanized Anti-CD122/132 FIT-Ig

Following the FIT-Ig molecule design and generation process as described in Example 2.1, humanized FIT-Igs recognizing both CD122 and CD123 were constructed based on the peptide sequences listed in Table 2 while replacing the VH_{A}/VL_{A} therein with humanized anti-CD122 VH/VL sequences designed in Table 5, and the VH_{B}/VL_{B} therein with humanized anti-CD132 VH/VL sequences designed in Table 4. Such replacements created humanized anti-CD122/CD132 FIT-Ig binding proteins listed in Table 6 below.

TABLE 6
FIT-Ig proteins with humanized anti-CD122/CD132 VH/VL

| FIT-Ig Identifier | VH_{CD122} | VL_{CD122} | VH_{CD132} | VL_{CD132} |
|---|---|---|---|---|
| huFIT2019-86b-11 | CD122-mAb77VH.1 | CD122-mAb77VK.1 | CD132-mAb17VH.1 | CD132-mAb17VK.1 |
| huFIT2019-86b-12 | CD122-mAb77VH.1a | CD122-mAb77VK.1 | CD132-mAb17VH.1 | CD132-mAb17VK.1 |
| huFIT2019-86b-13 | CD122-mAb77VH.1b | CD122-mAb77VK.1 | CD132-mAb17VH.1 | CD132-mAb17VK.1 |
| huFIT2019-86b-14 | CD122-mAb77VH.1c | CD122-mAb77VK.1 | CD132-mAb17VH.1 | CD132-mAb17VK.1 |
| huFIT2019-86b-15 | CD122-mAb77VH.1d | CD122-mAb77VK.1 | CD132-mAb17VH.1 | CD132-mAb17VK.1 |
| huFIT2019-86b-16 | CD122-mAb77VH.1e | CD122-mAb77VK.1 | CD132-mAb17VH.1 | CD132-mAb17VK.1 |
| huFIT2019-86b-17 | CD122-mAb77VH.1 | CD122-mAb77VK.1a | CD132-mAb17VH.1 | CD132-mAb17VK.1 |
| huFIT2019-86b-18 | CD122-mAb77VH.1a | CD122-mAb77VK.1a | CD132-mAb17VH.1 | CD132-mAb17VK.1 |
| huFIT2019-86b-19 | CD122-mAb77VH.1b | CD122-mAb77VK.1a | CD132-mAb17VH.1 | CD132-mAb17VK.1 |
| huFIT2019-86b-20 | CD122-mAb77VH.1c | CD122-mAb77VK.1a | CD132-mAb17VH.1 | CD132-mAb17VK.1 |
| huFIT2019-86b-21 | CD122-mAb77VH.1d | CD122-mAb77VK.1a | CD132-mAb17VH.1 | CD132-mAb17VK.1 |
| huFIT2019-86b-22 | CD122-mAb77VH.1e | CD122-mAb77VK.1a | CD132-mAb17VH.1 | CD132-mAb17VK.1 |
| huFIT2019-86b-23 | CD122-mAb77VH.1 | CD122-mAb77VK.1b | CD132-mAb17VH.1 | CD132-mAb17VK.1 |
| huFIT2019-86b-24 | CD122-mAb77VH.1a | CD122-mAb77VK.1b | CD132-mAb17VH.1 | CD132-mAb17VK.1 |
| huFIT2019-86b-25 | CD122-mAb77VH.1b | CD122-mAb77VK.1b | CD132-mAb17VH.1 | CD132-mAb17VK.1 |
| huFIT2019-86b-26 | CD122-mAb77VH.1c | CD122-mAb77VK.1b | CD132-mAb17VH.1 | CD132-mAb17VK.1 |
| huFIT2019-86b-27 | CD122-mAb77VH.1d | CD122-mAb77VK.1b | CD132-mAb17VH.1 | CD132-mAb17VK.1 |
| huFIT2019-86b-28 | CD122-mAb77VH.1e | CD122-mAb77VK.1b | CD132-mAb17VH.1 | CD132-mAb17VK.1 |
| huFIT2019-86b-29 | CD122-mAb77VH.1 | CD122-mAb77VK.1c | CD132-mAb17VH.1 | CD132-mAb17VK.1 |
| huFIT2019-86b-30 | CD122-mAb77VH.1 | CD122-mAb77VK.1d | CD132-mAb17VH.1 | CD132-mAb17VK.1 |
| huFIT2019-86b-31 | CD122-mAb77VH.1 | CD122-mAb77VK.1e | CD132-mAb17VH.1 | CD132-mAb17VK.1 |
| huFIT2019-86b-32 | CD122-mAb77VH.1 (DGNT) | CD122-mAb77VK.1a (NGNT) | CD132-mAb17VH.1 | CD132-mAb17VK.1 |
| huFIT2019-86b-33 | CD122-mAb77VH.1 (DGNT) | CD122-mAb77VK.1b (NGNT) | CD132-mAb17VH.1 | CD132-mAb17VK.1 |
| huFIT2019-86b-34 | CD122-mAb77VH.1 (DGNT) | CD122-mAb77VK.1c (NGNT) | CD132-mAb17VH.1 | CD132-mAb17VK.1 |
| huFIT2019-86b-35 | CD122-mAb77VH.1 (DGNT) | CD122-mAb77VK.1d (NGNT) | CD132-mAb17VH.1 | CD132-mAb17VK.1 |
| huFIT2019-86b-36 | CD122-mAb77VH.1 (DGNT) | CD122-mAb77VK.1e (NGNT) | CD132-mAb17VH.1 | CD132-mAb17VK.1 |
| huFIT2019-86b-45 | CD122-mAb77VH.1 (DAQT) | CD122-mAb77VK.1a (NGNT) | CD132-mAb17VH.1 | CD132-mAb17VK.1 |
| huFIT2019-86b-46 | CD122-mAb77VH.1(DANT) | CD122-mAb77VK.1a(NGNT) | CD132-mAb17VH.1 | CD132-mAb17VK.1 |
| huFIT2019-86b-47 | CD122-mAb77VH.1 (EGNT) | CD122-mAb77VK.1a (NGNT) | CD132-mAb17VH.1 | CD132-mAb17VK.1 |
| huFIT2019-86b-48 | CD122-mAb77VH.1 (DGNT) | CD122-mAb77VK.1a (NANT) | CD132-mAb17VH.1 | CD132-mAb17VK.1 |
| huFIT2019-86b-49 | CD122-mAb77VH.1 (DGNT) | CD122-mAb77VK.1a (NGQT) | CD132-mAb17VH.1 | CD132-mAb17VK.1 |
| huFIT2019-86b-50 | CD122-mAb77VH.1 (DGNT) | CD122-mAb77VK.1a (NAQT) | CD132-mAb17VH.1 | CD132-mAb17VK.1 |
| huFIT2019-86b-51 | CD122-mAb77VH.1 (DAQT) | CD122-mAb77VK.1a (NANT) | CD132-mAb17VH.1 | CD132-mAb17VK.1 |

### 3.2.2 Target Binding Assessment of Humanized CD122/CD132 FIT-Ig

Binding affinity and kinetics constants of humanized CD122/CD132 FIT-Ig were determined using a label-free biosensor on WAVEsystem (Creoptix AG, Switzerland) by grating-coupled interferometry (GCI). Briefly, a PCP WAVEchip (Creoptix AG) was pre-immobilized with goat anti-human IgG Fc antibody to a density of 3830 pg/mm² by amine coupling, and used to capture humanized CD122/CD132 FIT-Ig generated from Example 3.2.1 to a density of ˜90 pg/mm², then exposed to recombinant human/cyno CD122 or CD132 serially diluted (3× dilutions from 200 nM to 823 PM for human CD122, human CD132 and cyno CD132, respectively; and from 1000 nM to 1.37 nM for cyno CD122) in 1X HBS-EP+ buffer (Cytiva, Cat No. BR100669) by injection at 60 μL/min for 250s to assess association, followed by 1200s dissociation. After each round of association and dissociation, WAVEchip surface was regenerated by subject to pH 1.5 Glycine-HCl buffer injection at 60 μL/min for 120s. Sensorgrams were recorded at 25° C., the data were analyzed on WAVEcontrol (Creoptix AG) and double referenced by subtracting the signals from blank injections and the reference channel. A Langmuir 1:1 model was used for data fitting. Kinetics constants obtained thereby for humanized CD122/CD132 FIT-Igs generated from Example 3.2.1 showed that they all bind comparably with high affinity to human and cynomolgus CD122 and CD132. Table 7 exemplified the binding kinetics constants of huFIT2019-86b-51.

TABLE 7
Binding kinetics of huFIT2019-86b-51

| Sample ID | Ka (1/Ms) | Kd (1/s) | K_{D}(M) |
|---|---|---|---|
| Human CD132 | 3.75E+04 | 3.39E−04 | 9.02E−09 |
| Human CD122 | 6.27E+04 | 1.07E−03 | 1.71E−08 |
| Cyno CD132 | 2.70E+04 | 2.86E−04 | 1.06E−08 |
| Cyno CD122 | 7.61E+04 | 6.97E−03 | 9.16E−08 |

### Example 4. CD25 Binding Assessment of CD122/CD132 Bispecific Antibodies

The CD122/CD132 bispecific antibody, FIT2019-86b, was tested for CD25 binding activity by ELISA. In particular, 96-well plates were coated respectively with 1 μg/mL recombinant human CD25, CD122 or CD132 at 4° C. overnight, washed once with washing buffer (PBST, PBS containing 0.05% Tween 20), blocked with blocking buffer (PBST containing 1% BSA) at room temperature for 0.5 hour, added with FIT2019-86b at assigned concentration and incubated at RT for 0.5 hour. Plates were washed three times with PBST, added HRP labeled anti-human IgG secondary antibody and incubated at RT for 15 minutes, then washed 5 times with PBST. For color development, 100 μl of tetramethylbenzidine (TMB) chromogenic solution was added to each well, the reaction was quenched by 1 N HCl and absorbance at 450 nm was measured on a microplate reader. Binding signals were plotted against antibody concentration with GraphPad Prism 6.0 software and EC₅₀ values were calculated accordingly.

As shown in FIG. 3, FIT2019-86b antibody had no binding to CD25, while specific binding activities to CD122 and CD132 targets respectively were observed, with EC₅₀ of 0.3237 nM for the binding of FIT2019-86b antibody to human CD122, and with EC50 of 0.6618 nM for the binding of FIT2019-86b antibody to human CD132.

### Example 5. Cell Surface Binding Characterization of CD122/CD132 Bispecific Antibodies

CD122/CD132 bispecific antibodies named as FIT2019-86b and Duo2019-86b, and humanized anti-CD122/CD132 antibody huFIT2019-86b-51 were measured for cell binding activity using HEK293 cell lines overexpressing human CD122 and human CD132, respectively.

Briefly, a 96-well plate was seeded with 3×10⁵ cells each well, centrifuged at 400 g for 5 minutes and supernatants discarded, then added 100 μl of antibody (5× serial dilutions, 0.0064 to 100 nM) each well and gently mixed. After 60 minutes of incubation at 4° C., plates were washed several times to remove excess antibodies. Secondary fluorochrome-conjugated goat anti-human IgG antibody (Jackson Immunoresearch, Cat No. 109-606-098) was then added and incubated with cells at 4° C. for 20 minutes. After another round of centrifugation and washing, cells were resuspended in assay buffer (1X PBS containing 2% FBS) for reading on a flow-cytometer. Median Fluorescence Intensity (MFI) readouts were plotted against antibody concentration and analyzed with GraphPad Prism 6.0 software.

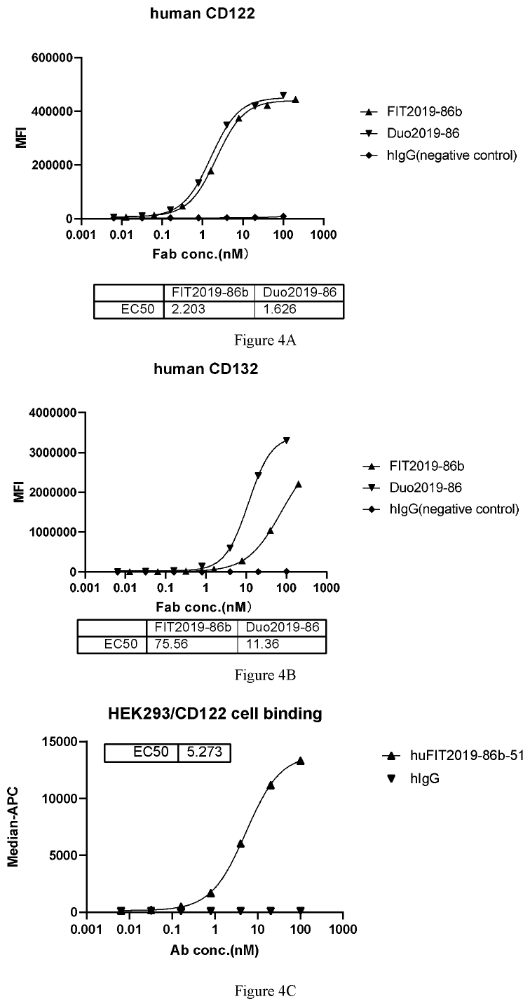

FIG. 4 demonstrates that CD122/CD132 bispecific antibodies FIT2019-86b, Duo2019-86b and huFIT2019-86b-51 exhibited binding activity to both CD122 and CD132 targets expressed on cell surface.

### Example 6. Activation of Signaling Pathway of CD122/CD132 Complex by CD122/CD132 Bispecific Antibodies

A STAT5-inducible secreted alkaline phosphatase (SEAP) reporter gene assay was utilized to assess the activation of signaling pathway of CD122/CD132 complex.

Briefly, 96-well assay plates were added 100 μl antibody (4× serial dilution in DMEM with 10% FBS, 0.000031 nM to 2 nM) and 50,000 cells HEK-Blue™ IL-2 cells (InvivoGen, Cat #hkb-il2) suspension in each well, incubated overnight in tissue culture incubator (37° C., 5% CO2), 20 μL of supernatant were then transferred from each well to another 96-well assay plate and mixed with 180 μL QUANTI-Blue solution (InvivoGen, Cat code rep-qbs). The latter assay plates were incubated at 37° C. for 60 min for color development and measured for absorbance at 630 nm (OD630) on a microplate reader. The OD630 represents level of STAT5 activation.

FIG. 5 illustrates that only FIT-2019-86b induced STAT5 activation (suggests activated CD122/CD132 complex), while Duo2019-86 did not. Considering the same variable regions of anti-CD122 and anti-CD132 antibody used in both formats, the difference of activation potency may be format dependent. The humanized anti-CD122/CD132 FIT-Igs generated from Example 3.2.1 were tested in STAT5-inducible SEAP reporter gene assay (RGA) as well with bispecific FIT2019-86b used as positive control. EC50 values were calculated and in a range of about 0.1 to 0.3 nM. Lower EC50 value indicates better STAT5 activation activity of the antibody. Table 8 shows EC50 values of some exemplified FIT-Igs.

TABLE 8
EC50 from SEAP reporter gene assay of
exemplified anti-CD122/CD132 FIT-Ig

| FIT-Ig identifier | EC50 (nM) |
|---|---|
| FIT2019-86b | 0.1248 |
| huFIT2019-86b-32 | 0.1626 |
| huFIT2019-86b-35 | 0.1627 |
| huFIT2019-86b-45 | 0.1057 |
| huFIT2019-86b-46 | 0.1193 |
| huFIT2019-86b-47 | 0.1214 |
| huFIT2019-86b-48 | 0.1676 |
| huFIT2019-86b-49 | 0.2162 |
| huFIT2019-86b-50 | 0.2857 |
| huFIT2019-86b-51 | 0.1899 |

### Example 7. In Vitro Agonistic Effect of CD122/CD132 FIT-Ig

### 7.1 Induction of STAT5 Phosphorylation in Human Lymphocytes

The ability of FIT2019-86b and huFIT2019-86b-51 to stimulate IL-2R signaling in human CD4+ T-cells, CD8+ T-cells, Treg cells and NK-cells was assessed by a flow cytometry-based STAT5 phosphorylation (pSTAT5) assay.

Briefly, human PBMCs were prepared from frozen LeukoPak. PBMC cells were thawed (if needed), washed twice with RPMI1640 medium and resuspended at 5.5×10⁶ cells/mL, then transferred 90 μL/well to a 96-well plate, added with 10 μL of antibody or reference IL-2/IL-2 variants as indicated (5× serial dilution, 0.00128 nM to 100 nM) and incubated at 37° C. for 15 min. The plate was then sealed and returned to the incubator for an additional 1 hour. Immediately after the second incubation, the cells were fixed with 100 μL pre-warmed BD Cytofix™ Buffer (Cat.No. 554655) for 10 min at 37° C. then centrifuged at 350g for 7 min. Supernatant was discarded and 200 μL pre-chilled BD Phosflow Perm Buffer III (BD, Cat #554655) was added and incubated with cells on ice for 30 min in the dark. The cells were then washed twice by 450g centrifugation at 4° C. for 7 min and resuspension with 200 μL cell staining buffer (Biolegend, Cat #420201). Next, the cell pellet was stained with antibody mixture (anti-CD3, Biolegend, Cat. No. 344818; anti-CD4, Biolegend, Cat. No. 317408; anti-CD8, Biolegend, Cat. No. 565310; anti-CD25, BD, Cat. No. 562442; anti-Foxp3, Biolegend, Cat. No. 560852; anti-pSTAT5, BD, Cat. No. 562076; 2.5 μL of each antibody per sample) and incubated at RT for 60 min in the dark. After staining, the cells were centrifuged again and washed twice. Subsequently, the cell pellet was resuspended in 200 μL cell staining buffer and analyzed with a flow cytometer.

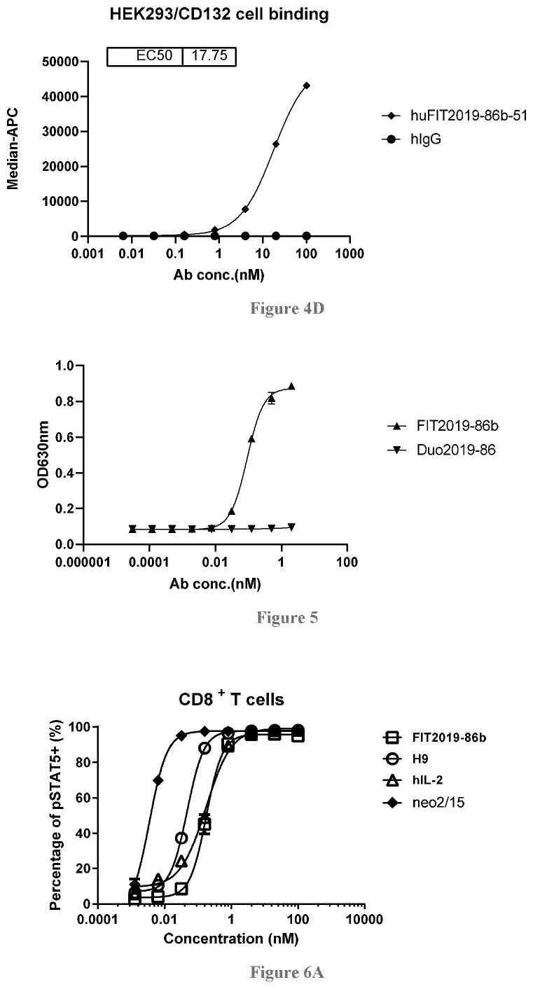

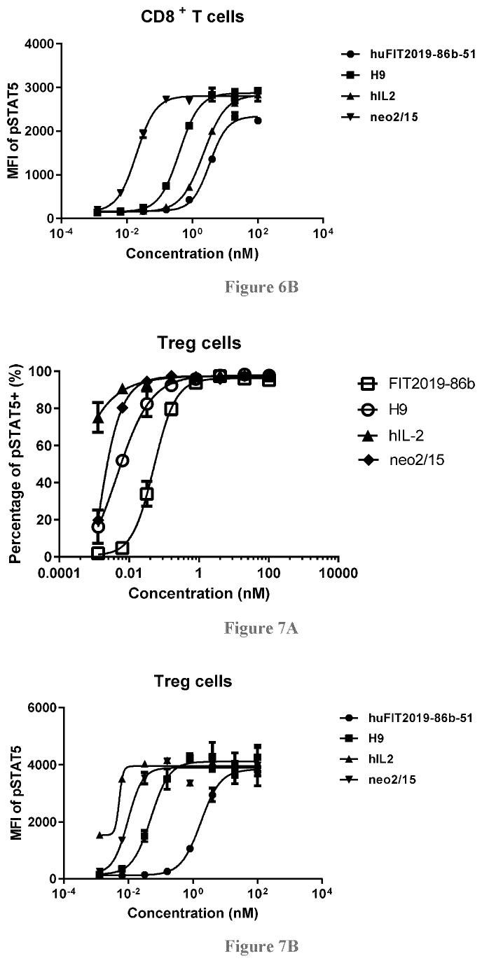

As shown in FIG. 6 and FIG. 7, treatment by IL-2, FIT2019-86b, and huFIT2019-86b-51 exhibited comparable pSTAT5 activation in human CD8+ T cells, but pSTAT5 activation in human Treg cells observed from FIT2019-86b and huFIT2019-86b-51 treatment is significantly weaker. Meanwhile, “non-CD25 binding” reference molecules (neo2/15 as described in PCT publication WO2020/005819A1; H9 as described in PCT publication WO2018/234862A1) showed greater pSTAT5 activation in human CD8+ T cells than IL-2, but had stronger pSTAT5 activation in human Treg cells than FIT2019-86b and huFIT2019-86b-51.

### 7.2 Induction of Human PBMC Proliferation

Comparison of functional activity among FIT-Igs of the present disclosure and reference molecules was further performed in a T-cell proliferation assay.

On day 1, human CD3+ T cells were resuspended with complete medium (RPMI1640 supplemented with 10% HI-FBS, 1% PenStrep and 55 μM 2-Mercaptoethanol) at a density of 2E6 cells/mL. T cells were washed twice with 1×PBS and transferred to a 6-well plate precoated with 2 μg/mL anti-CD3 antibody (Biolegend, Cat NO. 300332). Soluble anti-CD28 antibody (Biolegend, Cat No. 302934) was also added into the plate at a final concentration of 1 μg/mL. The plate was incubated at 37° C. to day 4, then T cells were washed once with 1×PBS and resuspended with complete medium to a density of 1E6 cells/mL and rested overnight.

On day 5, T cells were collected and stained with 1 μM CellTrace CSFE (Thermo, Cat No. C34554), resuspended in complete medium at a density of 2E6 cells/mL and dispensed into 96-well round bottom plates for 100 μL per well, then incubated with test articles or controls at 37° C. for 4 days.

On day 9, cells were transferred into 96-well v-bottom plates, washed once with 1X DPBS by spinning at 400g, 7° C. for 6 min, then stained with 1:1000 diluted viability dye (eBioscience, Cat No. 65-0865-14) and 1:200 diluted Fc block (BD Bioscience, Cat No. 564219) by incubation for 10 min at room temperature in the dark. After incubation, a surface staining mixture containing APC Mouse Anti-Human CD4 (BD, Cat NO. 555349), PE-Cy7 mouse anti-human CD8 (Biolegend, Cat No. 301012), BV421 Mouse anti-human CD25 (Biolegend, 356114) were added for 50 μL per well and incubated with cells at 4° C. for 20 min. After another wash with 1X DPBS, cells were fixed with 100 μL of fresh prepared Fix/Perm solution (1:3 diluted Fix/Perm concentrate, Invitrogen, Cat. No. 00-5123) for 45 min at RT in the dark. After incubation, cells were washed with Perm buffer (1:10 diluted Perm concentrate, Invitrogen, Cat No. 00-8333) and incubated with PE Mouse anti-human FoxP3 antibody (Biolegend, Cat No. 320108) for 1 h at 4° C. After incubation, the cells were centrifuged again and washed twice with Perm buffer. Subsequently, the cell pellet was resuspended in 200 μL cell staining buffer (Biolegend, Cat #420201) and analyzed by flow cytometry.

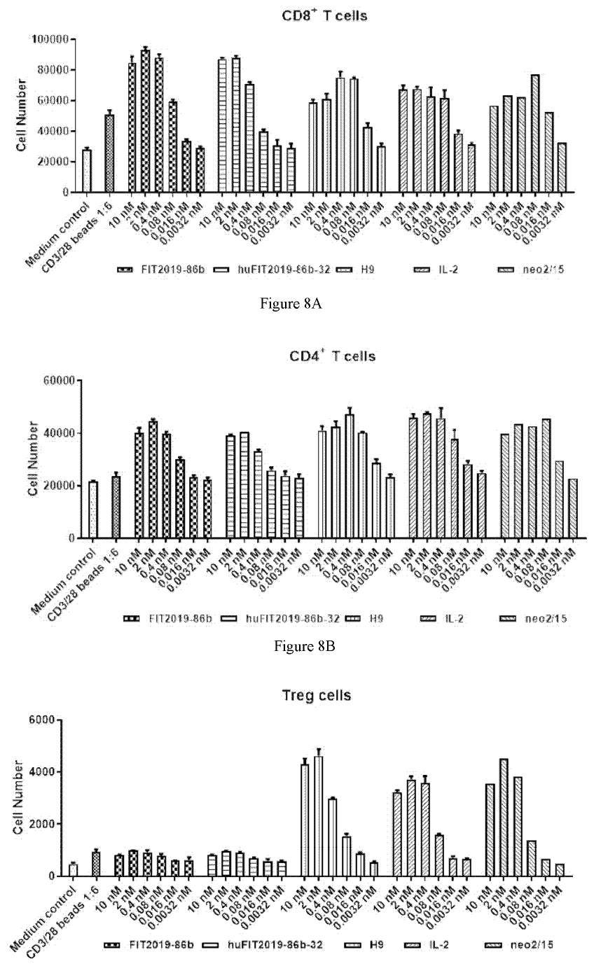

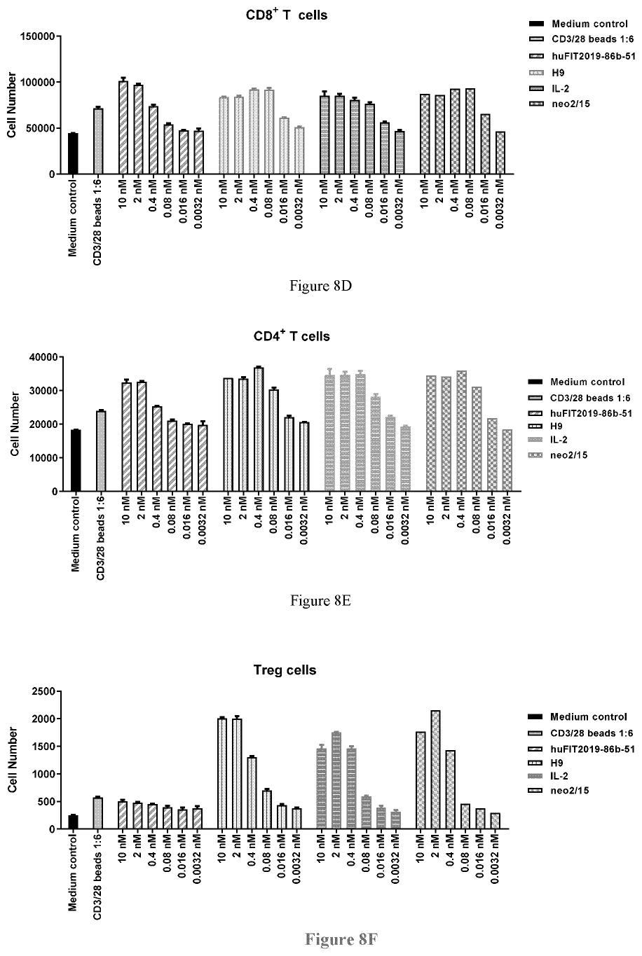

Comparing FIT-Igs (FIT2019-86b or humanized CD122/CD132 FIT-Igs generated from Example 3.2.1) with those of reference molecules (H9, neo2/15, or IL-2) by results of PBMC proliferation assay as described above, FIT-Igs of the present disclosure are comparable to the reference molecules in terms of induced proliferation of CD8+ and CD4+ T cells, but much less agonistic on proliferation of Treg cells. FIG. 8 exemplified the results from FIT2019-86b, huFIT9-86b-32 and huFIT2019-8b-51.

### Example 8. In Vivo Tumor Growth Inhibition

### 8.1 In Vivo Tumor Growth Inhibition by FIT2019-86b in Immune Compromised M-NSG Mice

In vivo tumor growth inhibition by FIT2019-86b was evaluated in a melanoma/PBMC co-implantation model in immune compromised M-NSG mice. Briefly, A375 human melanoma cells (ATCC #CRL-1619) were routinely cultured for at least two passages before transplantation. Frozen human PBMCs were thawed and recovered according to standard procedures. On transplantation day, 5×10⁶ A375 cells and 2×10⁵ PBMCs were mixed in 0.1 mL DPBS+0.1 mL Matrigel for each mouse. The resultant 0.2 mL cell suspension was injected subcutaneously (sc) into right flank of M-NSG mouse (female, 6-7 weeks old) using a 26-gauge needle. Mice with tumor volume of 80-200 mm³ were selected and randomized into two groups (day 0, DO) for treatment with 3 mg/kg FIT2019-86b or vehicle control twice a week (on D3, D7, D10, and D14). Tumor growth and body weight were measured twice a week. Tumor volume was calculated based on dimensions of the tumor using the following formula: tumor volume=(length×width²)/2. Graft versus host disease (GVHD) was assessed by measuring weight loss over time in all animals. Animals observed body weight loss of 20% or above were euthanized.

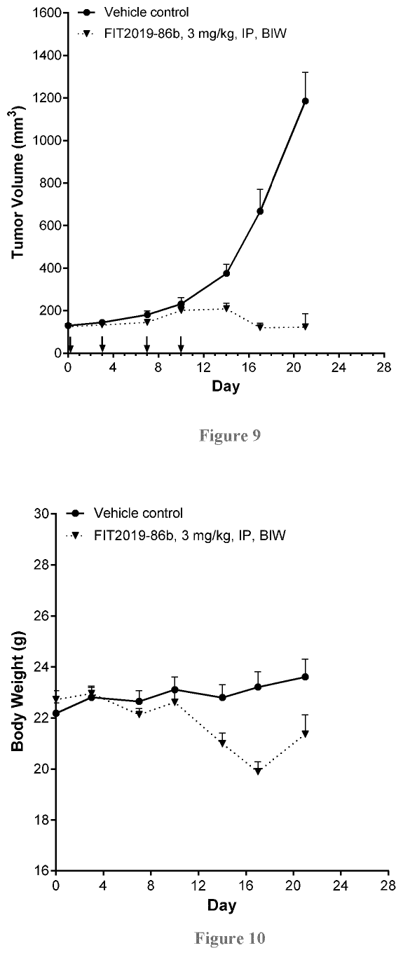

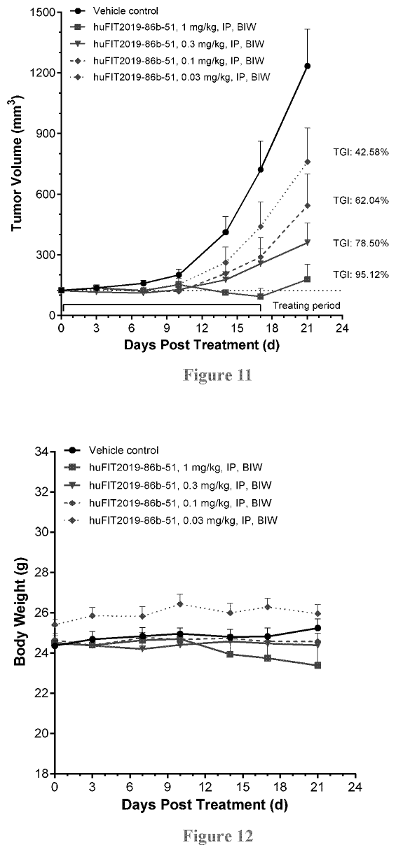

FIG. 9 demonstrates FIT2019-86b treatment resulted significant tumor growth inhibition in comparison to vehicle control. FIT2019-86b treated animals exhibited weight loss by day 14 (shown in FIG. 10), indicating an accelerated GVHD over vehicle control.

8.2 In Vivo Tumor Growth Inhibition by huFIT2019-86b-51 in Immune Compromised NCG Mice

### 8.2.1 in Melanoma/PBMC Co-Implantation Model

In vivo tumor growth inhibition by huFIT2019-86b-51 was evaluated in a melanoma/PBMC co-implantation model in immune compromised NCG mice. Briefly, A375 human melanoma cells (ATCC #CRL-1619) were routinely cultured for at least two passages before transplantation. Frozen human PBMCs were thawed and recovered according to standard procedures. On transplantation day, 5×10⁶ A375 cells and 2×10⁵ PBMCs were mixed in 0.1 mL DPBS+0.1 mL Matrigel for each mouse. The resultant 0.2 mL cell suspension was injected subcutaneously (sc) into right flank of NCG mouse (female, 6-7 weeks old) using a 26-gauge needle. Mice with tumor volume of 80-200 mm³ were selected and randomized into five groups (day 0, DO) for treatment with huFIT2019-86b-51 in four dosages (1 mg/kg, 0.3 mg/kg, 0.1 mg/kg, 0.03 mg/kg) or vehicle control twice a week, for three weeks. Tumor growth and body weight were measured twice a week. Tumor volume was calculated based on dimensions of the tumor using the following formula: tumor volume=(length×width²)/2. Graft versus host disease (GVHD) was assessed by measuring weight loss over time in all animals. Animals observed body weight loss of 20% or above were euthanized.

FIG. 11 demonstrates huFIT2019-86b-51 treatment resulted significant tumor growth inhibition in comparison to vehicle control. HuFIT2019-86b-51 (1 mg/kg) treated group exhibited slight body weight loss by day 14 (shown in FIG. 12), indicating an accelerated GVHD over vehicle control.

### 8.2.2 in NSCLC Cell/PBMC Co-Implantation Model

In vivo tumor growth inhibition by huFIT2019-86b-51 was also evaluated in a NSCLC cell/PBMC co-implantation model in immune compromised NCG mice. Briefly, H292 tumor cells (ATCC #CRL-1848) were routinely cultured for at least two passages before transplantation. Frozen human PBMCs were thawed and recovered according to standard procedures. On transplantation day, 2×10⁶ H292 cells and 4×10⁵ PBMCs were mixed in 0.1 mL DPBS for each mouse. The cell suspension was injected subcutaneously (sc) into right flank of NCG mouse (female, 6-7 weeks old) using a 26-gauge needle. Seven days post inoculation (Day 7, D7), Mice were selected and randomized into three groups for treatment with huFIT2019-86b-51 in two dosages (1 mg/kg, 0.3 mg/kg) or vehicle control twice a week (D7, D10, D14, D17 and D21). Tumor growth and body weight were measured twice a week. Tumor volume was calculated based on dimensions of the tumor using the following formula: tumor volume=(length× width²)/2. Graft versus host disease (GVHD) was assessed by measuring weight loss over time in all animals. Animals observed body weight loss of 20% or above were euthanized.

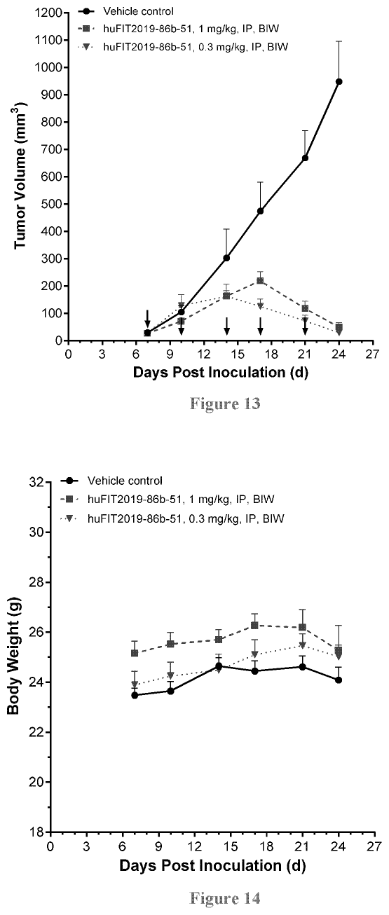

FIG. 13 demonstrates huFIT2019-86b-51 treatment resulted significant tumor growth inhibition in comparison to vehicle control. FIG. 14 shows body weight change during treatment with huFIT2019-86b-51 and vehicle control.

### Example 9. In Vivo GVHD and CD8 Expansion

The functional activity of FIT2019-86b and recombinant human IL-2 (rhIL-2) were also compared by an accelerated GVHD model and in vivo effector T cells stimulation assay. Briefly, M-NSG mice (female, 6-7 weeks old) were adoptively transferred i.p. with 5×10⁶ human PBMCs. After 18 days, the engraftment of human PBMCs was monitored by bleeding mice and measuring human CD45+ cell numbers in peripheral blood. Mice with human CD45+ percentage greater than 2% of total PBMC were selected and randomized into three groups on day 0 (DO): Group 1 (G1) mice to be treated once with vehicle control (1X PBS) on DO; Group 2 (G2) mice to be treated with 50,000 IU rhIL-2 daily for 5 consecutive days (from DO to D4); Group 3 (G3) mice to be treated with 5 mg/kg FIT2019-86b by a single injection on DO. Body weight was measured daily since DO. Fresh whole blood samples were collected from each mouse on study D1, D4 and D7. And percentages of human CD45+, CD4+ and CD8+ cells in peripheral blood were measured by flow cytometry.

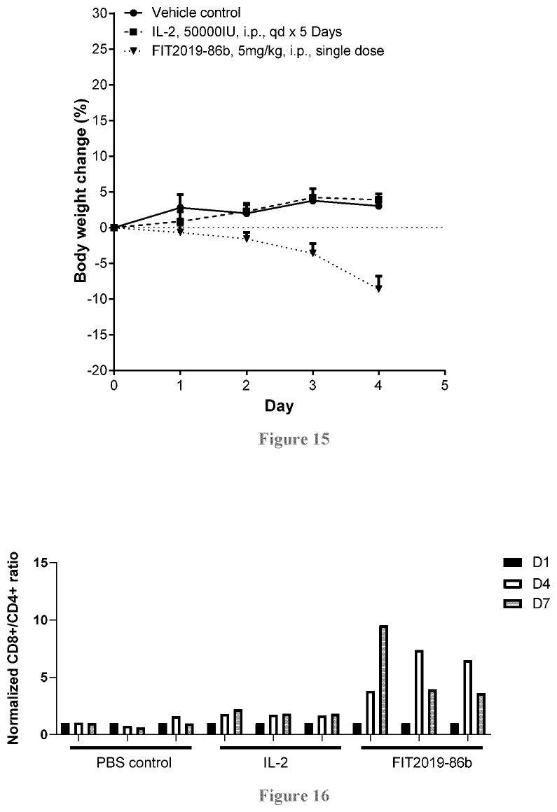

FIG. 15 shows mice treated with FIT2019-86b exhibited very quick onset of GVHD, manifested by body weight loss around D3. In contrast, vehicle control and rhIL-2 treated mice exhibited no sign of GVHD by D4. This indicates an acceleration of GVHD compared to the vehicle control and rhIL-2 groups, consistent with the enhanced activation of immune effector cells in treated mice. Particularly, response in T cells proliferation profile is shown in FIG. 16 by time course of CD8+ over CD4+ ratio normalized to the ratio of each individual mouse on D1. Relatively, FIT2019-86b treated mice showed significantly increased cytotoxic human CD8+ T cells and relatively reduced human CD4+ T cells proportion since D4, while the profiles in vehicle control group were relatively stable, and in rhIL-2 treated mice only showed mild increase of human CD8+ T cells.

### Example 10. IL-2/IL2Rβ Binding Competition Assay

The ability of huFIT2019-86b-51 and reference molecules to compete with IL2 binding to IL2Rβ was assessed by grating-coupled interferometry (GCI) with Creoptix WAVEsystem (Malvern Panalytical). 1X HBS-EP+ (Cytiva, Cat No. BR100669) with additional 350 mM NaCl was used as dilution and running buffer. Briefly, A PCP WAVEchip (Creoptix AG) was immobilized with recombinant human IL2 to a density of ˜2000 pg/mm² by amine coupling according to the manufacture's instruction. For each round of test, 500 nM IL2Rβ was firstly injected for 250s and captured by the immobilized IL2 on the chip to a density of ˜ 35 pg/mm². 500 nM test article (human IL2, H9, neo2/15 or huFIT2019-86b-51) was premixed with IL2Rβ (at 1:1 concentration ratio) and injected for 250s to assess the binding of the mixture to the IL2/IL2Rβ complex on the chip. At the end of each round, WAVEchip was regenerated by pH 1.5 Glycine-HCl buffer at 60 μL/min for 60s. Sensorgrams were recorded at 25° C., and data were analyzed with WAVEcontrol (Creoptix AG). Theoretically, if the mixture of test article/IL2Rβ showed less binding to the IL2/IL2Rβ complex than IL2Rβ alone, the test article shall be competing with IL2. If not, then the test article has a different IL2Rβ binding epitope from IL2. IL2 was used as the competing-positive control in this assay.

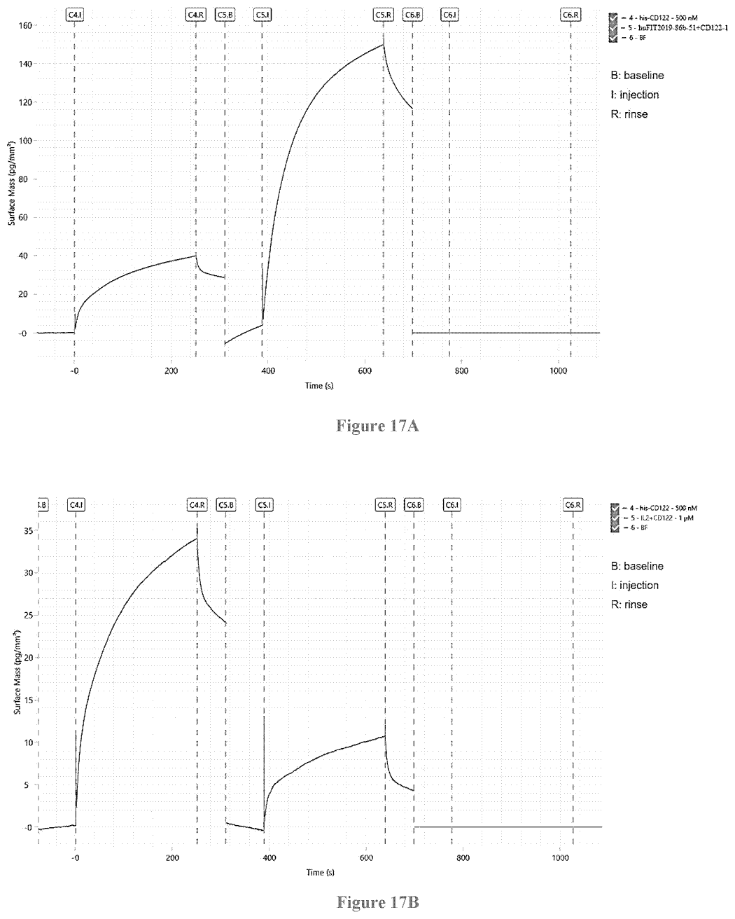

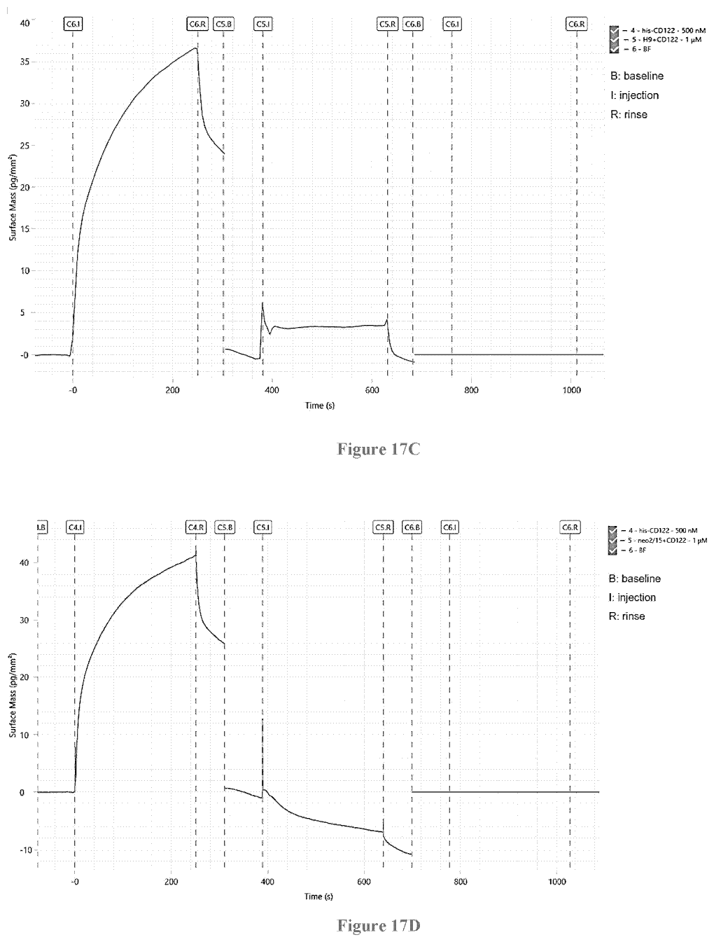

As shown in FIG. 17, unlike IL2 and its derivatives, huFIT2019-86b-51 does not compete with IL2/IL2Rβ binding.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed:

1. An isolated antibody or antigen-binding fragment thereof that specifically binds to CD122, comprising a set of six CDRs, CDR-H1, CDR-H2, CDR-H3, CDR-L1, CDR-L2, and CDR-L3, wherein:
CDR-H1 comprises the sequence of DYVIS (SEQ ID NO: 44);
CDR-H2 comprises the sequence of EIYPGDGNTYYNEMFKG (SEQ ID NO: 45), EIYPGDAQTYYNEMFKG (SEQ ID NO: 47), EIYPGDANTYYNEMFKG (SEQ ID NO: 48), or EIYPGEGNTYYNEMFKG (SEQ ID NO: 49);
CDR-H3 comprises the sequence of GSYTYDNYAMDF (SEQ ID NO: 46);
CDR-L1 comprises the sequence of RSSQNIVHSNGNTYLE (SEQ ID NO: 50), RSSQNIVHSEGQTYLE (SEQ ID NO: 53), RSSQNIVHSNANTYLE (SEQ ID NO: 54), RSSQNIVHSNGQTYLE (SEQ ID NO: 55), RSSQNIVHSNAQTYLE (SEQ ID NO: 56);
CDR-L2 comprises the sequence of KVSNRFS (SEQ ID NO: 51); and
CDR-L3 comprises the sequence of FQGSHIPWT (SEQ ID NO: 52),
optionally wherein the CDRs are defined according to Kabat numbering.

2. The isolated antibody or antigen-binding fragment of claim 1, wherein the antibody comprises a variable heavy chain domain VH and a variable light chain domain VL, wherein:
the VH domain comprises the sequence of SEQ ID NO: 3, 63, 64, or 65, or a sequence having at least 80%, 85%, 90%, 91%, 92%, 93%, 94%, 95%, 96%, 97%, 98%, 99% or more identity therewith, and/or the VL domain comprises the sequence of SEQ ID NO: 4, 66, 67, 68, or 69, or a sequence having at least 80%, 85%, 90%, 91%, 92%, 93%, 94%, 95%, 96%, 97%, 98%, 99% or more identity therewith;
or
the VH domain comprises the sequence selected from any one of SEQ ID NOs: 21-29, or a sequence having at least 80%, 85%, 90%, 91%, 92%, 93%, 94%, 95%, 96%, 97%, 98%, 99% or more identity therewith, and/or the VL domain comprises the sequence selected from any one of SEQ ID NOs: 30-43, or a sequence having at least 80%, 85%, 90%, 91%, 92%, 93%, 94%, 95%, 96%, 97%, 98%, 99% or more identity therewith.

3. The isolated antibody or antigen-binding fragment of claim 1, wherein the antibody is a chimeric or humanized antibody, optionally the antibody is a humanized antibody,
and further optionally, the VH domain of the antibody comprises amino acid residues 1E, and 1 to 9 residues selected from 28T, 30T, 38R, 48M, 67V, 69M, 72N, 73T, 91Y, according to Kabat numbering; and the VL domain comprises 1 to 4 amino acid residues selected from 7S, 36F, 37Q, and 46R, according to Kabat numbering.

4. The isolated antibody or antigen-binding fragment of claim 1, wherein the antibody comprises a combination of VH and VL sequences selected from the group consisting of:

| combination | VH sequence | VL sequence |
|---|---|---|
| 1 | SEQ ID NO: 21 | SEQ ID NO: 30 |
| 2 | SEQ ID NO: 22 | SEQ ID NO: 30 |
| 3 | SEQ ID NO: 23 | SEQ ID NO: 30 |
| 4 | SEQ ID NO: 24 | SEQ ID NO: 30 |
| 5 | SEQ ID NO: 25 | SEQ ID NO: 30 |
| 6 | SEQ ID NO: 26 | SEQ ID NO: 30 |
| 7 | SEQ ID NO: 21 | SEQ ID NO: 31 |
| 8 | SEQ ID NO: 22 | SEQ ID NO: 31 |
| 9 | SEQ ID NO: 23 | SEQ ID NO: 31 |
| 10 | SEQ ID NO: 24 | SEQ ID NO: 31 |
| 11 | SEQ ID NO: 25 | SEQ ID NO: 31 |
| 12 | SEQ ID NO: 26 | SEQ ID NO: 31 |
| 13 | SEQ ID NO: 21 | SEQ ID NO: 32 |
| 14 | SEQ ID NO: 22 | SEQ ID NO: 32 |
| 15 | SEQ ID NO: 23 | SEQ ID NO: 32 |
| 16 | SEQ ID NO: 24 | SEQ ID NO: 32 |
| 17 | SEQ ID NO: 25 | SEQ ID NO: 32 |
| 18 | SEQ ID NO: 26 | SEQ ID NO: 32 |
| 19 | SEQ ID NO: 21 | SEQ ID NO: 33 |
| 20 | SEQ ID NO: 21 | SEQ ID NO: 34 |
| 21 | SEQ ID NO: 21 | SEQ ID NO: 35 |
| 22 | SEQ ID NO: 27 | SEQ ID NO: 36 |
| 23 | SEQ ID NO: 27 | SEQ ID NO: 37 |
| 24 | SEQ ID NO: 27 | SEQ ID NO: 38 |
| 25 | SEQ ID NO: 27 | SEQ ID NO: 39 |
| 26 | SEQ ID NO: 27 | SEQ ID NO: 40 |
| 27 | SEQ ID NO: 21 | SEQ ID NO: 36 |
| 28 | SEQ ID NO: 28 | SEQ ID NO: 36 |
| 29 | SEQ ID NO: 29 | SEQ ID NO: 36 |
| 30 | SEQ ID NO: 27 | SEQ ID NO: 41 |
| 31 | SEQ ID NO: 27 | SEQ ID NO: 42 |
| 32 | SEQ ID NO: 27 | SEQ ID NO: 43 |
| 33 | SEQ ID NO: 21 | SEQ ID NO: 41 |

optionally wherein the antibody comprises a VH domain comprising the sequence of SEQ ID NO: 21 and a VL domain comprising the sequence of SEQ ID NO: 41.

5. The isolated antibody or antigen-binding fragment of any one of claims 1-4, wherein the antibody comprises an Fc region, for example, an Fc region having an amino acid sequence of SEQ ID NO: 70.

6. A fusion or a conjugate comprising the isolated antibody or antigen-binding fragment of any one of claims 1-5.

7. A method of detecting CD122 in a biological sample, comprising contacting the biological sample with the isolated antibody or antigen-binding fragment of any one of claims 1-5 or the fusion or conjugate of claim 6.

8. An isolated antibody or antigen-binding fragment thereof that specifically binds to CD132, comprising a set of six CDRs, CDR-H1, CDR-H2, CDR-H3, CDR-L1, CDR-L2, and CDR-L3, wherein:
CDR-H1 comprises the sequence of SYWMH (SEQ ID NO: 57);
CDR-H2 comprises the sequence of HIYLGGGATNYAEKFRS (SEQ ID NO: 58);
CDR-H3 comprises the sequence of SQPYYYGMDS (SEQ ID NO: 59);
CDR-L1 comprises the sequence of RASQDISNYLN (SEQ ID NO: 60);
CDR-L2 comprises the sequence of YKSRLHS (SEQ ID NO: 61); and
CDR-L3 comprises the sequence of HQGHTIPFT (SEQ ID NO: 62),
optionally wherein the CDRs are defined according to Kabat numbering.

9. The isolated antibody or antigen-binding fragment of claim 8, wherein the antibody comprises a variable heavy chain domain VH and a variable light chain domain VL, wherein:
the VH domain comprises the sequence of SEQ ID NO: 5, or a sequence having at least 80%, 85%, 90%, 91%, 92%, 93%, 94%, 95%, 96%, 97%, 98%, 99% or more identity therewith, and/or the VL domain comprises the sequence of SEQ ID NO: 6, or a sequence having at least 80%, 85%, 90%, 91%, 92%, 93%, 94%, 95%, 96%, 97%, 98%, 99% or more identity therewith;
or
the VH domain comprises the sequence selected from any one of SEQ ID NOs. 14 to 18, or a sequence having at least 80%, 85%, 90%, 91%, 92%, 93%, 94%, 95%, 96%, 97%, 98%, 99% or more identity therewith, and/or the VL domain comprises the sequence selected from any one of SEQ ID NO: 19 or 20, or a sequence having at least 80%, 85%, 90%, 91%, 92%, 93%, 94%, 95%, 96%, 97%, 98%, 99% or more identity therewith,
optionally, the VH domain of the antibody comprises amino acid residues 1E, and 1 to 8 amino acid residues selected from 37M, 38K, 48I, 66K, 67A, 69L, 71A and 78A, according to Kabat numbering; and the VL domain comprises 1 to 2 amino acid residues selected from 70E and 71Y, according to Kabat numbering,
optionally, the antibody comprises a combination of VH and VL sequences selected from the group consisting of:

| combination | VH sequence | VL sequence |
|---|---|---|
| 34 | SEQ ID NO: 14 | SEQ ID NO: 19 |
| 35 | SEQ ID NO: 15 | SEQ ID NO: 19 |
| 36 | SEQ ID NO: 16 | SEQ ID NO: 19 |
| 37 | SEQ ID NO: 17 | SEQ ID NO: 19 |
| 38 | SEQ ID NO: 18 | SEQ ID NO: 19 |
| 39 | SEQ ID NO: 14 | SEQ ID NO: 20 |
| 40 | SEQ ID NO: 15 | SEQ ID NO: 20 |
| 41 | SEQ ID NO: 16 | SEQ ID NO: 20 |
| 42 | SEQ ID NO: 17 | SEQ ID NO: 20 |
| 43 | SEQ ID NO: 18 | SEQ ID NO: 20 |

optionally wherein the antibody comprises a VH domain comprising the sequence of SEQ ID NO: 14 and a VL domain comprising the sequence of SEQ ID NO: 19.

10. The isolated antibody or antigen-binding fragment of claim 8, wherein the antibody is a chimeric or humanized antibody, optionally the antibody is a humanized antibody.

11. The isolated antibody or antigen-binding fragment of any one of claims 8-10, wherein the antibody comprises an Fc region, for example, an Fc region having an amino acid sequence of SEQ ID NO: 70.

12. A fusion or a conjugate comprising the isolated antibody or antigen-binding fragment of any one of claims 8-11.

13. A method of detecting CD132 in a biological sample, comprising contacting the biological sample with the isolated antibody or antigen-binding fragment of any one of claims 8-11 or the fusion or conjugate of claim 12.

14. A nucleic acid molecule encoding the isolated antibody or antigen-binding fragment of any one of claims 1-5 and 8-11.

15. A vector comprising the nucleic acid molecule of claim 14.

16. A host cell expressing the nucleic acid molecule encoding the isolated antibody or antigen-binding fragment of any one of claims 1-5 and 8-11.

17. A pharmaceutical composition comprising the isolated antibody or antigen-binding fragment of any one of claims 1-5 and 8-11, the fusion or conjugate of claims 6 and 12, the nucleic acid molecule of claim 14, the vector of claim 15, or the host cell of claim 16.

18. A bispecific binding protein that specifically binds CD122 and CD132, comprising a first antigen-binding site that specifically binds CD122, and a second antigen-binding site that specifically binds CD132, wherein
the first antigen-binding site comprises a set of six CDRs, CDR-H1, CDR-H2, CDR-H3, CDR-L1, CDR-L2, and CDR-L3, wherein:
CDR-H1 comprises the sequence of DYVIS (SEQ ID NO: 44);
CDR-H2 comprises the sequence of EIYPGDGNTYYNEMFKG (SEQ ID NO: 45), EIYPGDAQTYYNEMFKG (SEQ ID NO: 47), EIYPGDANTYYNEMFKG (SEQ ID NO: 48), or EIYPGEGNTYYNEMFKG (SEQ ID NO: 49);
CDR-H3 comprises the sequence of GSYTYDNYAMDF (SEQ ID NO: 46);
CDR-L1 comprises the sequence of RSSQNIVHSNGNTYLE (SEQ ID NO: 50), RSSQNIVHSEGQTYLE (SEQ ID NO: 53), RSSQNIVHSNANTYLE (SEQ ID NO: 54), RSSQNIVHSNGQTYLE (SEQ ID NO: 55), RSSQNIVHSNAQTYLE (SEQ ID NO: 56);
CDR-L2 comprises the sequence of KVSNRFS (SEQ ID NO: 51); and
CDR-L3 comprises the sequence of FQGSHIPWT (SEQ ID NO: 52),
optionally, the first antigen-binding site comprises a VH domain and a VL domain as defined in any one of claims 2-4;
and/or the second antigen-binding site comprises a set of six CDRs, CDR-H1, CDR-H2, CDR-H3, CDR-L1, CDR-L2, and CDR-L3, wherein:
CDR-H1 comprises the sequence of SYWMH (SEQ ID NO: 57);
CDR-H2 comprises the sequence of HIYLGGGATNYAEKFRS (SEQ ID NO: 58);
CDR-H3 comprises the sequence of SQPYYYGMDS (SEQ ID NO: 59);
CDR-L1 comprises the sequence of RASQDISNYLN (SEQ ID NO: 60);
CDR-L2 comprises the sequence of YKSRLHS (SEQ ID NO: 61); and
CDR-L3 comprises the sequence of HQGHTIPFT (SEQ ID NO: 62),
optionally, the second antigen-binding site comprises a VH domain and a VL domain as defined in any one of claims 8-10;
wherein the CDRs are defined according to Kabat numbering.

19. The bispecific binding protein of claim 18, comprising a first polypeptide chain, a second polypeptide chain and a third polypeptide chain,
wherein the first polypeptide chain comprises, from amino terminus to carboxyl terminus, VL_{A}-CL-VH_{B}-CH1-hinge-CH2-CH3; the second polypeptide chain comprises, from amino to carboxyl terminus, VH_{A}-CH1; the third polypeptide chain comprises, from amino to carboxyl terminus, VL_{B}-CL;
wherein the VL_{A}-CL pairs with VH_{A}-CH1 to form a first Fab that specifically binds a first antigen A, and VL_{B}-CL pairs with VH_{B}-CH1 to form a second Fab that specifically binds a second antigen B, and
wherein the first antigen A and the second antigen B are CD122 and CD132 respectively, optionally the first antigen A is CD122 and the second antigen B is CD132;
wherein said three polypeptide chains as a half molecule associates with another said three polypeptide chains as the other half molecule to form a FIT-Ig protein.

20. The bispecific binding protein of claim 18 in a duobody format based on Fab arm exchange of the antibody of any of claims 1-4 and the antibody of any of claims 8-10, optionally having (i) a heavy chain comprising an amino acid sequence of SEQ ID NO: 10, or a sequence having at least 80%, 85%, 90%, 91%, 92%, 93%, 94%, 95%, 96%, 97%, 98%, 99% or more identity therewith, and a pairing light chain comprising an amino acid sequence of SEQ ID NO: 11, or a sequence having at least 80%, 85%, 90%, 91%, 92%, 93%, 94%, 95%, 96%, 97%, 98%, 99% or more identity therewith, and (ii) a heavy chain comprising an amino acid sequence of SEQ ID NO: 12, or a sequence having at least 80%, 85%, 90%, 91%, 92%, 93%, 94%, 95%, 96%, 97%, 98%, 99% or more identity therewith, and a pairing light chain comprising an amino acid sequence of SEQ ID NO: 13, or a sequence having at least 80%, 85%, 90%, 91%, 92%, 93%, 94%, 95%, 96%, 97%, 98%, 99% or more identity therewith.

21. The bispecific binding protein of claim 19, wherein:
the first polypeptide chain comprises an amino acid sequence of SEQ ID NO: 7, or a sequence having at least 80%, 85%, 90%, 91%, 92%, 93%, 94%, 95%, 96%, 97%, 98%, 99% or more identity therewith,
the second polypeptide chain comprises an amino acid sequence of SEQ ID NO: 8, or a sequence having at least 80%, 85%, 90%, 91%, 92%, 93%, 94%, 95%, 96%, 97%, 98%, 99% or more identity therewith, and
the third polypeptide chain comprises an amino acid sequence of SEQ ID NO: 9, or a sequence having at least 80%, 85%, 90%, 91%, 92%, 93%, 94%, 95%, 96%, 97%, 98%, 99% or more identity therewith.

22. The bispecific binding protein of any one of claims 19-21, wherein the bispecific binding protein has one or more of the following characteristics:
(i) binding to CD122-expressing cells, wherein said cell binding potency is reflected by an EC50 of about 5 nM or lower, 4 nM or lower, 3 nM or lower, 2 nM or lower, or 1 nM or lower, as measured by flow cytometry in a cell-based assay;
(ii) binding to CD132-expressing cells, wherein said cell binding potency is reflected by an EC50 of about 80 nM or lower, 60 nM or lower, 40 nM or lower, 20 nM or lower, or 10 nM or lower, as measured by flow cytometry in a cell-based assay;
(iii) stimulating signaling upon binding to a complex comprising CD122 and CD132;
(iv) binding to human CD122 and human CD132 with a K_{D} of less than about 30 nM, 25 nM, 20 nM, 15 nM, 10 nM, or 5 nM; and is cross-reactive with cynomolgus CD122 and cynomolgus CD132 with a K_{D} of less than about 100 nM, 80 nM, 60 nM, 40 nM, 20 nM, or 10 nM, as measured by a WAVEsystem or Biacore assay;
(v) stimulating proliferation of cells expressing CD122 and CD132;
(vi) preferentially stimulating proliferation of CD8+ and/or CD4+ T cells over regulatory T cells;
(vii) improving anti-tumor immunity of effector T cells and/or NK cells in vivo and/or in vitro, e.g., reducing tumor burden/growth/cell expansion, optionally wherein said anti-tumor immunity comprises anti-tumor cytotoxicity.

23. A nucleic acid molecule encoding the bispecific binding protein of any one of claims 19-22.

24. A vector comprising the nucleic acid molecule of claim 23.

25. A host cell comprising the nucleic acid molecule of claim 23, or the vector of claim 24.

26. A method of preparing the isolated antibody or antigen-binding fragment of any one of claims 1-6 and 8-11, or the bispecific binding protein of any one of claims 19-22, comprising:
culturing the host cell of claim 15 or claim 25 under conditions that allow the production of the antibody, antigen-binding fragment, or bispecific binding protein; and
recovering the antibody, antigen-binding fragment, or bispecific binding protein from the culture.

27. A pharmaceutical composition comprising the bispecific binding protein of any one of claims 19-22, the nucleic acid of claim 23, the vector of claim 24, or the host cell of claim 25.

28. A method of treating or preventing a disease wherein the functions of effector T cells and/or NK cells are impaired and the immune responses are down regulated, comprising administering to a subject in need thereof a therapeutically effective amount of the pharmaceutical composition of claim 17 or claim 27.

29. The method of claim 28, wherein the subject is a human.

30. The method of claim 28, wherein the disease is a T cell dysfunctional disorder or a cancer, for example, melanoma, metastatic melanoma, renal cell carcinoma, ovarian cancer, lung cancer, small cell lung cancer, non-small cell lung cancer, brain cancer, head and neck cancer, breast cancer, colon cancer, colorectal cancer, cervical carcinoma, hepatocellular carcinoma, prostate cancer, or bladder cancer.
